SEQUENCE LISTING (36 sequences)

SEQ ID NO: 1 - DNA, 20 bp, synthetic construct
  source 1..20: mol_type = other DNA
cgcatacagtcttrcaggct

SEQ ID NO: 2 - DNA, 24 bp, synthetic construct
  source 1..24: mol_type = other DNA
gtgtgatgttgawatgacatggtc

SEQ ID NO: 3 - DNA, 28 bp, synthetic construct
  source 1..28: mol_type = other DNA
  misc_feature 1: note = Fluorescein (FAM)
  misc_feature 28: note = Iowa black FQ Dark Quencher
ttaagatgtggtgcttgcatacgtagac

SEQ ID NO: 4 - DNA, 20 bp, synthetic construct
  source 1..20: mol_type = other DNA
gatggccccaatcacaggaa

SEQ ID NO: 5 - DNA, 20 bp, synthetic construct
  source 1..20: mol_type = other DNA
cagactcaatttagtgagcc

SEQ ID NO: 6 - DNA, 18 bp, synthetic construct
  source 1..18: mol_type = other DNA
ctgctggtccatgctgtt

SEQ ID NO: 7 - DNA, 21 bp, synthetic construct
  source 1..21: mol_type = other DNA
gaggaatgcagactcagtgga

SEQ ID NO: 8 - DNA, 21 bp, synthetic construct
  source 1..21: mol_type = other DNA
tagcttccactgctacctccg

SEQ ID NO: 9 - DNA, 20 bp, synthetic construct
  source 1..20: mol_type = other DNA
tcgtatgccacgctcatctg

SEQ ID NO: 10 - DNA, 20 bp, synthetic construct
  source 1..20: mol_type = other DNA
cggattgaattctgccaggt

SEQ ID NO: 11 - DNA, 20 bp, synthetic construct
  source 1..20: mol_type = other DNA
gctgcaaacccaatttagga

SEQ ID NO: 12 - DNA, 22 bp, synthetic construct
  source 1..22: mol_type = other DNA
tgctcatcaagttcagaaagga

SEQ ID NO: 13 - DNA, 20 bp, synthetic construct
  source 1..20: mol_type = other DNA
cctacaaagctgaggcctgt

SEQ ID NO: 14 - DNA, 20 bp, synthetic construct
  source 1..20: mol_type = other DNA
gaccctcctctcaatggtgc

SEQ ID NO: 15 - DNA, 24 bp, synthetic construct
  source 1..24: mol_type = other DNA
agcatcattaagctcatggaaaaa

SEQ ID NO: 16 - DNA, 20 bp, synthetic construct
  source 1..20: mol_type = other DNA
gtggactcaaagcatggcag

SEQ ID NO: 17 - DNA, 23 bp, synthetic construct
  source 1..23: mol_type = other DNA
cattggagcaagtgttggatctt

SEQ ID NO: 18 - DNA, 22 bp, synthetic construct
  source 1..22: mol_type = other DNA
gagctaatgcatgccattctca

SEQ ID NO: 19 - RNA, 21 nt, synthetic construct
  source 1..21: mol_type = other RNA
ctggaactggacttcagggtt

SEQ ID NO: 20 - DNA, 20 bp, synthetic construct
  source 1..20: mol_type = other DNA
cactccctcggctgtagttg

SEQ ID NO: 21 - DNA, 20 bp, synthetic construct
  source 1..20: mol_type = other DNA
ggctctttggtgtaacggga

SEQ ID NO: 22 - DNA, 23 bp, synthetic construct
  source 1..23: mol_type = other DNA
tgtctccaacacaaaatctctga

SEQ ID NO: 23 - DNA, 22 bp, synthetic construct
  source 1..22: mol_type = other DNA
ctacgagcccaagaaagatgcc

SEQ ID NO: 24 - DNA, 21 bp, synthetic construct
  source 1..21: mol_type = other DNA
gcccacttacttgcattgctg

SEQ ID NO: 25 - DNA, 20 bp, synthetic construct
  source 1..20: mol_type = other DNA
gaggtgcaatgtggggagaa

SEQ ID NO: 26 - DNA, 20 bp, synthetic construct
  source 1..20: mol_type = other DNA
ttctcaaggtctgcgtccac

SEQ ID NO: 27 - DNA, 20 bp, synthetic construct
  source 1..20: mol_type = other DNA
cactaccaggaggccgagaa

SEQ ID NO: 28 - DNA, 20 bp, synthetic construct
  source 1..20: mol_type = other DNA
ttggggtaggggtcctgtag

SEQ ID NO: 29 - DNA, 20 bp, synthetic construct
  source 1..20: mol_type = other DNA
gtgagttcgattagccgcgt

SEQ ID NO: 30 - DNA, 19 bp, synthetic construct
  source 1..19: mol_type = other DNA
gtccagccggttccctttg

SEQ ID NO: 31 - DNA, 21 bp, synthetic construct
  source 1..21: mol_type = other DNA
ggagcgagatccctccaaaat

SEQ ID NO: 32 - DNA, 23 bp, synthetic construct
  source 1..23: mol_type = other DNA
ggctgttgtcatacttctcatgg

SEQ ID NO: 33 - DNA, 22 bp, synthetic construct
  source 1..22: mol_type = other DNA
tggaccttgaaggaaaacaggg

SEQ ID NO: 34 - DNA, 22 bp, synthetic construct
  source 1..22: mol_type = other DNA
tggttctaaagccgaaaaaccc

SEQ ID NO: 35 - DNA, 22 bp, synthetic construct
  source 1..22: mol_type = other DNA
  misc_feature 22: note = Hexachloro Fluorescein
ctattaatttagtgcgtgatct

SEQ ID NO: 36 - DNA, 22 bp, synthetic construct
  source 1..22: mol_type = other DNA
  misc_feature 22: note = Fluoroscein (FAM)
ttatagtgcgtgagccagaaga

(19) United States
(12) Patent Application Publication
(10) Pub. No.: US 2026/0209713 A1
(43) Pub. Date: Jul. 23, 2026

(54) HUMAN NASAL ORGANOIDS AND METHODS OF MAKING AND METHODS OF USE THEREOF

(71) Applicants: University of Hong Kong Versitech Limited, Hong Kong (CN); Centre for Virology, Vaccinology and Therapeutics Limited, Hong Kong (CN)

(72) Inventors: Jie ZHOU, Hong Kong (CN); Man Chun CHIU, Hong Kong (CN); Cun LI, Hong Kong (CN); Kwok Yung YUEN, Hong Kong (CN)

(21) Appl. No.: 18/881,591

(22) PCT Filed: Jul. 5, 2023
(86) PCT No.: PCT/CN2023/105862

§ 371 Date: Jan. 6, 2025

Related U.S. Application Data
(60) Provisional application No. 63/358,795, filed on Jul. 6, 2022.

(51) Int. Cl. C12N 5/071

(52) U.S. Cl. CPC C12N 5/0688; C12N 2501/117; C12N 2501/119; C12N 2501/155; C12N 2501/415; C12N 2503/04; C12N 2513/00

(57) ABSTRACT

Provided is a robust nasal organoid culture system and fully characterized 2D and 3D in-vitro generated organoids, and methods of using the organoids. The nasal organoids contain a population of differentiated nasal epithelial cells having cellular heterogeneity and molecular markers similar to mature nasal epithelial tissue. Improved methods for generating two-dimensional and three-dimensional nasal organoids are also provided. The methods generally require a two-phase culture system, i.e., expansion and differentiation culture. The provided nasal organoids are suitable for recapitulating nasal tissue complexity for modeling respiratory diseases, studying barrier function and drug delivery, high-throughput drug screening, and assessing the effects of pathogens and environmental toxins on the nasal respiratory epithelium.

### CROSS REFERENCE TO RELATED APPLICATIONS

This application claims benefit of and priority to U.S. Provisional Application No. 63/358,795, filed Jul. 6, 2022, which is specifically incorporated by reference herein in its entirety.

### REFERENCE TO THE SEQUENCE LISTING

The Sequence Listing submitted as a text file named “UHK_01234_PCT_ST26.xml” created on Jun. 27, 2023, and having a size of 34,301 bytes is hereby incorporated by reference pursuant to 37 C.F.R. § 1.834 (c) (1).

### FIELD OF THE INVENTION

The disclosed invention is generally in the field of organoids and specifically in the area of human nasal organoids.

### BACKGROUND OF THE INVENTION

The COVID-19 pandemic has caused unprecedented health and socioeconomic crisis since late 2019 (1, 2). Although COVID-19 patients have a risk of developing life-threatening pneumonia, most patients have mild to moderate symptoms, including cough, sore throat, and occasionally anosmia, due to the involvement of the upper respiratory tract. Clinically, pre-symptomatic and symptomatic COVID-19 patients have a high viral load in nasal swabs, indicating robust viral growth in the nasal epithelium and the high transmissibility of SARS-CoV-2 (3). The human upper respiratory tract, specifically the nasopharyngeal epithelium, is the entry portal and primary site for SARS-CoV-2 replication and transmission (4, 5). Particularly, nasal ciliated cells are the primary target for SARS-CoV-2 replication, especially in the early stage of infection (5).

The human airways, from the nasal cavity to terminal bronchioles, are covered with the airway epithelium, also known as the pseudostratified ciliated epithelium which consists of four major cell types: ciliated, goblet, club, and basal cells. Single-cell RNA sequencing technology has enabled an in-depth illumination of the global molecular profile of in vivo human cells, and remarkably advanced the prior understanding of human cells based on traditional characterization approaches. Single-cell sequencing studies of human airways demonstrated that the airway epithelium lining the nasal cavity differs from that covering the tracheobronchial region (6, 7). Goblet cells were more abundant in the nasal epithelium than in the tracheobronchial epithelium. In addition, the same cell types in the nose showed a differential gene expression profile from those in tracheobronchial airways, indicating region-specific sub-clusters of airway epithelial cell types in nasal and tracheobronchial compartments. Overall, nasal epithelial cells and the tracheobronchial counterparts have subtle but varied molecular profiles, which may lead to phenotypic variations accordingly.

Since late 2020, SARS-CoV-2 has undergone a series of mutations, including “variants of concern” (VOC) that affect virus transmissibility and antigenicity. Epidemiological data demonstrated that the Delta variant (B1.617.2), first identified in December 2020, was 55% more transmissible than the earlier Alpha variant (B1.1.7) (8). Subsequently, the Omicron variant (B1.1.529) emerged in November 2021; it rapidly swept across the world and has become the predominant variant (9). As the pandemic evolves, new variants would emerge inevitably. However, commonly used cell lines for studying human respiratory viruses such as A549 or Calu3 are implausible to simulate the multicellular complexity and functional diversity of human respiratory epithelium, let alone model respiratory infections. Air-liquid interface cultures of primary human airway and nasal epithelial cells develop mucociliary differentiation into pseudostratified airway epithelium. These epithelial cells have been used to study SARS-CoV-2 respiratory infection (2, 10-16). However, primary epithelial cells are unable to be long-term expanded due to limited in vitro proliferation capacity, the so-called “Hayflick limit” (17), which substantially restricts their application for routine research purposes. Thus, there is a critical need for improved cell models of the human nasal epithelium for research purposes. There is also a pressing need for improved cell models for treatments of respiratory issues.

Therefore, it is an object of the present invention to provide improved compositions of two-dimensional and three-dimensional nasal organoids.

It is another object of the invention to provide improved methods for generating two-dimensional and three-dimensional nasal organoids.

It is also an object of the present invention to provide methods of using the nasal organoids for the study of nasal biology, biomarker identification, and evaluation of new and existing treatments.

### SUMMARY OF THE INVENTION

Disclosed are human nasal organoids derived from human nasal cells. The non-limiting examples illustrate that the disclosed human nasal organoids could simulate the nasal epithelium better than the airway organoids derived from lung tissues. The disclosed human nasal organoids are derived from nasal epithelial cells extracted via a non-invasive procedure to generate nasal organoids.

The inventors have established the first adult stem cell-derived human lung organoids directly from primary lung tissues (18-22). These lung organoids were stably expanded over one year, without any feeder cells and tedious cell purification procedures. We then induced proximal and distal differentiation in the long-term expandable lung organoids and generated mature airway and alveolar organoids that morphologically and functionally phenocopy the native airway and alveolar epithelium, respectively (18, 22).

These physiologically-active respiratory organoids have become a robust and popular research tool for studying SARS-CoV-2 and other respiratory viruses (22-26). As aforementioned, the airway epithelium in the nasal mucosa differs from that covering the tracheobronchial region (6, 7). Prior studies in human intestinal organoids, the first adult stem cell-derived organoids (27, 28), indicated that organoids generated from different intestinal segments retain the structural and functional characteristics from which they are derived (29).

Disclosed is a robust nasal organoid culture system and fully characterized the organoids, and methods of using the organoids. The nasal organoids were stably passaged for more than 6 months, providing a renewable source of nasal epithelial cells. Although not intended to be limiting, the following are exemplary features of the disclosed nasal organoids: (1) the nasal organoids were and can be derived from a nasal epithelial cells obtained via a nasal swab; (2) the organoids can have long-term expandability (i.e., they can be passaged continuously up to six months or more); (3) the organoids can produce multiple cell types including ciliated cells, basal cells, goblet cells, and club cells; (4) the organoids were and can be extensively characterized via, for example, light microscopy for brightfield images and video of beating cilia, RT-qPCR for cell-type specific markers and compartment-specific markers, IF staining for cell-type identification and formation of tight junction, scanning electron microscopy for ultrastructural images of cilia, TEER for barrier integrity, flow cytometry for cell-type composition and expression of host dependency factor; (5) the organoid types can include 3D dNSO, 2D dNSO (at pH 7.4), and 2D optimized dNsO (pH 6.6/7.4); and (6) replication kinetics of the organoids were and can be extensively characterized via, for example, RT-qPCR and TCID₅₀-~3-4 log 10 unit for ancestral viruses and ~5-6 log 10 unit for Delta/Omicron variants.

The disclosed 2D and 3D nasal organoid monolayers were and can be optimized by mimicking the native microenvironment of the nasal epithelium. It was demonstrated that the optimized nasal organoid monolayers adequately recapitulate SARS-CoV-2 high infectivity, particularly the highly transmissible Delta and Omicron variants, in the upper respiratory tract. The disclosed nasal organoid monolayers are superior to most, if not all, existing respiratory organoid models. A robust organoid system was generated enabling stable expansion and reconstruction of the human nasal epithelium in culture plates. The nasal organoid culture system provides an unlimited source of physiologically active nasal epithelial cells for studying respiratory pathogens, circumventing the restriction of cultured primary nasal epithelial cells. More importantly, the facile procedure of procuring nasal cells and a zero-failure establishment efficiency allow researchers to establish personalized nasal organoids readily, which will pave a new avenue for many exciting organoid-based investigations for combating the COVID-19 pandemic.

### BRIEF DESCRIPTION OF THE DRAWINGS

FIG. 1A is schematic diagram outlining the derivation, expansion, and differentiation of human nasal organoids. The upper panel shows the derivation of nasal organoids (NsO) from nasal cells. 3D undifferentiated nasal organoids undergo expansion in the expansion (Exp) medium. Photomicrographs show growing organoids on day 0 (DO), day 3, day 7, and day 10 (magnification 40×). The bottom panel shows nasal organoids undergoing differentiation protocols to generate 3D differentiated nasal organoids (3D dNsO) or differentiated nasal organoid monolayer (dNsO-mono). Photomicrographs present differentiating 3D organoids on day 0, day 2, day 4, day 6, and day 10 in basal medium and PD medium sequentially (magnification 40×). Photomicrographs show differentiating organoid monolayers on day 2, day 4, and day 10 in expansion medium and PD medium sequentially (magnification 100×). FIG. 1B is a bar graph showing the normalized expression of cell-specific marker genes and SARS-CoV-2 receptor ACE2 genes in the parental 3D nasal organoids (NsO) and 3D differentiated nasal organoids (3D dNsO). Data represent the mean and s.d. of a representative experiment, n=4. Two-tailed unpaired Student's t-test. The experiment was independently performed in four organoid lines. FIGS. 1C-1H are bar graphs showing the expression levels of trachea/bronchi-enriched genes and nose-enriched genes of three lines of 3D differentiated human airway organoids (AwO) and other three lines of 3D differentiated nasal organoids (dNsO). Data represent the mean and s.d., n=2 in each of the organoid lines. Two-tailed unpaired Student's t-test. FIG. 1I is a bar graph showing the expression level of cell-specific marker genes and SARS-CoV-2 receptor ACE2 in parental 3D nasal organoids (NsO) and the differentiated nasal organoid monolayers (dNsO-mono). FIG. 1J is a line graph showing the trans-epithelial electrical resistance (TEER) of the dNsO-mono on the Transwell insert as measured every other day over 10 days. Blank insert without cell was measured as the baseline reading. Data represent the mean and s.d. of a representative experiment, n=4. Two-tailed unpaired Student's t-test. The experiment was independently performed in two organoid lines.

FIG. 2A is a schematic outlining the generation of optimized differentiated nasal organoid monolayers (dNsO-mono). FIG. 2B is a line graph showing the trans-epithelial electrical resistance (TEER) of the dNsO-mono differentiated at pH7.4/7.4 or pH6.6/7.4 PD medium were measured every other day over 10 days. A blank insert without any cell was measured as the baseline. Data represent the mean and s.d. of a representative experiment, n=4. Two-tailed unpaired Student's t-test. The experiment was independently performed in two organoid lines. FIGS. 2C-2V are graphs showing results from optimization experiments of differentiated nasal organoid monolayers. Parental 3D NsO, 3D dNsO, and dNsO-mono differentiated at pH 7.4/7.4 or pH 6.6/7.4 were applied to flow cytometry to examine the abundance of CK5+ basal cells, ACCTUB+ ciliated cells, MUC5AC+ goblet cells, and CC10+ club cells. FIGS. 2S-2V are representative histograms of the data in FIGS. 2C-2R. Data on the right represent the mean and s.d. of a representative experiment, n=4. Ordinary one-way ANOVA with Tukey's multiple comparison test. The experiment was independently performed in two organoid lines.

FIGS. 3A-3J are graphs showing the expression of ACE2 and TMPRSS2. The organoids were applied to flow cytometry to examine the expression of ACE2 and TMPRSS2. Representative histograms are shown in FIGS. 31 and 3J. Red, cells stained with specific antibodies; blue, cells stained with isotype controls. Data on the right represent the mean and s.d. of a representative experiment, n=4. Ordinary one-way ANOVA with Tukey's multiple comparison test. The experiment was independently performed in two organoid lines.

FIGS. 4A-4P are graphs showing results from SARS-CoV-2 infection and replication fitness in differentiated nasal organoids. FIGS. 4A-4D are bar graphs showing that at the indicated hours post-inoculation, culture media were harvested from four lines of 3D dNsO (top, 1 MOI) or dNsO-mono (bottom, 0.1 MOI) infected with SARS-CoV-2 and applied to viral load detection of RdRp gene by RT-qPCR and viral titration by TCID50 assay. Data represent mean and s.d., n=2 in each of the organoid lines. The dashed line indicates the detection limit. FIG. 4E and FIG. 4F are bar graphs showing that at the indicated hours post-inoculation with wildtype (WT), Delta, or Omicron variant (0.1 MOI), culture media were harvested from the top chambers of the dNsO-mono and applied to viral load detection and viral titration. Data show mean and s.d. of a representative experiment, n=3. Multiple unpaired t-test with multiple comparisons using Holm-Sidak method. The experiment was independently performed in two organoid lines. In FIG. 4G, at 24 hours post-inoculation (1 MOI), SARS-CoV-2 WT, Delta, or Omicron infected dNsO-mono were dissociated and applied to flow cytometry to detect dsRNA+ cells. Data represent the mean and s.d. of a representative experiment, n=4. Ordinary one-way ANOVA with Tukey's multiple comparison test. The experiment was independently performed in two organoid lines. In FIG. 4H and FIG. 4I, at the indicated hours post-inoculation with 1:1 mixture of Omicron and Delta (FIG. 4H), or mixture of Omicron and WT viruses (FIG. 41) (total 0.1 MOI), culture media were harvested from the top chambers of the dNsO-mono and applied to viral load detection using variant specific primers and probes. Data represent the mean and s.d. of a representative experiment, n=3. In FIGS. 4J and 4K, at the indicated hours post-inoculation with wildtype (WT), Delta, or Omicron variant (0.1 MOI), culture media were harvested from the apical chambers of the airway organoid monolayer (AwO-mono) and applied to viral load detection and viral titration. Data show the mean and s.d. of a representative experiment, n=3. Multiple unpaired t-test with multiple comparisons using Holm-Sidak method. FIG. 4L is a bar graph showing at the indicated hours post-inoculation with 1:1 mixture of Omicron and WT (total 0.1 MOI), culture media were harvested from the apical chambers of the AwO-mono and applied to viral load detection using variant-specific primers and probes. Data represent the mean and s.d. of a representative experiment, n=3. FIGS. 4M and 4N are bar graphs showing that at the indicated hours post-inoculation with wildtype (WT), Delta, or Omicron variant (0.01 MOI), culture media were harvested from the Vero E6/TMPRSS2 cells and applied to viral load detection and viral titration. Data show the mean and s.d. of a representative experiment, n=3. Multiple unpaired t-test with multiple comparisons using Holm-Sidak method. The experiment was independently performed in two organoid lines. FIGS. 4O and 4P are bar graphs showing that at the indicated hours post-inoculation with a 1:1 mixture of Omicron and Delta (FIG. 40), or a mixture of Omicron and WT viruses (FIG. 4P) (total 0.01 MOI), culture media were harvested from the Vero E6/TMPRSS2 cells and applied to viral load detection using variant-specific primers and probes. Data represent the mean and s.d. of a representative experiment, n=3. The experiment was independently performed in two organoid lines.

FIGS. 5A-5C are bar graphs showing that SARS-CoV-2 targets ciliated cells. In FIGS. 5A and 5B, at 24 hours post-inoculation (1 MOI), mock-, WT-, Delta-, or Omicron-infected dNsO-mono were dissociated and applied to flow cytometry to detect ACCTUB+ ciliated cells. Data represent the mean and s.d. of a representative experiment, n=4. Ordinary one-way ANOVA with Tukey's multiple comparison test. The experiment was independently performed in two organoid lines. In FIG. 5C, at 24 hours post-inoculation (1 MOI), mock-, WT-, or Delta-infected airway organoid monolayer (AwO-mono) were dissociated and applied to flow cytometry to detect ACCTUB+ ciliated cells. Data represent the mean and s.d. of a representative experiment, n=3. Ordinary one-way ANOVA with Tukey's multiple comparison test. The experiment was independently performed in two organoid lines.

FIGS. 6A-6H are bar graphs showing SARS-CoV-2 damages cellular junctions. FIGS. 6A-6C are bar graphs showing image quantification of the immunofluorescence labeled mock-, WT-, or Delta-infected dNsO-mono. Geometric mean intensity relative to OCLN (FIG. 6A), ZO-1 (FIG. 6B), and F-actin (FIG. 6C) in mock-infected organoids is shown. Data represent the mean and s.d. of a representative experiment, n=3. Ordinary one-way ANOVA with Tukey's multiple comparison test. FIG. 6D is a bar graph showing that at 24 hours post-inoculation (1 MOI), mock-, WT-, Delta-, or Omicron-infected dNsO-mono were dissociated and applied to flow cytometry to detect OCLN+ cells. Data represent the mean and s.d. of a representative experiment, n=4. Ordinary one-way ANOVA with Tukey's multiple comparison test. The experiment was independently performed in two organoid lines. FIGS. 6E-6G show image quantification of the immunofluorescence labeled mock-, WT-, or Delta-infected AwO-mono. Geometric mean intensity relative to mock of OCLN (FIG. 6E), ZO-1 (FIG. 6F), and F-actin (FIG. 6G) were shown. Data represent the mean and s.d. of a representative experiment, n=3. Ordinary one-way ANOVA with Tukey's multiple comparison test. FIG. 6H show that at 24 hours post-inoculation (1 MOI), mock-, WT-, or Delta-infected AwO-mono were dissociated and applied to flow cytometry to detect OCLN+ cells. Data represent the mean and s.d. of a representative experiment, n=3. Ordinary one-way ANOVA with Tukey's multiple comparison test. The experiment was independently performed in two organoid lines.

### DETAILED DESCRIPTION OF THE INVENTION

The disclosed method and compositions can be understood more readily by reference to the following detailed description of particular embodiments and the Example included therein and to the Figures and their previous and following description.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### I. Definitions

“Organoid” as used herein refers to an artificial, in vitro construct derived from adult stem cells created to mimic or resemble the functionality and/or histological structure of an organ or portion thereof.

“Media” or “culture media” as used herein refers to an aqueous based solution that is provided for the growth, viability, or storage of cells used in carrying out the present invention. A media or culture media may be natural or artificial. A media or culture media may include a basal medium and may be supplemented with nutrients (e.g., salts, amino acids, vitamins, trace elements, antioxidants) to promote the desired cellular activity, such as cell viability, growth, proliferation, and/or differentiation of the cells cultured in the media. A “basal medium,” as used herein, refers to a basal salt nutrient or an aqueous solution of salts and other elements that provide cells with water and certain bulk inorganic ions essential for normal cell metabolism and maintains intra-cellular and/or extra-cellular osmotic balance.

The term “culturing” as used herein incubating and/or passaging cells in an adherent, suspension or 3D culture. As used herein, the term “adherent culture” refers to a cell culture system whereby cells are cultured on a solid surface, which may in turn be coated with an insoluble substrate that may in turn be coated with another surface coat of a substrate, such as those listed below, or any other chemical or biological material that allows the cells to proliferate or be stabilized in culture. The cells may or may not tightly adhere to the solid surface or to the substrate.

The substrate for the adherent culture may be any one or combination of tissue culture treated plastic, polyornithine, laminin, poly-lysine, purified collagen, gelatin, fibronectin, tenascin, vitronectin, entactin, heparin sulfate proteoglycans, poly glycolytic acid (PGA), poly lactic acid (PLA), and poly lactic-glycolic acid (PLGA). In one embodiment, the cells are plated on MATRIGEL®-coated plates. In another embodiment, the cells are plated on fibronectin-coated plates. Cells can be cultured in filter cultures and micromass cultures. In an embodiment, cells are plated onto membrane filters, optionally those that are placed into tissue cultures dishes as part of a transwell system (e.g., MILLIPORE®, ALVATEX®). The substrate could also be a bone scaffold substitute such as CPP (calcium polyphosphate) or other pharmaceutically available scaffolds available (e.g., BioOss).

The term “contacting” or “culturing . . . with” is intended to include incubating the component(s) and the cell/tissue together in vitro (e.g., adding the compound to cells in culture) and the step of “contacting” or “culturing . . . with” can be conducted in any suitable manner. For example, the cells may be treated in adherent culture, in suspension culture, or in 3D culture; the components can be added temporally substantially simultaneously (e.g., together in a cocktail) or sequentially (e.g., within 1 hour, 1 day or more from an addition of a first component). The cells can also be contacted with another agent such as a growth factor or other differentiation agent or environments to stabilize the cells, or to differentiate the cells further and include culturing the cells under conditions known in the art. The population of cells can be enriched using different methods such as methods based on markers such as cell surface markers (e.g., FACS sorting etc.).

The term “expressing” also represented as “+” means, with respect to a cell protein level, detectable protein expression compared to a cell that is not expressing the protein, for example as measured by FACS analysis. The term “fold change” in relation to expression, refers to how much a quantity changes between an original and a subsequent measurement. Fold change is considered to be the ratio between the two quantities, e.g., quantities A and B the fold change of B with respect to A is B/A. For example, a change from 30 to 60 is defined as a fold-change of 2. This is also referred to as a “one-fold increase”. For a given comparison, a positive fold change value indicates an increase of expression, while a negative fold change indicates a decrease in expression. This value is typically reported in logarithmic scale (base 2). For example, log 2 fold change of 1.5 for a specific gene in the “WT vs KO comparison” means that the expression of that gene is increased in WT relative to KO by a multiplicative factor of 2\hat{ }1.5≈2.82.

The term “agonist” means an activator, for example, of a pathway or signaling molecule. An agonist of a molecule can retain substantially the same, or a subset, of the biological activities of the molecule (e.g., nodal). For example, a nodal agonist means a molecule that selectively activates nodal signaling.

The term “inhibitor” means a selective inhibitor, for example, of a pathway or signaling molecule. An inhibitor or antagonist of a molecule (e.g., TGFβ inhibitor) can inhibit one or more of the activities of the naturally occurring form of the molecule. For example, a TGFβ inhibitor is a molecule that selectively inhibits TGFβ signaling.

The term “stem cell” or “undifferentiated cell” refers to a cell which is capable of proliferation, self-renewal and giving rise to more progenitor or precursor cells having the ability to generate a large number of mother cells that can in turn give rise to differentiated, or differentiable, daughter cells. The daughter cells can for example be induced to proliferate and produce progeny cells that subsequently differentiate into one or more mature cell types, while also retaining one or more cells with parental developmental potential. The term “stem cell” includes pluripotent stem cell.

As used herein, the term “differentiation” refers to a phenomenon in which a cell divides and proliferates, and the structure or function of a cell is specialized during the growth of an entire object. In other words, it refers to the process by which cells, tissues, etc. of living organisms change into a form and function suitable for performing the role given to each, etc.) and the process of changing into endoderm cells, as well as the process of changing hematopoietic stem cells into red blood cells, leukocytes, platelets, etc., that is, progenitor cells expressing specific differentiation traits, can all be included in differentiation.

The term “pluripotency” (or pluripotent), as used herein refers to a stem cell that has the potential to differentiate into any of the three germ layers: endoderm (for example, interior stomach lining, gastrointestinal tract, the lungs), mesoderm (for example, muscle, bone, blood, urogenital), or ectoderm (for example, epidermal tissues and nervous system).

The term “Induced pluripotent stem cell” (iPSC), as used herein, is a type of pluripotent stem cell artificially derived from a non-pluripotent cell.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific form or combination of forms of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the forms and does not pose a limitation on the scope of the forms unless otherwise claimed. No language in the specification should be construed as indicating any non-claimed element as essential to the practice of the invention.

Recitation of ranges of values herein are merely intended to serve a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Disclosed are in vitro generated organoids. In some forms, these organoids include a population of differentiated nasal epithelial cells having cellular heterogeneity and molecular markers similar to mature nasal epithelial tissue. In some forms, the population of differentiated nasal epithelial cells are human nasal epithelial cells.

In some forms, the population of differentiated nasal epithelial cells expresses one or more molecular markers selected from the group comprising Angiotensin-converting enzyme-2 (ACE2), Transmembrane serine protease 2 (TMPRSS2), forkhead-box G1 (FOXG1), TUBB4B (Tubulin Beta 4B Class IVb), SIX3, paired box-7 (PAX7), P63, Keratin 5 (CK5), Sentan (SNTN), Mucin 5AC (MUC5AC), MUC15 (Mucin 15, Cell Surface Associated), and CC10. In some forms, the population of differentiated nasal epithelial cells have lower expression of genes selected from the group comprising FOLR1, LMO3, and IRX2 compared to trachea/bronchi cells. In some forms, the population of differentiated nasal epithelial cells have about 0.25-fold, about 0.50-fold, about 0.75-fold, about 1.0-fold, or about 1.25-fold lower expression of FOLR1 compared to trachea/bronchi cells. In some forms, the population of differentiated nasal epithelial cells have about 1.0-fold, about 1.5-fold, about 2.0-fold, about 2.5-fold, or about 2.5-fold lower expression of LMO3 compared to trachea/bronchi cells. In some forms, the population of differentiated nasal epithelial cells have about 0.5-fold, about 1.0-fold, about 1.5-fold, about 2.0-fold, about 2.5-fold, or about 3.0-fold lower expression of IRX2 compared to trachea/bronchi cells.

In some forms, the cellular heterogeneity comprises having a mixture of basal cells, ciliated cells, goblet cells, and club cells. In some forms, the population of differentiated nasal epithelial cells is organized in a three-dimensional structure and is encapsulated within a hydrogel or embedded within a biocompatible scaffold that provides structural support and promotes tissue organization.

In some forms, the population of differentiated nasal epithelial cells comprises basal cells at a percentage of about 12% to about 35%, ciliated cells at a percentage of about 35% to about 45%, goblet cells at a percentage of about 5% to about 10%, and club cells at a percent of about 0.1% to about 5%.

In some forms, ACE2 is expressed by about 20% to about 45% of the population of differentiated nasal epithelial cells, and TMPRSS2 is expressed by about 40% to about 55% of the population of differentiated nasal epithelial cells.

In some forms, the population of differentiated nasal epithelial cells are organized in a two-dimensional (2D) monolayer. In some forms, the population of differentiated nasal epithelial cells are incubated for a period of time during differentiation in a medium having a slightly acidic pH of about 6.0 to about 6.9, preferably about 6.6.

In some forms, the population of differentiated nasal epithelial cells comprise basal cells at a percentage of about 1% to about 10%, ciliated cells at a percentage of about 75% to about 92%, goblet cells at a percentage of about 4% to about 22%, and club cells at a percent of about 0.05% to about 2%.

In some forms, ACE2 is expressed by about 30% to about 50% of the population of differentiated nasal epithelial cells, and TMPRSS2 is expressed by about 85% to about 100% of the population of differentiated nasal epithelial cells.

In some forms, the population of differentiated nasal epithelial cells are derived from undifferentiated nasal cells obtained non-invasively from a subject, wherein the subject is preferably a human. In some forms, the population of differentiated nasal epithelial cells are genetically modified to increase or reduce expression of specific genes involved in nasal tissue development, function, or disease. In some forms, the population of differentiated nasal epithelial cells exhibit functional properties, including mucus production, ciliary beating, and responses to olfactory stimuli.

Also disclosed are methods for generating a three-dimensional (3D) organoid of the nasal epithelium. In some forms, the method includes the steps of:
(i) adhering cells onto a support structure that promotes the formation of (3D) organoids, wherein the cells are nasal turbinate cells capable of differentiating into nasal epithelial cells;
(ii) culturing the nasal turbinate cells on the support structure in an expansion culture medium for a period of time to produce 3D human nasal undifferentiated cells, wherein the period of time is about 8 days to about 12 days, preferably about 10 days;
(iii) culturing the 3D nasal undifferentiated cells on the support structure in a basal medium for period of time to enlarge the central lumen, wherein the period of time is about 3 days to about 5 days, preferably about 4 days;
(iv) culturing the 3D nasal undifferentiated cells on the support structure produced in step (iii) in a proximal differentiation medium for a period of time to generate the 3D differentiated organoid of the human nasal epithelium, wherein the period of time is about 5 days to about 7 days, preferably about 6 days.

In some forms, the expansion culture medium comprises basal media comprising one or more buffering agents, one or more growth factors, a ROCK inhibitor, a TGF β inhibitor, a MAPK inhibitor, a WNT agonist, a BMP-4 inhibitor, one or more supplements, and one or more antibiotics. In some forms, the basal media is Dulbecco's Modified Eagle's Medium (DMEM), Advanced DMEM/F12, RPMI-1640, or Minimum Essential Medium (MEM). In some forms, the buffering agent is HEPES buffer present in an amount ranging from 0.5% to about 1.5%, preferably about 1.0%. In some forms, the one or more growth factors are selected from the group comprising Epidermal growth factor (EGF) and fibroblast growth factor (FGF), wherein the growth factors are preferably FGF-7 in a concentration from about 3 ng/ml to about 7 ng/ml, preferably about 5 ng/ml, and FGF-10 in a concentration from about 15 ng/ml to about 25 ng/ml, preferably about 20 ng/ml. In some forms, the ROCK inhibitor is Y-27632 in a concentration from about 3 μM to about 7 μM, preferably about 5 μM. In some forms, the TGF-β inhibitor is A8301 in a concentration from about 400 nM to about 600 nM, preferably about 500 nM. In some forms, the MAPK inhibitor is SB202190 in a concentration from about 0.5 μM to about 1.5 μM, preferably 1.0 μM. In some forms, the WNT agonist is R Spondin 1 present in an amount from about 8% to about 12%, preferably about 10%. In some forms, R Spondin 1 and/or Noggin are added to the medium as a conditioned medium, which is the medium harvested from stable cell lines expressing R Spondin 1 and Noggin. Percent of an agent in a medium, as used in herein, refers to volume percentage e.g., 10 ml of each conditioned medium can be added to 100 ml basal media to prepare an expansion medium containing 10% of each supplement. Mathematical calculations involved in making solutions are further described in Stephenson, “Calculations for Molecular Biology and Biotechnology”, 3^{rd} Edition, Chapter 2—Solutions, Mixtures, and Media, pages 15-42 (2016), which is incorporated herein in its entirety. In some forms, the BMP-4 inhibitor is Noggin present in a concentration of about 8% to about 12%, preferably about 10%. In some forms, the one or more supplements are selected from the group comprising GlutaMAX® in an amount from about 0.5% to about 1.5%, preferably about 1%, B27 supplement, N-acetylcysteine in concentration from about 1.0 mM to about 1.5 mM, preferably about 1.25 mM, and nicotinamide in concentration from about 7 mM to about 13 mM, preferably about 10 mM. In some forms, the one or more antibiotics are selected from the group comprising Penicillin-Streptomycin in an amount from about 0.5% to about 1.5%, preferably about 1.0%, and Primocin in a concentration from about 75 μg/ml to about 125 μg/ml, preferably about 100 μg/ml.

In some forms, the support structure comprises materials selected from the group comprising Matrigel®, RGD-functionalized PEG hydrogel crosslinked using factor XIIIa, collagen, hyaluronic acid extracellular matrix, synthetic hydrogels, decellularized extracellular matrix, alginate, Poly (lactic-co-glycolic acid) (PLGA), or electrospun fibers. In preferred forms, the support structure comprises Matrigel®.

In some forms, the basal medium is Advanced DMEM/F-12 supplemented with about 1% HEPES buffer, about 1% GlutaMAX®, and about 1% Penicillin-Streptomycin. In some forms, the proximal differentiation medium comprises basal medium supplemented with a γ-secretase inhibitor in a concentration from about 7 μM to about 13 μM, preferably about 10 μM, and wherein the proximal differentiation medium has a pH of about 7.2 to about 7.6, preferably about 7.4.

In some forms, the method further includes replenishing the expansion medium once a day, three times a week, or 4 times a week. In some forms, the method further includes re-plating and passaging 3D human nasal undifferentiated cells one or more times.

Also disclosed are method for deriving a two-dimensional (2D) organoid monolayer of the nasal epithelium from the disclosed 3D nasal undifferentiated cells. In some forms, the method includes the steps of:
(a) dissociating the 3D nasal undifferentiated cells into a plurality of single nasal undifferentiated cells;
(b) transferring the plurality of single nasal undifferentiated cells to a two-chamber support structure and culturing the plurality of single nasal undifferentiated cells in expansion medium for a period of time to allow cell attachment and expansion, wherein the period of time is about 1 day to about 3 days; and
(c) culturing the expanded nasal undifferentiated cells in a first and second proximal differentiation medium for a total of about 8 days to generate confluent two-dimensional (2D) organoid monolayers.

In some forms, the two-chamber support structure comprises a top-chamber and bottom-chamber, and wherein the single nasal undifferentiated cells are adhered to the top chamber of the support structure.

In some forms, the culturing of step (c) includes:
adding the first proximal differentiation medium to the top chamber and the bottom chamber, wherein the first proximal differentiation medium has a pH of about 7.2 to about 7.6, preferably about 7.4;
incubating the nasal undifferentiated cells for about 2 days;
replacing the first proximal differentiation medium in the top chamber with the second proximal differentiation medium, wherein the second proximal differentiation medium has a pH of about 6.4 to about 6.8, preferably about 6.6; and
incubating the nasal undifferentiated cells for about 6 days.

Also disclosed are methods for screening an agent for an effect on the nasal epithelium. In some forms, the method includes contacting in vitro generated organoid as disclosed herein with the agent, and determining the effect of the agent on survival, proliferation, differentiation, morphologic parameters, genetic parameters, or functional parameters of the in vitro generated organoid.

In some forms, the agent is an antiviral agent, antibacterial agent, antifungal agent, or anti-allergenic agent, wherein the effect of the agent is indicative of the agent being safe for the treatment of respiratory infections. In some forms, the agent is nucleic acids or analogs thereof, polypeptides or analogs thereof, antibodies, chemicals, small molecules, and/or any combination thereof.

Also disclosed are kits for generating a two-dimensional monolayer or three-dimensional organoid of the human nasal epithelium. In some forms, the kit includes a combination of two or more of:
(i) Expansion culture medium for deriving the 3D undifferentiated nasal cells comprising basal media comprising one or more of a buffering agent, a growth factor, a ROCK inhibitor, a TGF β inhibitor, a MAPK inhibitor, a WNT agonist, a BMP-4 inhibitor, one or more supplements, and one or more antibiotics;
(ii) Basal media comprising Advanced DMEM/F-12 supplemented with a HEPES buffer, GlutaMAX®, and an antibiotic;
(iii) one or more proximal differentiation media comprising basal medium supplemented with a γ-secretase inhibitor; and
(iv) one or more scaffold or support structures.

### II. Compositions

Robust human nasal organoid culture systems, including two-dimensional and three-dimensional human nasal organoids have been established. The disclosed nasal organoid monolayers are superior to most, if not all, existing respiratory organoid models. The nasal organoids were shown to be stably passaged for more than 6 months, providing a renewable source of nasal epithelial cells. Thus, the nasal organoid culture system provides an unlimited source of physiologically active nasal epithelial cells for studying respiratory pathogens, circumventing the restriction of cultured primary nasal epithelial cells.

The nasal organoids exhibit remarkable cellular heterogeneity and structural complexity, closely resembling the native human nasal epithelium. The disclosed human nasal organoids are suitable for recapitulating nasal tissue complexity for modeling nasal diseases, studying barrier function and drug delivery, high-throughput drug screening, and assessing the effects of pathogens and environmental toxins on the nasal respiratory epithelium.

Although not intended to be limiting, the following are exemplary features of the disclosed nasal organoids: (1) the nasal organoids were and can be derived from a nasal epithelial cells obtained via a nasal swab; (2) the organoids can have long-term expandability (i.e., they can be passaged continuously up to six months or more); (3) the organoids can produce multiple cell types including ciliated cells, basal cells, goblet cells, and club cells; (4) the organoids were and can be extensively characterized via, for example, light microscopy for brightfield images and video of beating cilia, RT-qPCR for cell-type specific markers and compartment-specific markers, IF staining for cell-type identification and formation of tight junction, scanning electron microscopy for ultrastructural images of cilia, TEER for barrier integrity, flow cytometry for cell-type composition and expression of host dependency factor; (5) the organoid types can include 3D dNSO, 2D dNSO (at pH 7.4), and 2D optimized dNsO (pH 6.6/7.4); and (6) replication kinetics of the organoids were and can be extensively characterized via, for example, RT-qPCR and TCID₅₀-~3-4 log 10 unit for ancestral viruses and ~5-6 log 10 unit for Delta/Omicron variants.

### A. Nasal Organoids

As described in the non-limiting Examples, undifferentiated 3D nasal organoids derived from nasal epithelial cells were obtained from a nasal swab of the inferior turbinate of a subject, and induced to generate differentiated 3D and 2D nasal organoids. It was also demonstrated that the differentiated 3D and 2D nasal organoids sustain productive SARS-CoV-2 infection and recapitulated the higher replicative fitness of the Omicron and Delta variants compared to the ancestral virus.

Thus, differentiated 3D and 2D nasal organoids are disclosed. The differentiated 3D and 2D nasal organoids exhibit cellular heterogeneity, molecular composition, structural complexity, and functionality similar to the nasal epithelium of the human nasal cavity.

### 1. Structure of the Nasal Organoids

The disclosed differentiated nasal organoids typically exhibit morphological characteristics and molecular markers similar to mature nasal tissue. The nasal organoids typically contain a population of differentiated nasal epithelial cells, including basal cells, ciliated cells, goblet cells, and club cells. The basal cells are multipotent cells attached at the basal lamina of the nasal epithelium and possess regenerative capabilities, contributing to the continuous renewal of the respiratory lining. Morphologically, basal cells are small, cuboidal cells with a large nucleus, few organelles, and scattered microvilli (Crystal, Am. J. Respir. Crit. Care Med. 190:1355-1362 (2014)). To this end, basal cells are the principal stem cells of the airway, with the ability to self-renew post-injury and differentiate into most other important cell types including goblet, club and ciliated cells, tuft cells, pulmonary neuroendocrine cells (PNECs), and pulmonary ionocytes (Montoro, et al., Nature 560, 319-324 (2018); Hong, et al., Am. J. Physiol. Lung Cell. Mol. Physiol., 286: L643-L649 (2004); Rock, et al. Proc. Natl Acad. Sci. USA 106:12771-12775 (2009); Hajj, et al. Stem Cells, 25, 139-148 (2007)).

The disclosed nasal organoids also contain a subpopulation of ciliated cells. Ciliated cells are columnar epithelial cells that line the luminal surface of the nasal epithelium. Morphologically, ciliated cells are characterized by their elongated shape, polarized organization, and the presence of numerous motile cilia projecting from their apical surface. These cilia are microtubule-based structures that undergo rhythmic, coordinated beating motions. The beating motion of the cilia helps to move mucus and trapped particles out of the respiratory tract (Bustamante-Marin, et al., Cold Spring Harb. Perspect. Biol. 9, a028241 (2017)).

The nasal organoids also contain a subpopulation of goblet cells. Morphologically, goblet cells have a rounded shape and an enlarged apical cytoplasm. The most distinctive feature of goblet cells is the presence of abundant secretory granules within their cytoplasm, which are filled with mucin, the primary component of mucus. Goblet cells play a critical role in maintaining respiratory health and homeostasis through their secretion of mucus (Whitsett, Ann. Am. Thorac. Soc., 15: S143-S148 (2018)). The mucus layer generated by goblet cells serves several vital functions in the nasal epithelium, including but not limited to trapping and clearing foreign particles, and lubricating the airways.

The nasal organoids contain a small subpopulation of club cells, also known as Clara cells. Club cells are non-ciliated, dome-shaped secretory cells have a rounded nucleus and a well-developed endoplasmic reticulum (Boers, et al., Am. J. Respir. Crit. Care Med., 159:1585-1591 (1999)). Club cells are typically characterized by the presence of secretory granules containing various bioactive substances that contribute to the respiratory defense mechanisms and epithelial homeostasis, including but not limited to detoxification enzymes such as cytochrome P450 monooxygenases and glutathione S-transferases, and mucus modulating factors.

The nasal organoids may contain one or more other cell types including but not limited to pulmonary neuroendocrine cells (PNECs), tuft cells, pulmonary ioncytes, and supporting (sustentacular cells). PNECs are innervated epithelial-resident cells that sense airway activity and secrete neuropeptides to stimulate immune responses (Branchfield, et al., Science 351, 707-710 (2016)). Tuft cells (also known as brush cells), are columnar epithelial cells having an elongated shape, with a single apical tuft of microvilli, giving them their name. The tuft of microvilli increases the cell surface area, allowing for increased sensory and signaling interactions with the surrounding environment. Tuft cells contain a centrally located nucleus and abundant organelles involved in protein synthesis and secretion (Schneider, et al., Nat. Rev. Immunol. 19, 584-593 (2019)). The cell types comprising the nasal epithelium are further described in the following research and review articles: Scherzad et al., J Inflamm Res., 12:309-317 (2019); Harkema et al., Toxicologic Pathology, 34 (3): 252-269 (2006); and Davis and Wypych, Mucosal Immunology, 14:978-990 (2021); all of which are incorporated herein in their entireties.

The nasal organoids typically have a molecular architecture similar to the native nasal epithelium. The nasal organoids may express one or more genes selected from the group containing Angiotensin-converting enzyme-2 (ACE2), Transmembrane serine protease 2 (TMPRSS2), forkhead-box G1 (FOXG1), SIX3, paired box-7 (PAX7), P63, Keratin 5 (CK5), Sentan (SNTN), Mucin 5AC (MUC5AC), TUBB4B (Tubulin Beta 4B Class IVb), MUC15 (Mucin 15, Cell Surface Associated), OTX1/2, and CC10. In some forms, the nasal organoids may express one or more cell type markers for basal cells including but not limited to P63, CK1, CK5, CK10, CK14, and DLK2. In some forms, the nasal organoids may express one or more cell type markers for ciliated cells including but not limited to LYPD2 (LY6/PLAUR Domain Containing 2), SPRR3 (Small Proline-Rich Protein 3), C15orf48 (Chromosome 15 Open Reading Frame 48), FOXJ1, ACCTUB, and SNTN. In some forms, the nasal organoids may express one or more cell type markers for secretory goblet cells including but not limited to LYNX1, S100A4, CEACAM5, LYPD2, PI3, and MUC4.

In some forms, the nasal organoids may express one or more cell type markers for ionocytes including but not limited to ASCL3 and CFTR. In some forms, the nasal organoids may express one or more cell type markers for ionocyte regulatory units, including but not limited to ASCL3, FOXI1, and DMRT2. In preferred forms, the nasal organoids express lower levels of trachea/bronchi associated genes, including but not limited to FOLR1, LMO3, and IRX2. In some forms, the population of differentiated nasal epithelial cells have about 0.25-fold, about 0.50-fold, about 0.75-fold, about 1.0-fold, or about 1.25-fold lower expression of FOLR1 compared to trachea/bronchi cells. In some forms, the population of differentiated nasal epithelial cells have about 1.0-fold, about 1.5-fold, about 2.0-fold, about 2.5-fold, or about 2.5-fold lower expression of LMO3 compared to trachea/bronchi cells. In some forms, the population of differentiated nasal epithelial cells have about 0.5-fold, about 1.0-fold, about 1.5-fold, about 2.0-fold, about 2.5-fold, or about 3.0-fold lower expression of IRX2 compared to trachea/bronchi cells.

In some forms the nasal organoids may express one or more genes associated with viral cells, such as coronavirus cells, including but not limited to ACE2 and TMPRSS2. In some forms, the nasal organoids may express one or more genes encoding regulatory units e.g., MESP1, (Mesoderm Posterior BHLH Transcription Factor 1) (Takahashi et al., Development, 2005; 132:787-796). In some forms, the nasal organoids may express one or more genes encoding transcription factors related to IFN pathways including but not limited to IRF1, IFI27, and STAT1.

i. Organization of the 3D Nasal Organoids

An array of conditions and differentiation protocols were tested to generate 3D differentiated nasal organoids with similar morphology and function to the mature nasal epithelium. Thus, in some forms, the population of differentiated nasal epithelial cells are organized in a three-dimensional structure, thereby producing a differentiated 3D nasal organoid. The 3D nasal organoids generally have multiple airway epithelial cell types in proportions that are distinct from 3D undifferentiated organoids and organoids derived from trachea/bronchioles.

In some forms, the 3D nasal organoids contain basal cells in a percentage from about 12% to about 35% of the total cells forming the 3D nasal organoid. In some forms, the basal cells account for about 12%, about 15%, about 20%, about 25%, about 30%, or about 35% of the total cells forming the 3D nasal organoid. In preferred forms, the basal cells account for about 23% of the total cells forming the nasal organoid.

In some forms, the 3D nasal organoids contain ciliated cells in a percentage from about 35% to about 45% of the total cells forming the 3D nasal organoid. In some forms, the ciliated cells account for about 36%, about 37%, about 38%, about 39%, about 40%, about 41%, about 42%, about 43%, about 44%, or about 45% of the total cells forming the 3D nasal organoid. In preferred forms, the ciliated cells account for about 40% of the total cells forming the nasal organoid.

In some forms, the 3D nasal organoids contain goblet cells in a percentage from about 5% to about 10% of the total cells forming the 3D nasal organoid. In some forms, the secretory goblet cells account for about 5%, about 6%, about 7%, about 8%, about 9%, or about 10% of the total cells forming the 3D nasal organoid. In preferred forms, the goblet cells account for about 8% of the total cells forming the nasal organoid.

In some forms, the 3D nasal organoids contain club cells in a percentage from about 0.1% to about 5% of the total cells forming the 3D nasal organoid. In some forms, the secretory club cells account for about 0.1%, about 0.5%, about 1%, about 2%, about 3%, about 4%, or about 5% of the total cells forming the 3D nasal organoid. In preferred forms, the club cells account for about 0.5% of the total cells forming the nasal organoid.

The differentiated 3D nasal organoids typically have a molecular composition similar to the native nasal epithelium as described in Section II(A)(1) above. In some forms, the differentiated 3D nasal organoids may express one or more genes associated with viral cells, such as coronavirus cells, including but not limited to ACE2 and TMPRSS2.

In some forms, ACE2 is expressed by about 20% to about 45% of the population of differentiated nasal cells of the 3D nasal organoid. In some forms, ACE2 is expressed by about 20%, about 25%, about 30%, about 35%, about 40%, or about 45% of the population of differentiated nasal cells of the 3D nasal organoid. Preferably, ACE2 is expressed by about 35% of the population of differentiated nasal cells of the 3D nasal organoid.

In some forms, TMPRSS2 is expressed by about 40% to about 55% of the population of differentiated nasal cells of the 3D nasal organoid. In some forms, TMPRSS2 is expressed by about 40%, about 42%, about 44%, about 46%, about 48%, about 50%, about 52%, about 54%, or about 55% of the population of differentiated nasal cells of the 3D nasal organoid. Preferably, TMPRSS2 is expressed by about 48% of the population of differentiated nasal cells of the 3D nasal organoid.

ii. Organization of the 2D Nasal Organoid Monolayers

As described in the non-limiting Examples, an array of conditions was tested to generate optimized nasal organoid monolayers, which better mimic nasal epithelium exposure to invading pathogens. A critical feature of the disclosed optimized 2D nasal organoid monolayers lies in the partial incubation of the undifferentiated nasal cells in a differentiation medium having a slightly acidic pH that is more representative of the physiological milieu of the surface liquid in the human airways, including the nasal mucosa. The methods for making the optimized 2D nasal organoid monolayers are described in detail in Section III below.

Thus, in some forms, the population of differentiated nasal epithelial cells are organized in a two-dimensional (2D) monolayer, thereby producing a differentiated 2D nasal organoid. Similar to the differentiated 3D nasal organoids, the 2D nasal organoid monolayers generally have multiple airway epithelial cell types in proportions that are distinct 3D undifferentiated organoids and organoids derived from trachea/bronchioles.

In some forms, the 2D nasal organoid monolayer contains basal cells in a percentage from about 1% to about 10% of the total cells forming the 2D nasal organoid monolayer. In some forms, the basal cells account for about 1%, about 2%, about 4%, about 6%, about 8%, or about 10% of the total cells forming the 2D nasal organoid monolayers. In preferred forms, the basal cells account for about 4% of the total cells forming the 2D nasal organoid monolayer.

In some forms, the 2D nasal organoid monolayer contains ciliated cells in a percentage from about 75% to about 95% of the total cells forming the 2D nasal organoid monolayers. In some forms, the ciliated cells account for about 75%, about 80%, about 85%, about 90%, or about 95% of the total cells forming the 2D nasal organoid monolayer. In preferred forms, the ciliated cells account for about 90% of the total cells forming the 2D nasal organoid monolayer.

In some forms, the 2D nasal organoid monolayer contains goblet cells in a percentage from about 4% to about 24% of the total cells forming the 2D nasal organoid monolayers. In some forms, the secretory goblet cells account for about 4%, about 8%, about 12%, about 16%, about 20%, or about 24% of the total cells forming the 2D nasal organoid monolayer. In preferred forms, the goblet cells account for about 18% of the total cells forming the 2D nasal organoid monolayers.

In some forms, the 2D nasal organoid monolayer contain club cells in a percentage from about 0.05% to about 2% of the total cells forming the 2D nasal organoid monolayers. In some forms, the secretory club cells account for about 0.5%, about 1.0%, about 1.5%, or about 2% of the total cells forming the 2D nasal organoid monolayer. In preferred forms, the club cells account for about 0.5% of the total cells forming the 2D nasal organoid monolayer.

The differentiated 2D nasal organoid monolayer typically has a molecular composition similar to the native nasal epithelium as described in Section II (A) (1) above. In some forms, the differentiated 2D nasal organoid monolayers may express one or more genes associated with viral cells, such as coronavirus cells, including but not limited to ACE2 and TMPRSS2.

In some forms, ACE2 is expressed by about 30% to about 55% of the population of differentiated nasal cells of the 2D nasal organoid monolayer. In some forms, ACE2 is expressed by about 30%, about 35%, about 40%, about 45%, about 50%, or about 55% of the population of differentiated nasal cells of the 2D nasal organoid monolayer. Preferably, ACE2 is expressed by about 45% of the population of differentiated nasal cells of the 2D nasal organoid monolayers.

In some forms, TMPRSS2 is expressed by about 85% to about 100% of the population of differentiated nasal cells of the 2D nasal organoid monolayers. In some forms, TMPRSS2 is expressed by about 85%, about 90%, about 95%, or about 100% of the population of differentiated nasal cells of the 2D nasal organoid monolayer. Preferably, TMPRSS2 is expressed by about 95% of the population of differentiated nasal cells of the 2D nasal organoid monolayer.

### 2. Function of the 2D and 3D Nasal Organoids

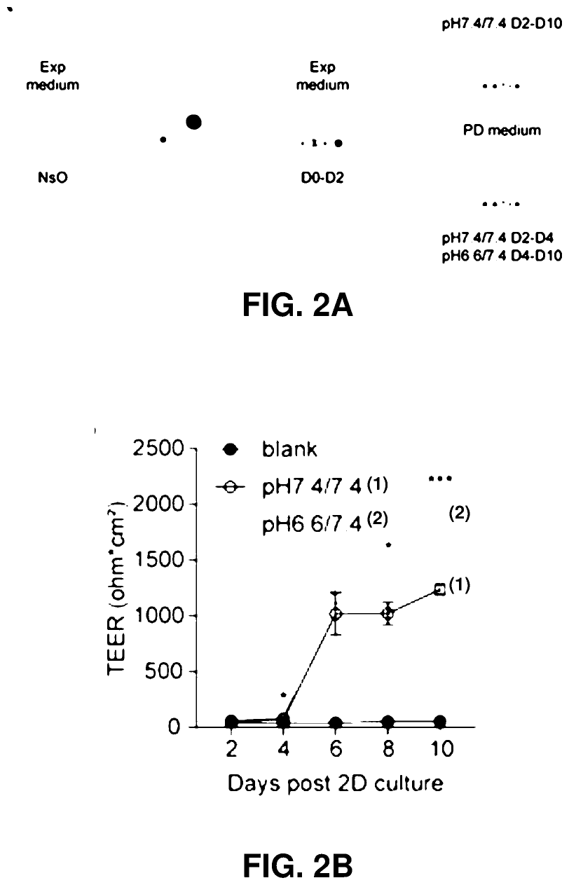

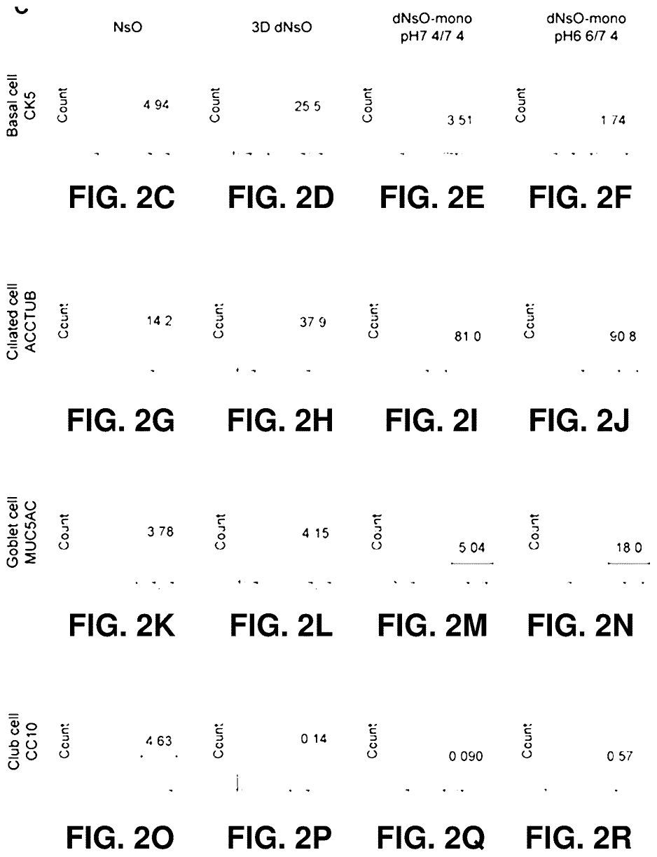

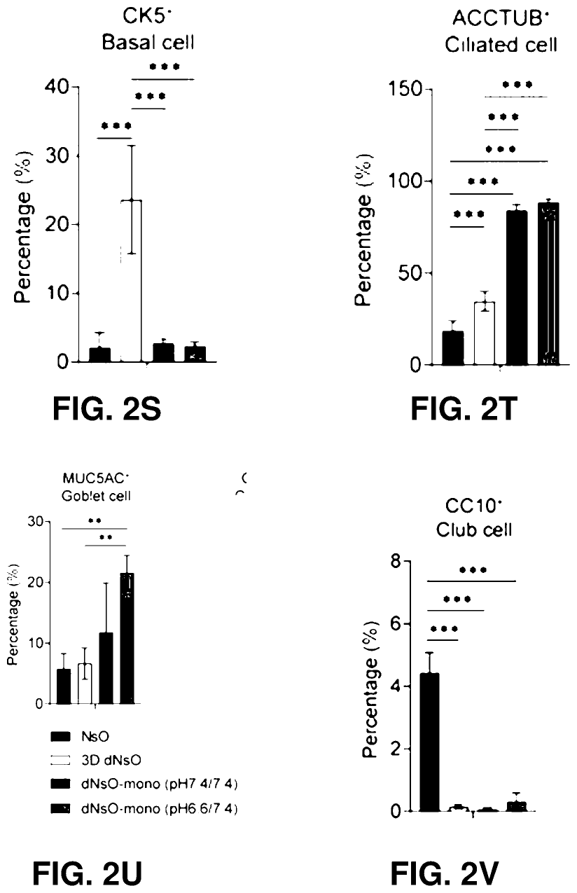

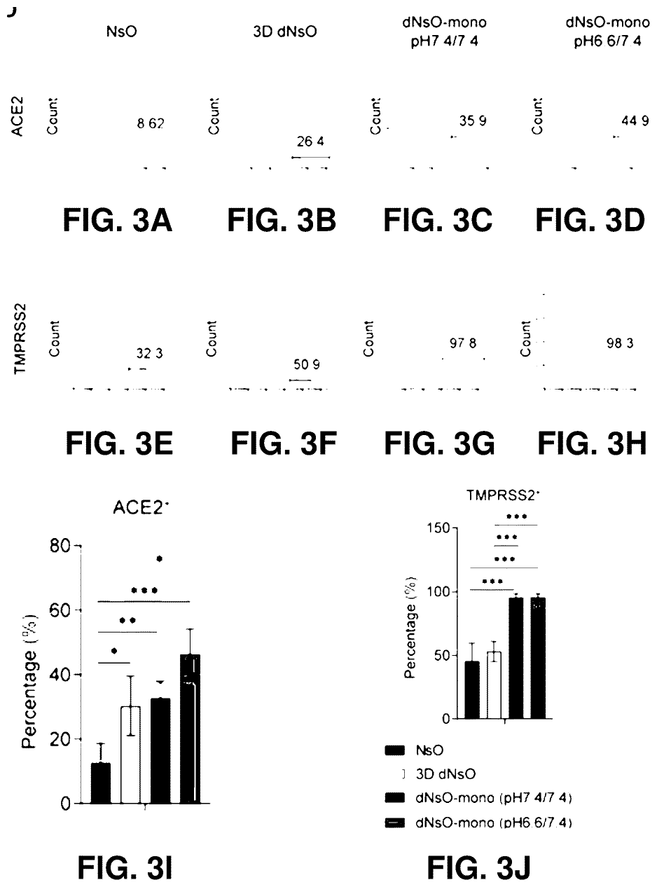

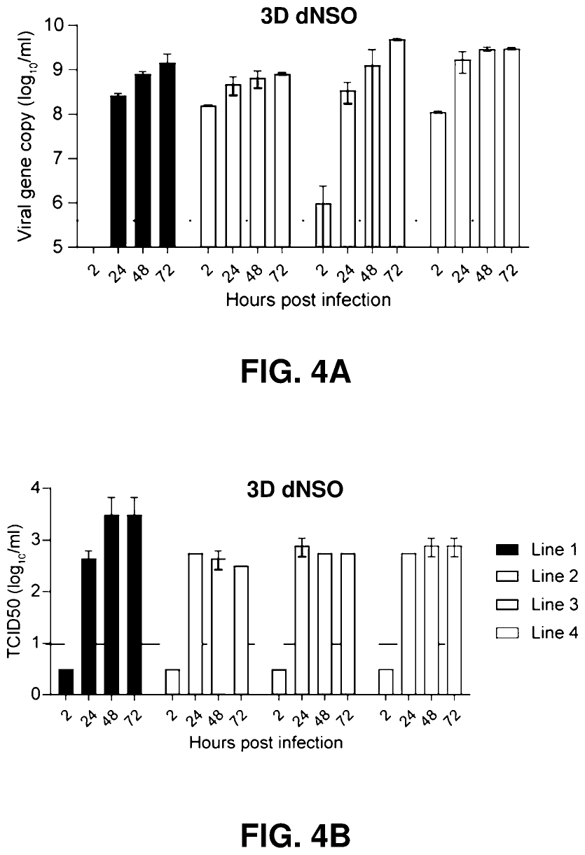

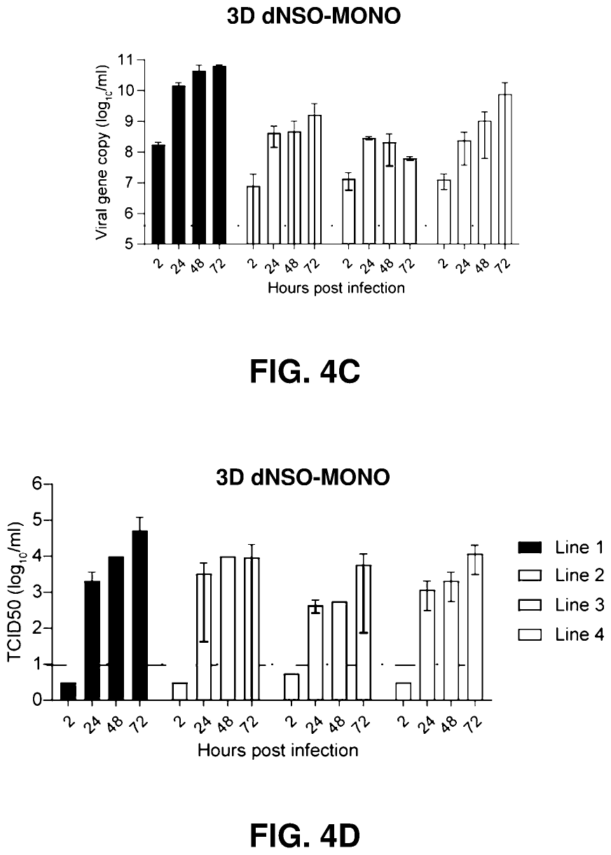

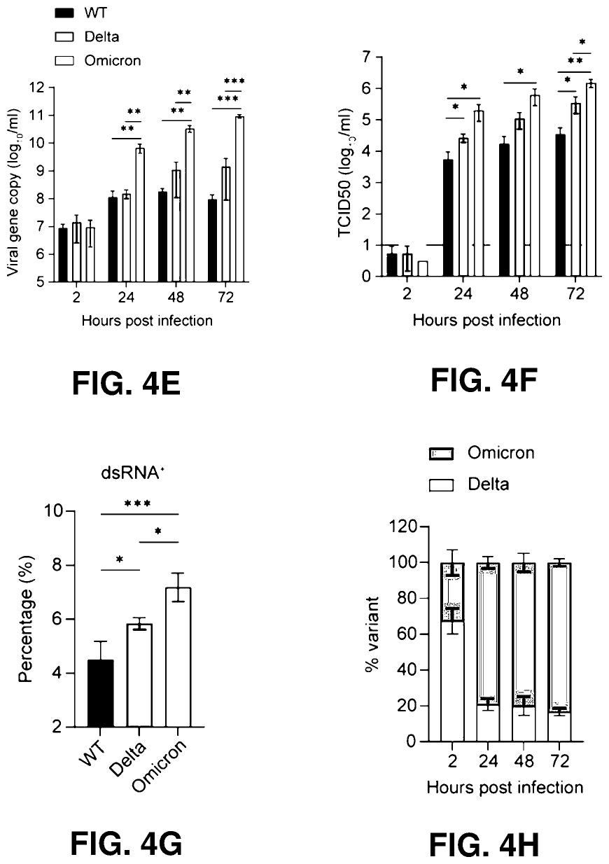

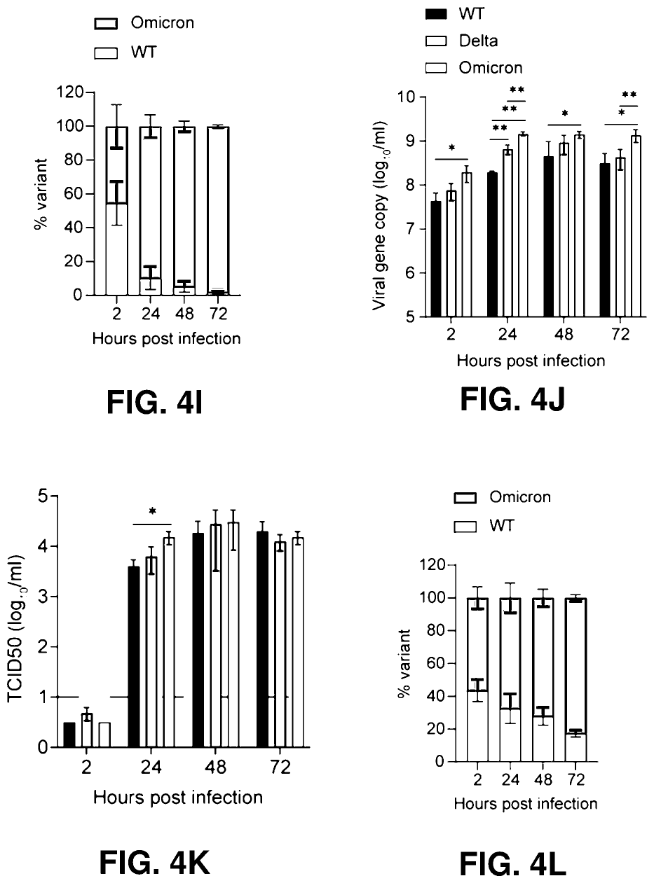

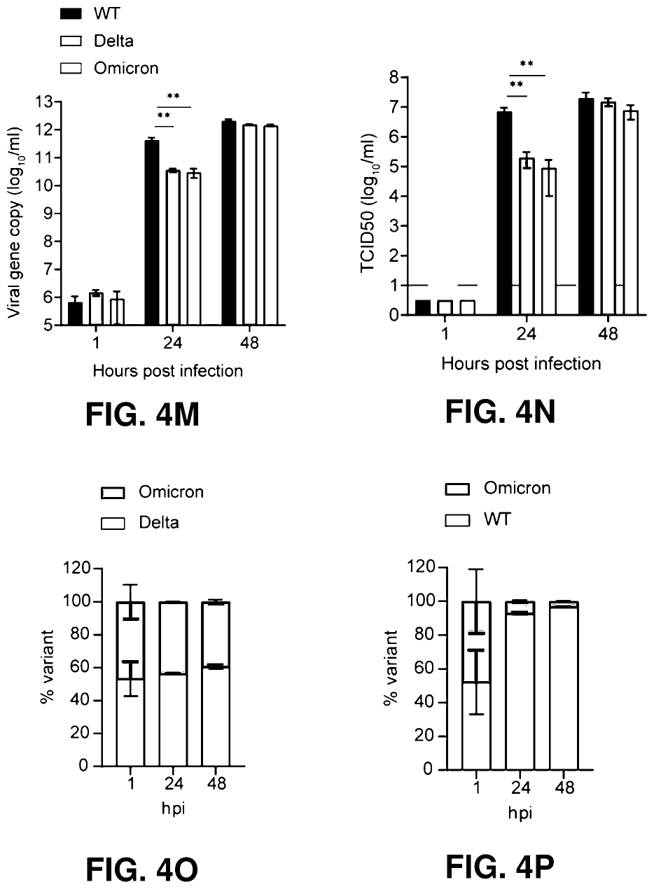

The disclosed 2D and 3D nasal organoids recapitulates respiratory functions similar to that of the native nasal epithelium, thereby providing an improved research platform to investigate various aspects of respiratory physiology. For example, as described in the non-limiting Examples, organoid monolayers induced differentiation with slightly acidic medium (pH 6.6) in the top chamber displayed a more intensified epithelial barrier (FIG. 2B) and a higher expression of ACE2, the cellular receptor for SARS-CoV-2 infection (FIGS. 3A-3J). Notably, the optimized nasal organoid monolayers accurately reproduce the higher infectivity and elevated replicative fitness of the Omicron variant than prior circulating variants by flow cytometry analysis of infection rate, viral titration detection of viral propagation and the competition assay (FIGS. 4E-41).

Generally, the different cell types forming the 2D and 3D nasal organoids mimic a variety of functions that contribute to respiratory defense, sensory perception, and airway maintenance. In some forms, the 2D and 3D nasal organoids may exhibit filtration and defense functions. In some forms, the goblet cells can produce mucus and the coordinated movement of cilia on the surface of the epithelial cells can mimic native respiratory physiology of removing trapped particles and facilitating their clearance.

In some forms, the 2D and 3D nasal organoids may demonstrate humidity and temperature control. The moist mucous layer on the epithelial surface adds moisture to the air, preventing excessive drying of the respiratory tissues. This humidification process is crucial for maintaining optimal conditions for the proper functioning of the respiratory system. In some forms, the goblet cells in the 3D and 2D nasal organoids produce and secrete mucus, a viscous fluid that helps lubricate the nasal passages and trap inhaled particles and pathogens. In some forms, the mucus also contains antimicrobial components, such as antibodies and enzymes, that aid in the defense against infections.

In some forms, the 2D and 3D nasal organoids may mimic immune physiology of the nasal epithelium. In some forms, the 2D and 3D nasal organoids can contain immune cells, including mast cells, macrophages, and dendritic cells, which actively monitor the nasal environment for potential pathogens or harmful substances. In some forms, immune cells can be included in the 2D and 3D nasal organoids to mimic initiation of immune responses, release of inflammatory mediators or presentation of antigens to activate the immune system processes that facilitate protection against infections or allergic reactions.

In some forms, the 2D organoids may mimic barrier function of the nasal epithelium. The acts 2D organoids may demonstrate barrier function against pathogens, toxins, and irritants. Tight junctions between epithelial cells create a physical barrier that restricts the entry of harmful substances into the underlying tissues. Additionally, the epithelium produces antimicrobial peptides and immunoglobulins, further enhancing its protective function.

In some forms, the 2D nasal organoids contain apical junctional complexes (AJCs), including tight junctions, adherens junctions, desmosomes, and hemidesmosomes, which connect epithelial cells to one another to form a nasal epithelial barrier. In native nasal epithelial tissue, tight junctions are the most apically located epithelial junctions composed of over 40 proteins either as transmembrane proteins or cytoplasmic actin-binding proteins (Campbell et al., Exp Cell Res (2017) 358:39-44). Tight junctions regulate homeostasis of ions, water and certain macromolecules (Georas et al., J Allergy Clin Immunol (2014) 134:509-20; Georas et al., J Allergy Clin Immunol (2014) 134:509-20). Thus, tight junctions are crucial in producing rate-limiting barrier to inhaled pathogens. In some forms, the tight junctions in the 2D nasal organoids include transmembrane proteins such as occludin (OCLN), claudin (CLDN) and junctional adhesion molecules (JAMs). In some forms, the tight junctions contain one or more barrier forming CLDN proteins e.g., CLDN1, CLDN3, CLDN4 and CLDN7.

Adherens junctions are essential for cell adhesion, cell proliferation and differentiation (Bruser et al., Cold Spring Harb Perspect Biol (2017) 9; Campbell et al., Exp Cell Res (2017) 358:39-44). Desmosomes are in close connectivity with adherens junctions, and they play key roles in maintaining intercellular cohesion and cellular integrity (Rubsam et al., Cold Spring Harb Perspect Biol (2018) 10; Hatzfeld et al., Cold Spring Harb Perspect Biol (2017) 9). Hemidesmosomes are responsible to facilitate the stable adhesion of the basal epithelial cells to the basement membrane, and to link the extracellular matrix to the intermediate filaments of the actin cytoskeleton. Nasal barrier integrity and tight junctions are further described in Husna et al., Front Immunol., 12:663626 (2021); Zhang et al., European Journal of Allergy and Clinical Immunology, 71 (3): 295-307 (2016); Laulajainen-Hongisto et al., Front. Cell Dev. Biol., 8:204 (2020); all of which are incorporated herein in their entireties.

### C. Formulations

Formulations containing the 2D nasal organoid monolayers and 3D nasal organoids are also provided. In some forms, the formulation includes 2D or 3D nasal organoids provided in a cell culture media. In some forms, the number of cells in the formulation is between about 1 and 100 million cells, between about 5 and 50 million cells, or preferably about 5 million cells.

In some forms, the formulation contains 3D nasal organoids surrounded by an extracellular matrix (ECM). The injectable material could contain biomaterials such as collagens, polyglycolic acid (pga), polylactic acid, alginates (for example, the calcium salt), polyethylene oxide, fibrin adhesive, polylactic acid-polyglycolic acid copolymer, proteoglycans, glycosaminoglycans, natural biomaterials such as matrigel.

In some forms, the formulation includes 2D nasal organoid monolayers and 3D nasal organoids provided in a cryopreservative. In some forms, the number of cells in the formulation is between about 1 and 30 million cells, preferably 10 million cells.

Media for preservation of cells are known in the art, for example, CRYO-GOLD™ (cryopreservation medium) and CROSSTOR® (cyropreservation freeze media) designed to mitigate temperature-induced molecular cell stress responses during freezing and thawing. All CROSSTOR® products are pre-formulated with USP grade DMSO, a permeant solute cryoprotective agent which helps mitigate damage from the formation of intracellular ice. CROSSTOR® is offered in several packages and pre-formulated with DMSO in final concentrations of 2%, 5%, and 10%. A preferred medium for cell preservation includes 5-10% DMSO, for example, CRYOSTOR® CS10 (a uniquely formulated serum-free, animal component-free, and defined cryopreservation medium containing 10% dimethyl sulfoxide (DMSO)). Additionally, cryoprotectants/cryoprotectant additives which can be include in a cell composition (for cryopreservation) are known in the art and include, ethylene glycol (EG), antioxidants such as taurine, Metformin, gamma amino butyric acid (GABA). Cells may be suspended in a “freeze medium” such as cell culture medium containing 15-20% fetal bovine serum (FBS) and 7-10% DMSO, with or without 5-10% glycerol, at a density, for example, of about 1-10×10⁶ cells/ml. The cells are dispensed into glass or plastic vials, which are then sealed and transferred to a freezing chamber of a programmable or passive freezer. The optimal rate of freezing may be determined empirically. For example, a freezing program that gives a change in temperature of −1° C./min through the heat of fusion may be used. Once vials containing the cells have reached −80° C., they are transferred to a liquid nitrogen storage area.

### D. Kits

The materials described above as well as other materials can be packaged together in any suitable combination as a kit useful for performing, or aiding in the performance of, the disclosed method. It is useful if the kit components in a given kit are designed and adapted for use together in the disclosed method.

In some forms, the kit may be a cell culture kit. In some forms, the cell culture kit may include one or more of undifferentiated 3D nasal organoids, culture reagents, and materials suitable for culturing the 3D and 2D differentiated nasal organoids.

In an exemplary form, a kit for generating a two-dimensional monolayer or three-dimensional organoid of the human nasal epithelium, the kit can contain a combination of two or more of:
(i) Expansion culture medium for deriving the 3D undifferentiated nasal cells containing basal medium. The basal medium can contain one or more of a buffering agent, growth factor, ROCK inhibitor, a TGF β inhibitor, a MAPK inhibitor, a WNT agonist, a BMP-4 inhibitor, one or more supplements, and one or more antibiotics;
(ii) Basal media containing Advanced DMEM/F-12 supplemented with a HEPES buffer, GlutaMAX®, and an antibiotic;
(iii) one or more proximal differentiation media containing basal medium supplemented with a γ-secretase inhibitor; and
(iv) one or more scaffold or support structures.

In some forms, the one or more culture media in the kit may be formulated in deionized, distilled water. In some forms, the one or more culture media will typically be sterilized prior to use to prevent contamination, e.g., by ultraviolet light, heating, irradiation, or filtration. In some forms, the one or more culture media may be frozen (for example, at −20° C. or −80° C.) for storage or transport. In some forms, the one or more culture media may contain one or more antibiotics to prevent contamination.

In some forms, the one or more culture media in the kit may be formulated in deionized, distilled water. In some forms, the one or more culture media will typically be sterilized prior to use to prevent contamination, e.g., by ultraviolet light, heating, irradiation, or filtration. In some forms, the one or more culture media may be frozen (for example, at −20° C. or −80° C.) for storage or transport. In some forms, the one or more culture media may contain one or more antibiotics to prevent contamination, for example, an aqueous saline solution, an aqueous buffer, or a culture medium.

The one or more media in the kit may be contained in hermetically sealed vessels which prevent contamination. Hermetically sealed vessels may be preferred for transport or storage of the culture media. The vessel may be any suitable vessel, such as a flask, a plate, a bottle, a jar, a vial, or a bag.

The kit may also include instructions for use, e.g., for using the expansion medium for deriving the undifferentiated 3D nasal organoids and/or for adjusting the pH of the PD medium to derive the 2D nasal monolayer organoids.

### II. Methods of Making Nasal Organoids

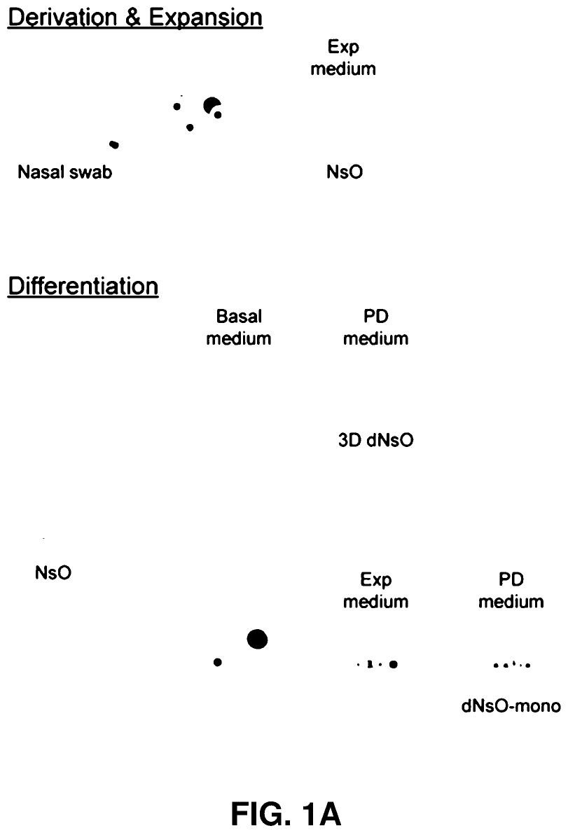

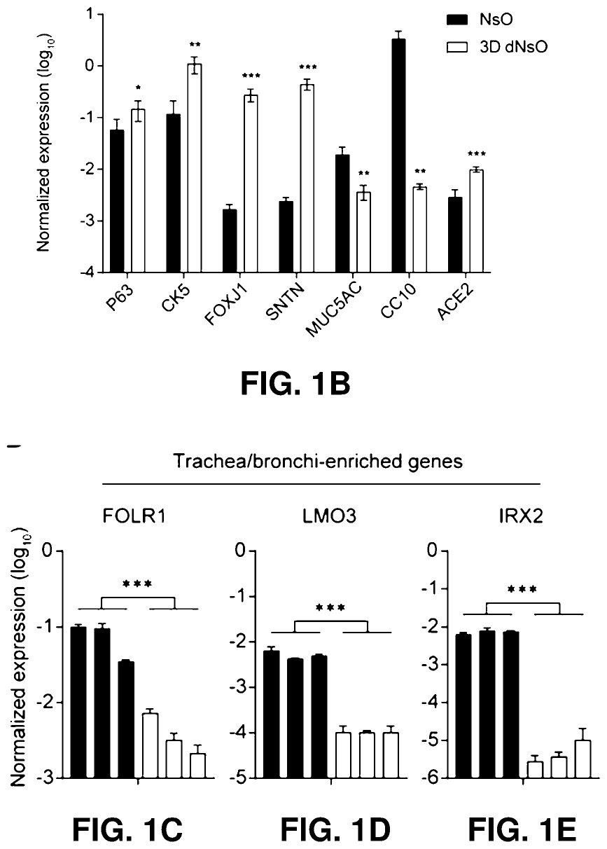

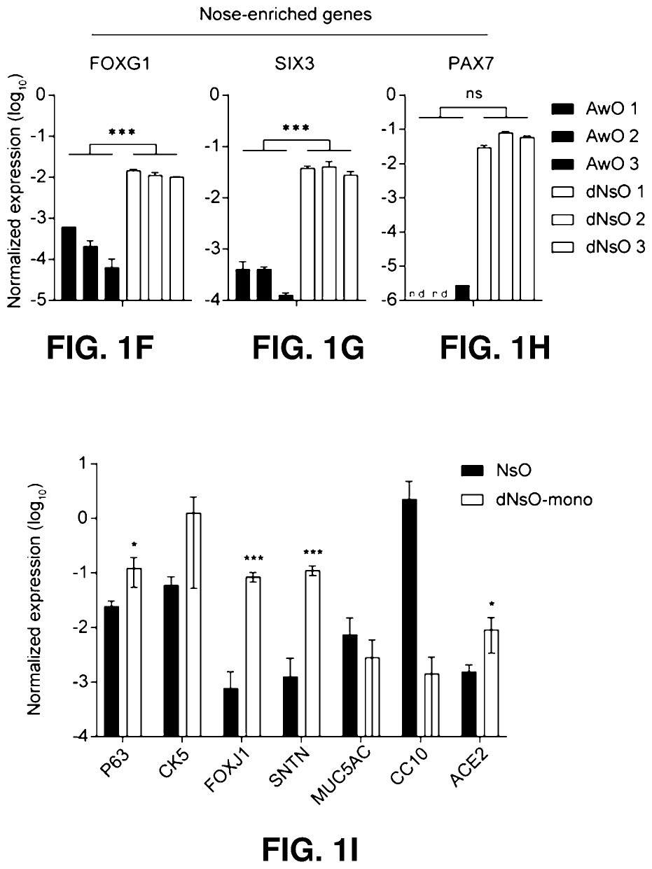

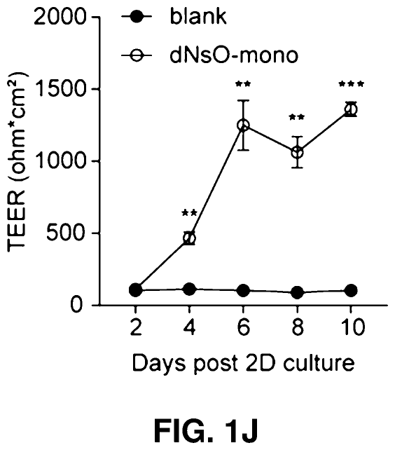

Methods of making the 2D and 3D organoids are also provided. The disclosed methods outline steps for culturing cells obtained non-invasively from nasal turbinate. The methods generally require a two-phase culture system, i.e., expansion and differentiation culture (FIG. 1A). The expansion medium sustained long-term expansion of nasal organoids for more than 6 months by directing the 3D undifferentiated organoids toward an immature state. The differentiation protocols were defined to generate differentiated 3D nasal organoids and organoid monolayers (FIGS. 1B and 1I). The differentiated nasal organoids adequately simulate the native nasal epithelium. Namely, the nasal organoid culture system enables a highly efficient reconstruction and stable expansion of the human nasal epithelium in culture plates for various experimental applications.

The 2D and 3D organoids disclosed herein, once established, are stable for up to one month, two months, or longer, while displaying remarkable phenotypic and genotype stability. They thus overcome the reproducibility and availability limitations of the current in vitro model systems. Several lines of 2D and 3D organoids were obtained from nasal turbinate of healthy patients. These 2D and 3D organoids, include the four major types of airway epithelial cells, i.e., ciliated cell (ACCTUB+ or FOXJ1+), basal cell (P63+), goblet cell (MUC5AC+), and Club cell (CC10+) (FIG. 2C and FIG. 2D). In some forms, generating a line of 3D organoids or 2D monolayer organoids from nasal turbinate cells using the disclosed methods takes between about one week and about four weeks, more preferably, between about 2 weeks and about 3 weeks.

### 1. Sources of Cells

A significant advance is the cell source for deriving nasal organoids. Nasal epithelial cells were procured from the inferior turbinate of healthy donors using a flocked swab, which is a non-invasive procedure, unlike deriving lung organoids using cells from bronchoalveolar lavage or resected tissues that require the invasive manipulations of physicians and surgeons. More importantly, prior studies of human intestinal organoids, the first adult stem cell-derived organoid model, revealed that the epithelial organoids generated from different intestinal segments retain the structural and functional characteristics from which they are derived (29), since tissue identity is imprinted in the adult stem cells and maintained in these organoids during long-term expansion culture (37). Thus, nasal epithelial cells would be an optimal source for deriving physiologically relevant nasal organoids. Indeed, nasal organoids exhibited a compartment-specific gene expression profile (FIGS. 3A-3J). More importantly, nasal organoids retain the genotypic and phenotypic characteristics of original cells, which allows the generation of personalized nasal organoids and provides a facile and robust in vitro model of upper respiratory epithelium for various applications of personalized medicine.

Interestingly, as described in the non-limiting Examples, the nasal organoids have been derived with a great success rate, which was beyond expectations. Presumably, the reason for this superior efficiency may be harvesting cells from healthy individuals and COVID-19 patients via nasal brushing, in which about 60% are ciliated and goblet cells, whereas suprabasal and club cells accounted for approximately 30% of the cells (5). Suprabasal cells are an intermediate cell population between basal and club cells (6). Basal/suprabasal and club cells were reported to be progenitor cells for respiratory epithelial regeneration (38-40). These progenitor cells have clonal expansion capacity in vitro and proliferate to form organoids (40-42). The non-limiting Examples demonstrate that the expansion medium supplemented with R-spondin, Noggin and FGF sustained the clonogenicity of these progenitor cells and enables a consecutive expansion of undifferentiated nasal organoids for approximately 6 months. The facile cell procurement procedure circumvented the relatively shorter expandability of nasal organoids, highlighting nasal organoids as a robust and personalized in vitro model of the nasal epithelium.

The disclosed 2D and 3D organoids can be cultured from a tissue sample preferably a nasal turbinate sample obtained from a mammal, such as any mammal including but not limited to bovine, ovine, porcine, canine, feline, equine, primate. In preferred forms, the tissue sample is obtained from a human. In some forms form, the nasal turbinate cells are obtained from a juvenile or adult human.

In some forms, single cells are obtained from a nasal turbinate sample using a combination of steps that result in single cells. The nasal sample may be aggregated in mucus upon collection. Nasal cells may be isolated by disaggregating the nasal turbinate sample that is to serve as the cell source using techniques known to those skilled in the art. For example, the nasal turbinate sample can be disaggregated mechanically and treated with digestive enzymes and/or chelating agents to release the cells, to form a suspension of individual cells. Enzymatic dissociation can be accomplished by mechanical dissociation with one or more enzymes such as trypsin, chymotrypsin, collagenase, elastase, and/or hyaluronidase, DNase, pronase, dispase etc.

Isolated cells are further cultured as discussed herein. A preferred cell culture medium is a defined synthetic medium, buffered at a pH of 7.4 (preferably between 7.2 and 7.6 or at least 7.2 and not higher than 7.6) with a carbonate-based buffer, while the cells are cultured in an atmosphere comprising between 5% and 10% CO₂, or at least 5% and not more than 10% CO₂, preferably 5% CO₂.

### 2. Expansion Medium

The cells are cultured in supplemented basal cell culture media. Generally, the expansion culture medium contains basal media comprising one or more buffering agents, one or more growth factors, a ROCK inhibitor, a TGFβ inhibitor, a MAPK inhibitor, a WNT agonist, a BMP-4 inhibitor, one or more supplements, and one or more antibiotics. In some forms, the expansion culture medium is base medium supplemented with agents such as Rspondin (a Wnt agonist), a BMP inhibitor, a TGF-beta inhibitor, a fibroblast growth factor (FGF) and Nicotinamide.

In some forms, a base media may include at least one carbohydrate as an energy source and/or a buffering system to maintain the medium within the physiological pH range. Examples of commercially available base media may include, but are not limited to phosphate buffered saline (PBS), Dulbecco's Modified Eagle's Medium (DMEM), Minimal Essential Medium (MEM), Basal Medium Eagle (BME), Roswell Park Memorial Institute Medium (RPMI) 1640, MCDB 131, Click's medium, McCoy's 5 A Medium, Medium 199, William's Medium E, insect media such as Grace's medium, Ham's Nutrient mixture F-10 (Ham's F-10), Ham's F-12, a-Minimal Essential Medium (aMEM), Glasgow's Minimal Essential Medium (G-MEM) and Iscove's Modified Dulbecco's Medium. A preferred basal cell culture medium is selected from DMEM/F12 and RPMI 1640. In a further preferred embodiment, Advanced DMEM/F12 or Advanced RPMI is used, which is optimized for serum free culture and already includes insulin. In this case, the Advanced DMEM/F 12 or Advanced RPMI medium is preferably supplemented with glutamine and Penicillin/streptomycin. In preferred forms, the basal medium comprises Gastrin. In some forms, the basal medium also comprises NAc and/or B27. In some forms, the buffering agent is HEPES buffer present in an amount ranging from 0.5% to about 1.5%, preferably about 1.0%.

In some forms, an expansion medium as described in WO2016/083613 can be used. In a particularly preferred form, an expansion culture medium as outlined in Table 1 is used, which is supplemented base media suitable to maintain 3D undifferentiated organoids in culture.

In some forms, the supplemented basal culture medium used to culture cells dissociated from a nasal turbinate sample does not include a GSK3 inhibitor, for example CHIR99021 (6-[[2-[[4-(2,4-Dichlorophenyl)-5-(5-methyl-1H-imidazol-2-yl)-2-pyrimidinyl]amino]ethyl]amino]-3-pyridinecarbonitrile). Known GSK-inhibitors comprise small-interfering RNAs, 6-Bromoindirubin-30-acetoxime.

A preferred expansion medium is shown in Table 1.

TABLE 1
Composition of the Expansion Medium

| | | | Working | |
|---|---|---|---|---|
| Reagents | Company | Catalog No. | concentration | |
| Advanced DMEM/F12 | Gibco | 12634010 | — | |
| HEPES | Gibco | 15630056 | 1% | |
| GlutaMAX | Gibco | 35050061 | 1% | |
| P/S | Gibco | 15140122 | 1% | |
| Rspondin1 | — | — | 10% | |
| (conditioned medium) | | | | |
| Noggin | — | — | 10% | |
| (conditioned medium) | | | | |
| B27 supplement | Gibco | 17504044 | — | |
| N-acetylcysteine | Sigma | A9165 | 1.25 | mM |
| Nicotinamide | Sigma | N0636 | 10 | mM |
| Y-27632 | Tocris | 1254 | 5 | uM |
| A8301 | Tocris | 2939 | 500 | nM |
| SB202190 | Sigma | S7067 | 1 | uM |
| FGF-7 | Peprotech | 100-19 | 5 | ng/ml |
| FGF-10 | Peprotech | 100-26 | 20 | ng/ml |
| Primocin | InvivoGen | ant-pm-1 | 100 | ug/ml |
| Heregulin beta-1 | Peprotech | 100-03 | 5 | nM |
| (only for passage 0) | | | | |

The expansion medium incudes a BMP inhibitor. BMP inhibitor is defined as an agent that binds to a BMP molecule to form a complex wherein the BMP activity is neutralized, for example by preventing or inhibiting the binding of the BMP molecule to a BMP receptor. Alternatively, the inhibitor is an agent that acts as an antagonist or reverse agonist. BMP-binding proteins that can be used in the disclosed methods include, but are not limited to Noggin (Peprotech), Chordin and chordin-like proteins (R&D systems) comprising chordin domains, Follistatin and follistatin-related proteins (R&D systems) comprising a follistatin domain, DAN and DAN-like proteins (R systems) comprising a DAN cysteine-knot domain, sclerostin/SOST (R&D systems), decorin (R&D systems), and alpha-2 macroglobulin (R&D systems). Most preferred BMP inhibitor is Noggin. In some forms, Noggin is present in the basal culture medium in a concentration of about 8% to about 12%. Noggin is preferably added to the basal culture medium at a concentration of at least about 10%.

The expansion medium incudes a WNT agonist. Wnt agonists include the Rspondin family of secreted proteins, which is include of 4 members (R-spondin 1 (NU206, Nuvelo, San Carlos, Calif.), R-spondin 2 ((R&D systems), R-spondin 3, and R-spondin-4); and Norrin. In a preferred embodiment, a Wnt agonist is selected from the group consisting of: R-spondin, Wnt-3a and Wnt-6. Preferred concentrations for the Wnt agonist are about 10% for R-spondin and approximately 100 ng/ml or 100 ng/ml for WNt-3a. In some preferred forms, the WNT agonist is not a GSK inhibitor.

In some forms, the WNT agonist is R Spondin 1 present in an amount from about 8% to about 12%, preferably about 10%. In some forms, R Spondin 1 and/or Noggin are added to the medium as a conditioned medium, which is the medium harvested from stable cell lines expressing R Spondin 1 and Noggin. Percent of an agent in a medium, as used in herein, refers to volume percentage e.g., 10 ml of each conditioned medium can be added to 100 ml basal media to prepare an expansion medium containing 10% of each supplement. Mathematical calculations involved in making solutions are further described in Stephenson, “Calculations for Molecular Biology and Biotechnology”, 3^{rd} Edition, Chapter 2—Solutions, Mixtures, and Media, pages 15-42 (2016), which is incorporated herein in its entirety.

The expansion medium includes a MAPK inhibitor. SB202190 (4-(4-Fluorophenyl)-2-(4-hydroxyphenyl)-5-(4-pyridyl)-1H-imidazole) is a highly selective, potent and cell permeable inhibitor of p38 MAP kinase. SB 202190 binds within the ATP pocket of the active kinase (Kd=38 nM, as measured in recombinant human p38), and selectively inhibits the p38α and β isoforms. Other useful p38 MAPK inhibitors include, but are not limited to SB203580 (4-[5-(4-Fluorophenyl)-2-[4-(methylsulfonyl)phenyl]-1H-imidazol-4-yl]pyridine); SB 203580 hydrochloride (4-[5-(4-Fluorophenyl)-2-[4-(methylsulphonyl)phenyl]-1H-imidazol-4-yl]pyridine hydrochloride); SB202190 (4-[4-(4-Fluorophenyl)-5-(4-pyridinyl)-1H-imidazol-2-yl]phenol); DBM 1285 dihydrochloride (N-Cyclopropyl-4-[4-(4-fluorophenyl)-2-(4-piperidinyl)-5-thiazolyl]-2-pyrimidinamine dihydrochloride); SB 239063 (trans-4-[4-(4-Fluorophenyl)-5-(2-methoxy-4-pyrimidinyl)-1H-imidazol-1-yl]cyclohexanol); SKF 86002 dihydrochloride (6-(4-Fluorophenyl)-2,3-dihydro-5-(4-pyridinyl) imidazo[2,1-b]thiazole dihydrochloride). In some forms, the MAPK inhibitor is SB202190 in a concentration from about 0.5 μM to about 1.5 μM, preferably about 1.0 μM.

The expansion medium includes one or more TGFβ inhibitors. A8301 (3-(6-Methyl-2-pyridinyl)-N-phenyl-4-(4-quinolinyl)-1H-pyrazole-1-carbothioamide) is potent inhibitor of TGF-β type I receptor ALK5 kinase, type I Activin/nodal receptor ALK4 and type I nodal receptor ALK7. A83-01 may be added to the culture medium at a concentration of between 10 nM and 10 μM, or between 20 nM and 5 μM, or between 50 nM and 1 μM. For example, A83-01 may be added to the culture medium at approximately 500 nM. Other useful TGF-type I receptor inhibitors include, but are not limited to SB431542 (4-[4-(1,3-benzodioxol-5-yl)-5-(2-pyridinyl)-1H-imidazol-2-yl]benzamide); LY 364947 (4-[3-(2-Pyridinyl)-1H-pyrazol-4-yl]-quinoline); R 268712 (4-[2-Fluoro-5-[3-(6-methyl-2-pyridinyl)-1H-pyrazol-4-yl]phenyl]-1H-pyrazole-1-ethanol); SB 525334 (6-[2-(1,1-Dimethylethyl)-5-(6-methyl-2-pyridinyl)-1H-imidazol-4-yl]quinoxaline); and SB 505124 (2-[4-(1,3-Benzodioxol-5-yl)-2-(1,1-dimethylethyl)-1H-imidazol-5-yl]-6-methyl-pyridine). In preferred forms, the TGFβ inhibitor is A8301 in a concentration from about 400 nM to about 600 nM, preferably about 500 nM.

The expansion medium includes one or more ROCK inhibitors. Y-27632 (trans-4-[(1R)-1-Aminoethyl]-N-4-pyridinylcyclohexanecarboxamide dihydrochloride) is a selective p160ROCK inhibitor. Other useful Rho inhibitors include isoquinolin and (S)-(+)-2-methyl-1-[(4-methyl-5-isoquinolinyl) sulfonyl]-hexahydro-1H-1,4-diazepine dihydrochloride (H-1152; Tocris Bioscience). In preferred forms, the ROCK inhibitor is Y-27632 in a concentration from about 3 μM to about 7 μM, preferably about 5 μM.

In particularly preferred forms, the expansion or PD cell culture media used in the disclosed methods includes an ErbB3/4 ligand (e.g., human neuregulin β-1). The ErbB receptor tyrosine kinase family consists of four cell surface receptors, ErbB1/EGFR HER1, ii) ErbB2/HER2, iii) ErbB3/HER3, and iv) ErbB4/HER4. ErbB3/4 ligands include members of the neuregulin/heregulin family. The neuregulin/heregulin family is referred to herein as the neuregulin family. The neuregulin family is a family of structurally related polypeptide growth factors that are gene products of alternatively spliced genes NRG1, NRG2, NRG3 and NRG4. In more preferred forms, the excluded one or more ErbB3/4 ligands of the culture medium are polypeptides that are gene products of one or more of NRG1, NRG2, NRG3 and NRG4 i.e., a neuregulin polypeptide).

In some forms, the expansion medium can include one or more supplements, growth factors, and/or antibiotics. Cell culture supplements are growth medium additives that are required for the healthy expansion or differentiation of cells. Certain cell types, such as mammalian cells, require additional compounds in addition to basal media. These include amino acids (such as L-glutamine), glucose, vitamins, and proteins. In some forms, the one or more supplements are selected from the group comprising GlutaMAX® in an amount from about 0.5% to about 1.5%, preferably about 1%, B27 supplement, N-acetylcysteine in concentration from about 1.0 mM to about 1.5 mM, preferably about 1.25 mM, and nicotinamide in concentration from about 7 mM to about 13 mM, preferably about 10 mM.

In some forms, the expansion medium can include one or more growth factors. Growth factors are typically small proteins or peptides that act as signaling molecules, transmitting specific instructions to cells to regulate their behavior. Growth factors are added to the culture medium to provide cells with the necessary signals to support their growth and maintain their specific functions in vitro and facilitate cell proliferation, differentiation, and maintenance of cell viability. In some forms, the one or more growth factors are selected from the group comprising Epidermal growth factor (EGF) and fibroblast growth factor (FGF), wherein the growth factors are preferably FGF-7 in a concentration from about 3 ng/ml to about 7 ng/ml, preferably about 5 ng/ml, and FGF-10 in a concentration from about 15 ng/ml to about 25 ng/ml, preferably about 20 ng/ml.

In some forms, the expansion medium contains one or more antibiotics and/or antimycotics. Antibiotics and antimycotics are often added to cell culture media to help prevent the growth of contaminating bacteria, fungi, and mycoplasma. In addition, certain selection antibiotics are used to select and establish eukaryotic cells modified by genetic engineering. In some forms, the one or more antibiotics are selected from the group comprising Penicillin-Streptomycin in an amount from about 0.5% to about 1.5%, preferably about 1.0%, and Primocin in a concentration from about 75 μg/ml to about 125 μg/ml, preferably about 100 μg/ml.

### 3. Proximal Differentiation Medium

A preferred PD medium is a cell culture medium suitable for culture of nasal epithelial organoids. In some embodiments, the PD medium is serum free and/or BPE (bovine pituitary extract)-free. An example of a suitable PD medium is the commercially available PneumaCult-ALI medium (StemCell Technologies). PneumaCult™-ALI Medium is a serum- and BPE-free medium for the culture of human airway epithelial cells at the air-liquid interface (ALI). Airway epithelial cells cultured in PneumaCult™ ALI Medium undergo extensive mucociliary differentiation to form a pseudostratified epithelium that exhibits morphological and functional characteristics similar to those of the human airway in vivo. PneumaCult™-ALI Medium supports the generation of differentiated nasal organoids in a 2D or 3D culture system.

In a particularly preferred form, the proximal differentiation medium contains basal medium supplemented with a γ-secretase inhibitor in a concentration from about 7 μM to about 13 μM, preferably about 10 μM, and wherein the proximal differentiation medium has a pH of about 7.2 to about 7.6, preferably about 7.4.

In a particularly preferred embodiment, the PD medium is supplemented with a notch inhibitor, preferably in a concentration range between 5 and 30 UM, preferably between 10 and 20 UM and more preferably about 10 μM.

Examples of preferred Notch inhibitors that can be used in the context of this invention are: gamma-secretase inhibitors, such as DAPT or dibenzazepine (DBZ) or benzodiazepine (BZ) or LY-411575, an inhibitor capable of diminishing ligand mediated activation of Notch (for example via a dominant negative ligand of Notch or via a dominant negative Notch or via an antibody capable of at least in part blocking the interacting between a Notch ligand and Notch), or an inhibitor of ADAM proteases. In a particularly preferred embodiment, the notch inhibitor is DAPT ([N—(N-[3,5-difluorophenacetyl]-L-alanyl)-S-phenylglycine t-butyl ester).

The isolated cells cultured in expansion medium are subsequently cultured in PD medium for a period of time effective for formation of PD-organoids. In one preferred form, the time period of time effective for formation of PD-organoids is from about five to about 8 days. In another preferred embodiment, the period of time effective for formation of airway organoids is about 6 to about 7 days.

### 4. 3D Nasal Organoids

An improved procedure for generating the disclosed differentiated 3D nasal organoids has been established. As described in the non-limiting Examples, an array of conditions and differentiation protocols were tested to generate 3D differentiated nasal organoids with similar morphology and function to the mature nasal epithelium.

The method for generating superior differentiated 3D nasal organoids generally includes the following steps:
(i) obtaining nasal turbinate cells capable of differentiating into nasal epithelial cells;
(ii) adhering the nasal turbinate cells from Step (i) onto a support structure that promotes the formation of (3D) organoids;
(iii) culturing the nasal turbinate cells on the support structure from Step (ii) in an expansion culture medium for a period of time to produce 3D nasal undifferentiated cells, wherein the period of time is about 8 days to about 12 days, preferably about 10 days;
(iv) culturing the 3D nasal undifferentiated cells on the support structure from Step (iii) in a basal medium for period of time to enlarge the central lumen, wherein the period of time is about 3 days to about 5 days, preferably about 4 days;
(v) culturing the 3D nasal undifferentiated cells on the support structure from Step (iv) in a proximal differentiation medium for a period of time to generate the 3D differentiated organoid of the human nasal epithelium, wherein the period of time is about 5 days to about 7 days, preferably about 6 days.

In some forms, the expansion culture medium contains basal media. In some forms, the basal media contains one or more buffering agents, one or more growth factors, a ROCK inhibitor, a TGF β inhibitor, a MAPK inhibitor, a WNT agonist, a BMP-4 inhibitor, one or more supplements, and one or more antibiotics.

In some forms, the basal media is Dulbecco's Modified Eagle's Medium (DMEM), Advanced DMEM/F12, RPMI-1640, or Minimum Essential Medium (MEM). In some forms, the basal media is Advanced DMEM-F12. In some forms, the buffering agent is

HEPES buffer. In some forms, the HEPES buffer is present in the basal media in an amount ranging from 0.5% to about 1.5%. Preferably, the HEPES buffer accounts for 1.0% of the total concentration of the basal media. In some forms, the basal medium is Advanced DMEM/F-12 supplemented with about 1% HEPES buffer, about 1% GlutaMAX®, and about 1% Penicillin-Streptomycin.

In some forms, the one or more growth factors are selected from the group comprising Epidermal growth factor (EGF) and fibroblast growth factor (FGF). In a preferred form, the growth factor is FGF-7. In some forms, the FGF-7 is present in a concentration from about 3 ng/ml to about 7 ng/ml. In preferred forms, the FGF-7 is present in about 5 ng/ml. In another embodiment, the FGF is FGF-10. In these forms, the FGF-10 is present in a concentration from about 15 ng/ml to about 25 ng/ml. Preferably the FGF-10 is present in a concentration of about 20 ng/ml.

In some forms, the ROCK inhibitor is Y-27632. In some forms, the ROCK inhibitor is present in a concentration from about 3 μM to about 7 μM. Preferably, the ROCK inhibitor is present in a concentration of about 5 μM. In some forms, the TGF-β inhibitor is A8301. In some forms, the TGF-β inhibitor is in a concentration from about 400 nM to about 600 nM. Preferably, the TGF-β inhibitor is present in a concentration of about 500 nM.

In some forms, the MAPK inhibitor is SB202190 in a concentration from about 0.5 μM to about 1.5 M, preferably 1.0 μM. In some forms, the WNT agonist is R Spondin 1 present in an amount from about 8% to about 12%, preferably about 10%. In some forms, R Spondin 1 and/or Noggin are added to the medium as a conditioned medium, which is the medium harvested from stable cell lines expressing R Spondin 1 and Noggin. Percent of an agent in a medium, as used in herein, refers to volume percentage e.g., 10 ml of each conditioned medium can be added to 100 ml basal media to prepare an expansion medium containing 10% of each supplement. Mathematical calculations involved in making solutions are further described in Stephenson, “Calculations for Molecular Biology and Biotechnology”, 3^{rd} Edition, Chapter 2—Solutions, Mixtures, and Media, pages 15-42 (2016), which is incorporated herein in its entirety. In some forms, the BMP-4 inhibitor is Noggin present in a concentration of about 8% to about 12%, preferably about 10%.

In some forms, the one or more supplements are selected from the group containing GlutaMAX® in an amount from about 0.5% to about 1.5%, preferably about 1%, B27 supplement, N-acetylcysteine in concentration from about 1.0 mM to about 1.5 mM, preferably about 1.25 mM, and nicotinamide in concentration from about 7 mM to about 13 mM, preferably about 10 mM;

In some forms, the one or more antibiotics are selected from the group containing Penicillin-Streptomycin in an amount from about 0.5% to about 1.5%, preferably about 1.0%, and Primocin in a concentration from about 75 μg/ml to about 125 μg/ml, preferably about 100 g/ml.

In some forms, the support structure includes materials selected from the group containing Matrigel®, RGD-functionalized PEG hydrogel crosslinked using factor XIIIa, collagen, hyaluronic acid extracellular matrix, synthetic hydrogels, decellularized extracellular matrix, alginate, Poly (lactic-co-glycolic acid) (PLGA), or electrospun fibers. In a preferred form, the method is performed using a commercially available extracellular matrix. A preferred ECM for use in a method of the invention includes at least two distinct glycoproteins, such as two different types of collagens or a collagen and laminin. In a preferred form, the method is performed with a commercially available extracellular matrix such as MATRIGEL™ (growth Factor Reduced Basement Membrane Matrix), which comprises laminin, entactin, and collagen IV. Other extracellular matrices (ECM) are known in the art for culturing cells. In general, an extracellular matrix comprises laminin, entactin, and collagen. In a preferred form, the method is performed using a 3-dimensional culture device (chamber) that mimics an in vivo environment for the culturing of the cells, where preferably the extracellular matrix is formed inside a plate that is capable of inducing the proliferation of stem cells under hypoxic conditions. Such 3-dimensional devices are known in the art. Other commercially available products include Cultrex® basement membrane extract (BME; Trevigen). Polyethylene glycol (PEG), polyvinyl alcohol (PVA), polylactide-co-glycolide (PLG), and polycaprolactone (PLA) are common materials used to form synthetic scaffolds. Scaffold-free 3D cell spheroids can be generated in suspensions by the forced floating method, the hanging drop method, or agitation-based approaches. Edmondson, et al., Assay Drug. Dev. Technol., 12 (4): 207-218 (2014). For example, the isolated cells are embedding in 60% MATRIGEL™ and seeded in a suspension culture plate prior to culture in the expansion medium.

In some forms, the proximal differentiation medium contains basal medium supplemented with a γ-secretase inhibitor in a concentration from about 7 μM to about 13 μM, preferably about 10 μM, and wherein the proximal differentiation medium has a pH of about 7.2 to about 7.6, preferably about 7.4.

In some forms, the method for generating a 3D organoid further includes replenishing the expansion medium once a day, three times a week, or 4 times a week. In some forms, the method for generating a 3D organoid further includes re-plating and passaging 3D human nasal undifferentiated cells one or more times. In some forms, the method for generating a 3D nasal organoid further includes passaging the undifferentiated nasal cells at a ratio of about 1:3, about 1:4, about 1:5, about 1:6, about 1:7; about 1:9, or about 1:10 or about 1:11.

### 5. 2D Nasal Organoid Monolayers

Methods of generating 2D nasal organoid monolayers are also disclosed. The non-limiting Examples demonstrates an optimized procedure that can be used to generate superior nasal organoid monolayers, which better mimic nasal epithelium exposure to invading pathogens. As described in the Examples, the critical step of the disclosed method for producing optimized 2D nasal organoid monolayers lies in the partial incubation of the undifferentiated nasal cells in a differentiation medium having a slightly acidic pH. This acidic pH has been found to be more representative of the physiological milieu of the surface liquid in the human airways, including the nasal mucosa.

The disclosed method for generating 2D nasal organoid monolayers from the 3D nasal undifferentiated cells include the following steps:
(i) dissociating the 3D nasal undifferentiated cells into single nasal undifferentiated cells;
(ii) transferring the single nasal undifferentiated cells of Step (ii) to a two-chamber support structure and culturing them in expansion medium for a period of time to allow cell attachment and expansion, wherein the period of time is about 1 day to about 3 days; and
(iii) culturing the nasal undifferentiated cells of Step (ii) in a first and second proximal differentiation medium for a total of about 8 days to generate confluent two-dimensional (2D) organoid monolayers.

In some forms, the two-chamber support structure contains a top-chamber and bottom-chamber, and wherein the single nasal undifferentiated cells are adhered to the top chamber of the support structure.

In some forms, Step (iii) further includes: (a) adding the first proximal differentiation medium to the top chamber and the bottom chamber to incubate the nasal undifferentiated cells for about 2 days, wherein the first proximal differentiation medium has a pH of about 7.2 to about 7.6, preferably about 7.4, and (b) replacing the first proximal differentiation medium in the top chamber with the second proximal differentiation medium for about 6 days, wherein the second proximal differentiation medium has a pH of about 6.4 to about 6.8, preferably about 6.6.

In some forms, the dissociated undifferentiated nasal epithelial cells are cultured as a monolayer, preferably on a permeable support (cell culture insert) in expansion medium at 37° C. in a humidified incubator with 5% CO₂ for 1~2 days and then cultured in PD medium as a monolayer for a time period between 5 and 16 days, preferably between 10 and 14 days, and more preferably, for about 12-14 days to obtain 2D organoid monolayers. The PD medium is preferably provided on the apical and basolateral sides of the monolayer. Permeable supports are commercially available, for example, Corning® Transwell®. Transwell inserts are convenient, ready-to-use permeable support devices pre-packaged in standard multiple well plates. The unique, self-centered hanging design prevents medium wicking between the insert and outer well. Transwell inserts are available in a wide variety of sizes, membrane types, and configurations.

### III. Methods of Using Nasal Organoids

Methods of using the in vitro generated organoids and compositions thereof are disclosed. The in vitro generated organoids are particularly suited for research purposes, providing optimized cell models for the study of nasal biology and respiratory disease pathology. The in vitro generated organoids are also particularly suitable for biomarker identification, and evaluation of new molecules and existing therapies for treating respiratory infections and/or diseases. As described above in Section II (A), the in vitro generated organoid can be a 3D nasal organoid or 2D nasal organoid monolayer (hereon referred to as “nasal organoids”).

### A. Providing Cell Models for Studying Biology and Disease Pathology of the Upper Respiratory Tract

The nasal organoids can be used to study respiratory biology in a physiologically relevant manner; thus, enabling improved understanding of the mechanisms underlying function of the nasal epithelial tissue and the impact of pathogens and respiratory diseases. In some forms, the nasal organoids can be used to study the morphological features, cellular interactions, molecular kinetics, and tissue architecture specific to the upper respiratory epithelium. In some forms, the nasal organoids can be used to explore the ciliary beating, mucociliary clearance, mucus production, and barrier function of the nasal epithelium.

The nasal organoids can be used to study the impact of environmental factors and genetic modifications on health and disease. In some forms, the nasal organoids can be used to model various nasal diseases and conditions, including respiratory infections, allergies, chronic rhinosinusitis, and nasal polyps. By incorporating patient-derived cells or genetically modified cells into nasal organoids, researchers can mimic specific disease phenotypes and study the underlying mechanisms of disease development and progression. For example, the nasal organoids can be derived from patient samples or genetically modified to express specific immune factors facilitating the study of individual variations in susceptibility to certain allergens, such as pollen, dust mites, or pet dander. Thus, the nasal organoids can be used to investigate the influence of genetic factors on development of allergic rhinitis (hay fever) and explore potential personalized treatment approaches. Another example is using the nasal organoids to study inflammatory conditions that develop without an immune-mediated allergic response such as those triggered by irritants such as strong odors, smoke, air pollution, temperature changes, or hormonal imbalances.

The nasal organoids provide a platform for studying host-pathogen interactions in the human respiratory tract. For example, researchers can infect nasal organoids with respiratory viruses or bacteria to investigate the cellular responses, immune interactions, and molecular pathways involved in the infection process. This enables a deeper understanding of viral or bacterial pathogenesis and the development of antiviral or antibacterial strategies. In some forms, the nasal organoids can be used to study how pathogens manipulate host cell signaling pathways and immune responses e.g., pathogen evasion strategies, host innate immune responses, and the activation of pro-inflammatory pathways, providing insights into the interplay between pathogens and the nasal epithelium. In some forms, the nasal organoids provide a valuable platform for studying the host immune response to pathogenic infections e.g., activation of immune cells, cytokine production, and the recruitment of immune cells to the site of infection.

In exemplary forms, the nasal organoids can be infected with a wide range of respiratory viruses, including but not limited to influenza, respiratory syncytial virus (RSV), coronaviruses (e.g., SARS-CoV-2), and rhinoviruses. Infection of the organoids with said viruses can facilitate the study of viral entry, replication, and spread within the nasal epithelium, closely mimicking the in vivo infection process. The disclosed nasal organoids also provide a platform to investigate the impact of viral variants and mutations on viral infectivity, replication, and interaction with the nasal epithelium. This knowledge is crucial for understanding the emergence of new viral strains and developing strategies to mitigate their spread. For example, it was demonstrated that the 2D and 3D organoids recapitulated the increased susceptibility of nasal epithelial tissue to infectivity by the SARS-CoV-2 variant strains Delta and Omicron compared to the ancestral wildtype strain (FIGS. 3C-3G).

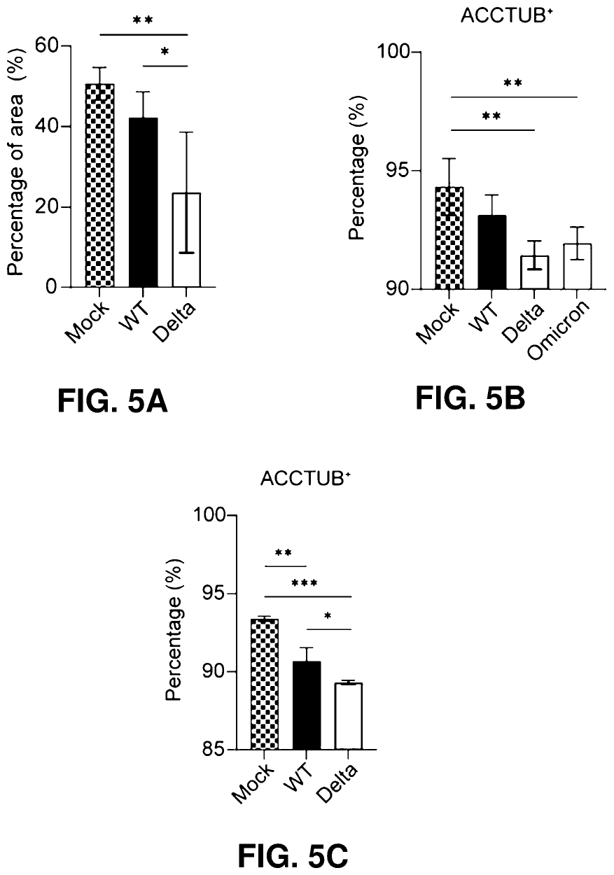

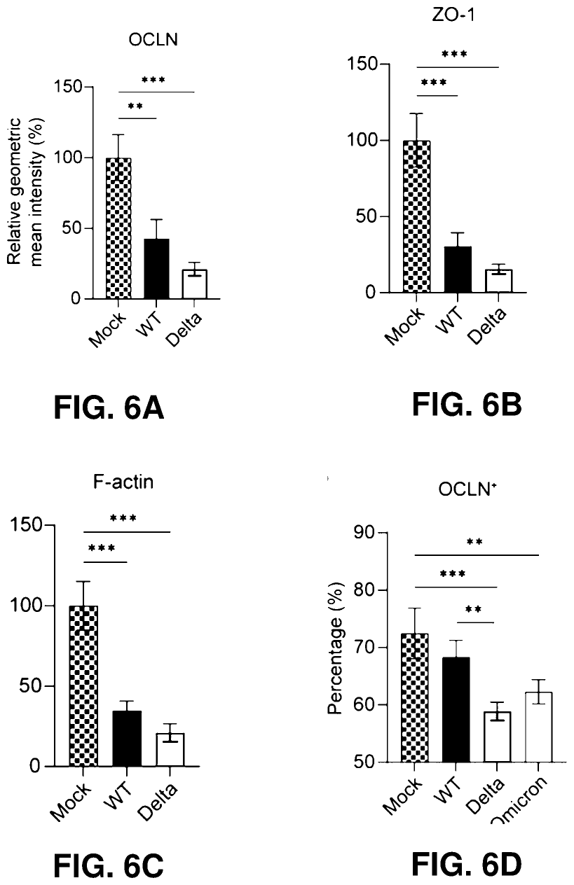

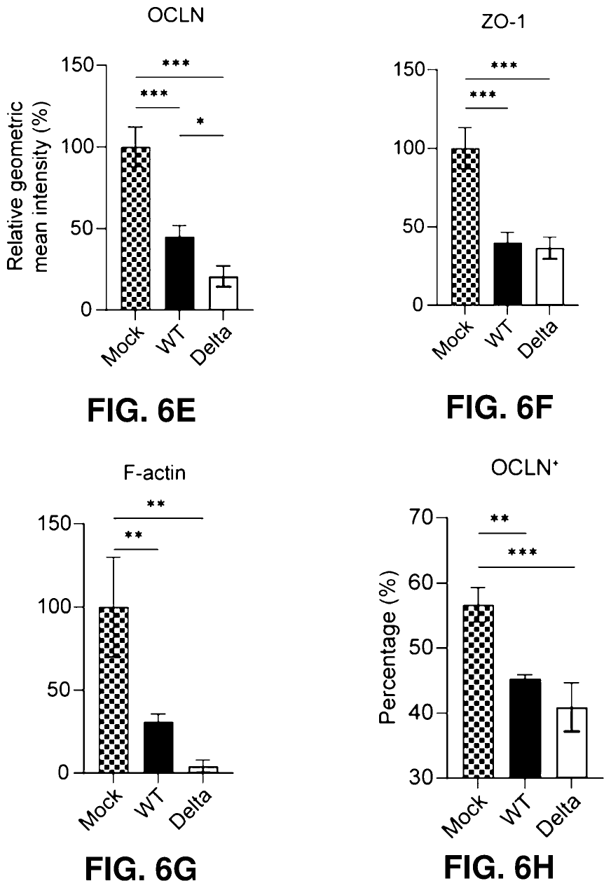

In other exemplary forms, the nasal organoids provide a controlled system to investigate the mechanisms underlying viral pathogenesis by facilitating examination of the interactions between viruses and different cell types within the organoid. Exemplary parameters that can be assessed include but are not limited to cellular tropism, viral replication kinetics, and the impact of viral infection on the nasal tissue microenvironment. The non-limiting Examples demonstrate the use of the 2D and 3D organoids to determine the effects of SARS-CoV-2 infection on cell adhesion such as strength of tight junction connections (FIGS. 6A-6G), and mucociliary clearance by the ciliated and goblet cells (FIGS. 5A-5C).

### 1. Exemplary Pathogens

The nasal organoids can be used for studying a variety of pathogens that infect and/or disrupt the function of the nasal epithelium, including but not limited to viruses, bacteria, and fungi.

a. Coronaviruses

As extensively characterized in the non-limiting Examples, the optimized nasal organoid monolayers adequately recapitulate SARS-CoV-2 high infectivity, particularly the highly transmissible Delta and Omicron variants, in the upper respiratory tract. Notably, in the nasal organoids, the Omicron variant exhibited a significantly higher replicative fitness and infection rate than the earlier circulating variants and outgrew the latter in a competition assay, indicating nasal organoids able to recapitulate the infectivity of emerging SARS-CoV-2 variants (FIGS. 4E-4I). In contrast, the Omicron and Delta variants exhibited lower replicative fitness than the WT strain in Vero E6/TMPRSS2 cells (FIGS. 4M-4P), highlighting the strength of biologically relevant nasal organoids (43).

In some forms, the coronavirus is derived from mammalian or non-mammalian coronaviruses such as for example, human coronaviruses, feline coronaviruses, canine coronaviruses, porcine coronaviruses, bovine coronaviruses, dromedary camel coronaviruses, murine coronaviruses, or variants thereof. In preferred forms, the coronavirus particles are derived from human coronaviruses. In some forms, the coronavirus particles are Human Coronavirus 229E (HCoV-229E) particles, Human Coronavirus OC43 (HCoV-OC43) particles, Human Coronavirus NL63 (HCoV-NL63) particles, Human Coronavirus HKU1 (HCoV-HKU1) particles, Middle East Respiratory Syndrome Coronavirus (MERS-CoV) particles, Severe Acute Respiratory Syndrome Coronavirus 1 (SARS-CoV-1) particles, SARS-CoV-2 particles, and/or variant particles thereof. In some forms, culture coronavirus is an alpha coronavirus, beta coronavirus, delta coronavirus, and/or gamma coronavirus, or sub-variants and/or sub-variant particles thereof.

i. Coronaviruses and SARS-CoV-2

The coronaviruses (order Nidovirales, family Coronaviridae, and genus Coronavirus) are a diverse group of large, enveloped, positive-stranded RNA viruses that cause respiratory and enteric diseases in humans and other animals.

Coronaviruses typically have narrow host specificity and can cause severe disease in many animals, and several viruses, including infectious bronchitis virus, feline infectious peritonitis virus, and transmissible gastroenteritis virus, are significant veterinary pathogens. Human coronaviruses (HCoVs) are found in both group 1 (HCoV-229E) and group 2 (HCoV-OC43) and are historically responsible for ~30% of mild upper respiratory tract illnesses.

At ~30,000 nucleotides, their genome is the largest found in any of the RNA viruses. There are three groups of coronaviruses; groups 1 and 2 contain mammalian viruses, while group 3 contains only avian viruses. Within each group, coronaviruses are classified into distinct species by host range, antigenic relationships, and genomic organization. The genomic organization is typical of coronaviruses, with the characteristic gene order (5′-replicase [rep], spike [S], envelope [E], membrane [M], nucleocapsid [N]-3′) and short untranslated regions at both termini. The SARS-CoV rep gene, which includes approximately two-thirds of the genome, encodes two polyproteins (encoded by ORF1a and ORF1b) that undergo co-translational proteolytic processing. There are four open reading frames (ORFs) downstream of rep that are predicted to encode the structural proteins, S, E, M, and N, which are common to all known coronaviruses.

In some forms, the coronavirus particle is derived from COVID associated with SARS-CoV-2 betacoronavirus of the subgenus Sarbecovirus. SARS-CoV-2 viruses share approximately 79% genome sequence identity with the SARS-CoV virus identified in 2003. The genome organization of SARS-CoV-2 viruses is shared with other betacoronaviruses; six functional open reading frames (ORFs) are arranged in order from 5′ to 3′: replicase (ORF1a/ORF1b), spike(S), envelope (E), membrane (M) and nucleocapsid (N). In addition, seven putative ORFs encoding accessory proteins are interspersed between the structural genes.

In some forms, the coronavirus particles are derived from a variant of SARS-CoV-2, such as SARS-CoV-2 B.1.1.7 (Alpha variant), SARS-CoV-2 B.1.351 (Beta variant), SARS-CoV-2 P.1 (Gamma variant), SARS-CoV-2 B.1.617, SARS-CoV-2 B.1.617.1 (Kappa variant), SARS-CoV-2 B.1.621 (Mu variant), SARS-CoV-2 B.1.617.2 (Delta variant), SARS-CoV-2 B.1.617.3, and SARS-CoV-2 B.1.1.529 (Omicron variant). In some forms, the SARS-CoV-2 particles are SARS-CoV-2 alpha variant particles, SARS-CoV-2 beta variant particles, SARS-CoV-2 gamma variant particles, SARS-CoV-2 delta variant particles, SARS-CoV-2 epsilon variant particles, SARS-CoV-2 eta variant particles, SARS-CoV-2 iota variant particles, SARS-CoV-2 kappa variant particles, SARS-CoV-2 mu variant particles, SARS-CoV-2 omicron variant particles, SARS-CoV-2 zeta variant particles, SARS-CoV-2 1.617.3 variant particles and/or SARS-CoV-2 lambda variant particles.

In some forms, the coronavirus particles can be derived from a sub-variant of the SARS-CoV-2 B.1.1.7 (Alpha variant), a sub-variant of the SARS-CoV-2 B.1.351 (Beta variant), a sub-variant of the SARS-CoV-2 P.1 (Gamma variant), a sub-variant of the SARS-CoV-2 B.1.617, a sub-variant of the SARS-CoV-2 B.1.617.1 (Kappa variant), a sub-variant of the SARS-CoV-2 B.1.621 (Mu variant), a sub-variant of the SARS-CoV-2 B.1.617.2 (Delta variant), a sub-variant of the SARS-CoV-2 B.1.617.3, or a sub-variant of the SARS-CoV-2 B.1.1.529 (Omicron variant), or a sub-variant derived from a descendent lineage of one or more of the foregoing sub-variants. For example, when the SARS-CoV-2 variant is an Omicron variant, the Omicron sub-variant can be a BA.1 sub-variant, a BA.2 sub-variant, a BA.3 sub-variant, a BA.4 sub-variant, a BA.5 sub-variant, or a BA.1/BA.2 circulating recombinant sub-variant such as XE.

ii. SARS-CoV

In some forms, the coronavirus is derived from the replication of SARS-CoV and variants thereof. SARS-CoV predisposes the host to developing severe acute respiratory syndrome, otherwise known as SARS. SARS is caused by the SARS coronavirus, known as SARS CoV. SARS CoV is believed to be a strain of the coronavirus usually only found in small mammals that have mutated, thereby enabling it to infect humans.

A wide range of clinical manifestations are seen in patients with SARS from mild, moderate, to severe and rapidly progressive and fulminant disease. The estimated mean incubation period of SARS-CoV infection was 4.6 days (95% CI, 3.8-5.8 days) and 95% of illness onset occurred within 10 days. The mean time from symptom onset to hospitalization was between 2 and 8 days but was shorter toward the later phase of the epidemic. The mean time from symptom onset to need for invasive mechanical ventilation (IMV) and to death was 11 and 23.7 days, respectively.

The major clinical features of SARS are fever, rigor, chills, myalgia, dry cough, malaise, dyspnea, and headache. Sore throat, sputum production, rhinorrhea, nausea, vomiting, and dizziness are less common. Watery diarrhea was present in 40% to 70% of patients with SARS and tended to occur about 1 week after illness onset. SARS-CoV was detected in the serum and cerebrospinal fluid of 2 patients complicated by status epilepticus. Elderly patients with SARS-CoV infection might present with poor appetite, a decrease in general well-being, fracture as a result of fall, and confusion, but some elderly subjects might not be able to mount a febrile response. In contrast, SARS-CoV infection in children aged less than 12 years was generally mild, whereas infection in teenagers resembled that in adults. There was a low mortality rate among young children and teenagers. SARS-CoV infection acquired during pregnancy carried a case fatality rate of 25% and was associated with a high incidence of spontaneous miscarriage, preterm delivery, and intrauterine growth retardation without perinatal SARS-CoV infection among the newborn infants.

Asymptomatic SARS-CoV infection was uncommon in 2003; a meta-analysis had shown overall sero-prevalence rates of 0.1% (95% CI, 0.02-0.18) for the general population and 0.23% for health care workers (95% CI, 0.02-0.45) in comparison with healthy blood donors, others from the general community, or patients without SARS-CoV infection recruited from the health care setting (0.16%, 95% CI, 0-0.37).

The clinical course of patients with SARS-CoV infection seemed to manifest in different stages. In the first week of illness of SARS-CoV infection, many patients presented with fever, dry cough, myalgia, and malaise that might improve despite the presence of lung consolidation and rising viral loads on serial samples. During the second week, many patients experienced recurrence of fever, worsening consolidation, and respiratory failure, while about 20% of patients progressed to ARDS requiring IMV. Peaking of viral load on day 10 of illness corresponded temporally to peaking of the extent of consolidation radiographically, and a maximal risk of nosocomial transmission, particularly to health care workers.

The SARS-CoV particles can be extracted from patients infected with SARS-CoV and/or variants thereof, for viral studies and/or antiviral drug evaluation.

iii. MERS-CoV

In some forms, the disclosed compositions and methods of using thereof, can be used to investigate and test the Middle East respiratory syndrome-related coronavirus (MERS-CoV) and variants thereof. MERS-CoV predisposes the host to developing Middle East Respiratory Syndrome (MERS). MERS-CoV is a coronavirus believed to be originally from bats. However, humans are typically infected from camels, either during direct contact or indirectly. Spread between humans typically requires close contact with an infected person. As of 2021, there is no specific vaccine or treatment for the disease, although attempts are being made.

The virus MERS-CoV is a member of the beta group of coronaviruses, Betacoronavirus, lineage C. MERS-CoV genomes are phylogenetically classified into two clades, clade A and B. The earliest cases were of clade A clusters, while the majority of more recent cases are of the genetically distinct clade B. MERS-CoV is closely related to the Tylonycteris bat coronavirus HKU4 and Pipistrellus bat coronavirus HKU5.

The specific exposures that lead to sporadic MERS-CoV infections are unknown, therefore it is challenging to estimate the incubation period in primary cases. However, based on data from cases of human-to-human transmission, the incubation period is a median of 5-7 days, with a range of 2-14 days (median 5 2 days [95% CI 1.9-14.7]).

Immunocompromised patients can present with longer incubation periods of up to 20 days.

iv. Common Human Coronaviruses

Unlike the highly pathogenic SARS-CoV, MERS-CoV, and SARS-CoV-2, the four so-called common HCoVs generally cause mild upper-respiratory tract illness and contribute to 15%-30% of cases of common colds in human adults, although severe and life-threatening lower respiratory tract infections can sometimes occur in infants, elderly people, or immunocompromised patients. In some forms, disclosed compositions containing eSTBs and methods of using thereof, can be used to culture the HCoVs for viral studies and to screen for drugs to reduce the replication and ameliorate the pathology associated with one or more of the four common HCoVs.

Human coronavirus 229E (HCoV-229E) is a species of coronavirus which infects humans and bats. HCoV-229E is a member of the genus Alphacoronavirus and subgenus Duvinacovirus. It is an enveloped, positive-sense, single-stranded RNA virus which enters its host cell by binding to the APN receptor. HCoV-229E is associated with a range of respiratory symptoms, ranging from the common cold to high-morbidity outcomes such as pneumonia and bronchiolitis. However, such high morbidity outcomes are almost always seen in cases with co-infection with other respiratory pathogens. In some forms, HCoV-229E may cause acute respiratory distress syndrome (ARDS). HCoV-229E is also among the coronaviruses most frequently co-detected with other respiratory viruses, particularly with human respiratory syncytial virus (HRSV).

Human coronavirus NL63 (HCoV-NL63) is a species of coronavirus, specifically a Setracovirus from among the Alphacoronavirus genus. The virus is an enveloped, positive-sense, single-stranded RNA virus which enters its host cell by binding to ACE2. The virus is found primarily in young children, the elderly, and immunocompromised patients with acute respiratory illness. It also has a seasonal association in temperate climates. The evolution of HCoV-NL63 appears to have involved recombination between an ancestral NL63-like virus circulating in African Triaenops afer bats and a CoV 229E-like virus circulating in Hipposideros bats. Recombinant viruses can arise when two viral genomes are present in the same host cell. The first cases of the infection with HCoV-NL63 were found in young children with severe lower respiratory tract infections admitted to hospitals. While the clinical presentation of the virus can be severe, it has also been found in mild cases of respiratory infection. The comorbidity of HCoV-NL63 with other respiratory infections, has made the specific symptoms of the virus difficult to pinpoint. A study of clinical symptoms in HCoV-NL63 patients without secondary infection, reported the most common symptoms to be fever, cough, rhinitis, sore throat, hoarseness, bronchitis, bronchiolitis, pneumonia, and croup. An early study investigating children with lower respiratory tract illness, found that HCoV-NL63 was more commonly found in outpatients than hospitalized patients, suggesting that it is a common cold virus similar to HCoV-229E and HCoV-OC43, which generally cause less severe symptoms.

Human coronavirus OC43 (HCoV-OC43) is a member of the species Betacoronavirus 1, which infects humans and cattle. The infecting coronavirus is an enveloped, positive-sense, single-stranded RNA virus that enters its host cell by binding to the N-acetyl-9-O-acetylneuraminic acid receptor. Four HCoV-OC43 genotypes (A to D) have been identified, with genotype D most likely arising from genetic recombination. The complete genome sequencing of genotypes C and D and bootscan analysis shows recombination events between genotypes B and C in the generation of genotype D. Of 29 viral variants identified, none belong to the more ancient genotype A. Symptoms of an infection with HCoV-OC43 are as described for HCoV-229E and HCoV-NL63.

Human coronavirus HKU1 (HCoV-HKU1) is an enveloped, positive-sense, single-stranded RNA virus which like the OC43 virus, enters its host cell by binding to the N-acetyl-9-O-acetylneuraminic acid receptor. HCoV-HKU1 has the Hemagglutinin esterase (HE) gene, which distinguishes it as a member of the genus Betacoronavirus and subgenus Embecovirus. Symptoms of an infection with HCoV-HKU1 are as described for HCoV-229E and HCoV-NL63.

v. Non-Human Coronaviruses

The disclosed compositions and methods of using thereof, can be used to study an alpha coronavirus or beta coronavirus that can infect a non-human mammal. In some forms, the alpha coronavirus can be canine enteric coronavirus (CECoV), feline coronavirus (FCoV), porcine respiratory coronavirus (PRCV), porcine epidemic diarrhea virus (PEDV), or transmissible gastroenteritis virus (TGEV). In some forms, the alpha coronavirus can be a variant derived from rhinolophus bat coronavirus HKU2 (Bat-CoV HKU2) or miniopterus bat coronavirus HKU8 (Bat-CoV HKU8). In some forms, the beta coronavirus can be canine respiratory coronavirus (CRCoV), murine coronavirus (M-CoV), porcine hemagglutinating encephalomyelitis virus (PHEV), hedgehog coronavirus 1, bovine coronavirus (B-CoV), or equine coronavirus (E-CoV). In some forms, the beta coronavirus can be a variant derived from tylonycteris bat coronavirus HKU4 (Bat-CoV HKU4), pipistrellus bat coronavirus HKU5 (Bat-CoV HKU5), or rousettus bat coronavirus HKU9 (Bat-CoV HKU9).

The coronavirus may also a gamma coronavirus or a delta coronavirus. In some forms, the gamma coronavirus can be Avian Infectious Bronchitis (AIBV) or Beluga Whale CoV SW1. In some forms, the delta coronavirus can be Bulbul CoV HKU11 (BuCoV HKU11), Thrush CoV HKU12 (ThCoV HKU12), Munia CoV HKU13 (MunCoV HKU13), Porcine CoV HKU15 (PDCoV HKU15), White-eye CoV HKU16 (WECoV HKU16), Sparrow CoV HKU17 (SpCoV HKU17), Magpie Robin CoV HKU18 (MRCoV HKU18), Night heron CoV HKU19 (NHCoV HKU19), wigeon CoV HKU20 (WiCoV HKU20), Common moorhen CoV HKU21 (CMCoV HKU21), falcon CoV HKU27 (FalCoV UAE-HKU27), houbara bustard CoV HKU28 (HouCoV UAE-HKU28), pigeon CoV HKU29 (PiCoV UAE-HKU29), and quail CoV HKU30 (QuaCoV UAE-HKU30), which are the best characterized DCoV species. Delta coronaviruses are described in further detail in Vlasova et al. (2021) Frontiers in Veterinary Science, Vol. 10, doi: 10.3389/fvets.2020.626785. Non-human coronaviruses are described in further detail in Kenney et al. 2020 Veterinary Pathology, Vol. 58, Issue 3, pages 438-452, doi: 10.1177/0300985820980842; Alluwaimi et al. (2020) Frontiers in Veterinary Science, Vol. 7, Article number 582287, doi: 10.3389/fvets.2020.582287).

b. Other Respiratory Viruses

Additional respiratory viruses that can be studied using the nasal organoids include but are not limited to rhinoviruses, influenza viruses, respiratory syncytial virus (RSV), coronaviruses, adenoviruses, parainfluenza viruses, and enteroviruses.

In some forms, the respiratory virus is a rhinovirus. Rhinoviruses are a major cause of the common cold. They primarily infect the upper respiratory tract, including the nasal epithelium, leading to symptoms such as a runny or stuffy nose, sore throat, cough, and sneezing. Thus, in some forms, nasal organoids can be used to model the effects of rhinoviruses on nasal biology.

In some forms, the respiratory virus is an influenza or parainfluenza virus. Influenza viruses, including influenza A and B, are responsible for seasonal flu outbreaks. These viruses infect the respiratory epithelium, causing symptoms such as fever, cough, sore throat, body aches, fatigue, and respiratory distress in severe cases. Parainfluenza viruses are a common cause of respiratory tract infections, especially in young children. They can lead to symptoms such as croup (inflammation of the larynx and trachea), bronchiolitis, and pneumonia. Thus, in some forms, the nasal organoids can be used to model the effects of influenza and parainfluenza viruses on nasal biology.

In some forms, the respiratory virus is Respiratory syncytial virus (RSV). RSV is a common viral pathogen that primarily affects infants and young children. It causes respiratory infections, ranging from mild cold-like symptoms to more severe lower respiratory tract infections, such as bronchiolitis and pneumonia. Thus, in some forms, the nasal organoids can be used to model the effects of RSV on nasal biology, to aid development of more effective vaccines.

In some forms, the respiratory virus is an adenovirus. Adenoviruses can cause respiratory infections, especially in young children. They can infect the upper respiratory tract, leading to symptoms such as a sore throat, cough, runny nose, and fever. Adenoviruses can also cause lower respiratory tract infections, including bronchitis and pneumonia. Thus, in some forms, the nasal organoids can be used to model the effects of adenoviruses on nasal biology, to aid development of more effective vaccines.

In some forms, the respiratory virus is an enterovirus. Some enteroviruses, such as enterovirus D68, can cause respiratory infections, particularly in children. These viruses can lead to symptoms ranging from mild respiratory illness to severe respiratory distress, especially in individuals with underlying respiratory conditions. Thus, in some forms, the nasal organoids can be used to model the effects of enteroviruses on nasal biology, to aid development of more effective vaccines.

c. Bacterial Pathogens

Some respiratory pathogens are able to resist the antimicrobial properties of healthy airway epithelial cells, leading to pathogen persistence and establishment of chronic or acute infection. Exemplary bacteria that can infect the respiratory epithelium include but are not limited to Staphylococcus aureus, Streptococcus pneumoniae, Streptococcus pyogenes, Haemophilus influenzae, Mycobacterium tuberculosis, Mycoplasma pneumoniae, Bordetella pertussis, Legionella pneumophila, Klebsiella pneumoniae, Moraxella catarrhalis, Corynebacterium diphtheriae, and Chlamydia species. These bacteria have diverse infectivity strategies. For example, some bacterial pathogens such as Streptococcus pneumoniae and Haemophilus influenzae, colonize the upper respiratory tract, i.e., the nose or nostrils, nasal cavity, mouth, throat (pharynx), and voice box (larynx). Other bacterial pathogens such as Mycoplasma pneumoniae, infect both the upper and lower respiratory tracts. Some bacterial pathogens are particularly virulent and resistant to multiple antibiotics e.g., Staphylococcus aureus. Thus, the disclosed nasal organoids can be used to model the effects of these bacterial pathogens on disease pathology and nasal biology, to aid development of more effective antibacterial treatments. Bacterial pathogens that infect the respiratory epithelium are further described in Bertuzzi et al., FEMS Microbial Rev., 43 (2): 145-161 (2019); Cappelletty, The Pediatric Infectious Disease Journal, 17 (8): S55-S61 (1998); and Sharma, et al., Front. Immunol., 11:91 (2020); the contents of which are incorporated herein in their entireties.

Exemplary respiratory infections that are caused by one or more of the bacterial species include but are not limited to pneumonia, sinusitis, otitis media (middle ear infection), whooping cough (pertussis), bronchitis, tuberculosis, legionnaires' disease, and diphtheria. Exemplary symptoms of respiratory infections that are caused by one or more of the bacterial species listed above include but are not limited to severe and prolonged coughs, sore throat, and mild to moderate respiratory distress, chest pain, fever, difficulty breathing, and fatigue, muscle aches, swollen lymph nodes, weight loss, facial pain or pressure, nasal and chest congestion, thick nasal discharge, and headache. Thus, the disclosed nasal organoids can be used to study the host immune response to these bacterial infections e.g., activation of immune cells, cytokine production, and the recruitment of immune cells to the site of infection.

d. Fungal Pathogens

Certain fungal species also interact with respiratory epithelial cells and may exploit host cell receptors, leading to a better cell adhesion (and frequent invasion), thereby establishing a fungal infection in the host. Exemplary fungi that can infect the respiratory epithelium include but are not limited to Aspergillus species such as Aspergillus fumigatus, Aspergillus flavus, and Aspergillus niger; Cryptococcus neoformans, Histoplasma capsulatum, Blastomyces dermatitidis, Coccidioides species such as Coccidioides immitis and Coccidioides posadasii. Non-limiting examples of respiratory infections caused by fungal pathogens include but are not limited to allergic bronchopulmonary aspergillosis (ABPA), invasive aspergillosis, cryptococcal meningitis, histoplasmosis, blastomycosis, pneumocystis, and coccidioidomycosis (Valley fever). Fungal pathogens and their resulting respiratory infections are further described in Barros et al., J. Fungi (Basel), 8 (6): 548 (2022); Crossen et al., J. Fungi (Basel), 9 (1): 40 (2022); and Li et al., Front. Immunol., 10:1524 (2019), the contents of which are herein incorporated in their entireties. Thus, the disclosed nasal organoids can be used to study fungal infection dynamics and the host immune response to these fungal infections.

### 2. Non-Pathogenic Respiratory Conditions and Diseases

The nasal organoids also provide a more physiologically relevant system that can be used for studying mechanisms of non-pathogenic respiratory conditions and diseases. These conditions and diseases are typically non-infectious and may arise due to various factors, including environmental triggers, genetic predisposition, or underlying physiological abnormalities. Exemplary non-pathogenic respiratory conditions and diseases include but are not limited to allergic rhinitis, asthma, Chronic Obstructive Pulmonary Disease (COPD), occupational lung diseases, idiopathic pulmonary fibrosis (IPF), pulmonary fibrosis, bronchiectasis, cystic fibrosis, and interstitial lung diseases.

In some forms, the respiratory condition or disease is allergic rhinitis or asthma. Allergic rhinitis, commonly known as hay fever, is an inflammatory condition of the nasal passages triggered by allergens such as pollen, dust mites, pet dander, or mold spores. It leads to symptoms such as nasal congestion, sneezing, itching, and a runny nose. Allergic rhinitis is caused by an immune system response to these allergens rather than a pathogenic infection. Asthma is a chronic respiratory condition characterized by inflammation and narrowing of the airways. It can be triggered by various factors, including allergens, irritants (such as smoke or strong odors), exercise, or respiratory infections. Asthma leads to recurrent episodes of wheezing, coughing, shortness of breath, and chest tightness. While respiratory infections can trigger asthma symptoms, the condition itself is not caused by pathogens. Thus, the nasal organoids can be engineered to contain disease-relevant cell types, such as mucus-secreting goblet cells in allergic rhinitis or airway smooth muscle cells in asthma.

In some forms, the respiratory condition or disease is cystic fibrosis and/or interstitial lung disease. Cystic Fibrosis is a genetic disorder that affects the production and function of mucus, leading to the accumulation of thick and sticky mucus in the lungs and other organs. It results in chronic lung infections, persistent coughing, and difficulty breathing. Interstitial Lung Disease is a group of lung disorders characterized by inflammation and scarring of the lung tissue. It can lead to progressive breathing difficulties, cough, and reduced lung function. Thus, in some forms, the nasal organoids can be generated using patient-derived cells with cystic fibrosis transmembrane conductance regulator (CFTR) gene mutations to mimic the dysfunctional CFTR seen in cystic fibrosis, thereby allowing the study of the effects of CFTR dysfunction on the respiratory epithelium and investigate disease mechanisms.

In some forms, the respiratory condition or disease is Chronic Obstructive Pulmonary Disease (COPD). COPD is a progressive lung disease primarily caused by long-term exposure to irritants, particularly cigarette smoke. It encompasses conditions such as chronic bronchitis and emphysema, characterized by persistent cough, excessive mucus production, shortness of breath, and reduced airflow in and out of the lungs. COPD is primarily a non-infectious disease resulting from chronic exposure to irritants. Thus, in some forms, the nasal organoids can be exposed to cigarette smoke extract or other COPD-relevant irritants to induce disease-like phenotypes. This exposure can replicate the chronic inflammation, oxidative stress, and tissue damage observed in COPD. Researchers can study how the respiratory epithelium responds to these irritants and investigate the underlying mechanisms contributing to COPD pathogenesis.

In some forms, the respiratory condition or disease is categorized as an occupation lung disease. Certain occupational exposures can lead to respiratory diseases. For example, occupational asthma can develop due to exposure to workplace allergens or irritants like dust, chemicals, or fumes. Occupational lung diseases, such as silicosis (caused by inhalation of crystalline silica particles) or asbestosis (resulting from exposure to asbestos fibers), are non-infectious conditions affecting the respiratory epithelium. Thus, the nasal organoids can be exposed to specific occupational toxins or irritants commonly encountered in certain workplaces, such as silica dust, asbestos fibers, coal dust, or various chemicals. By exposing organoids to these substances, researchers can simulate the effects of occupational exposure on the respiratory epithelium and investigate the resulting cellular and molecular changes.

In some forms, the respiratory condition or disease is Idiopathic Pulmonary Fibrosis (IPF). IPF is a progressive and irreversible lung disease characterized by the formation of scar tissue in the lungs. The exact cause of IPF is unknown, and it is believed to result from a combination of genetic and environmental factors. IPF leads to symptoms such as persistent dry cough, shortness of breath, fatigue, and gradual loss of lung function. Thus, in some forms, the nasal organoids can be derived from individuals with familial forms of IPF or specific genetic variants associated with the disease can be generated, allowing researchers to study how these genetic factors influence disease development, progression, and response to potential therapies. These patient-specific nasal organoids contribute to personalized medicine approaches and the identification of genotype-specific therapeutic targets.

### B. Drug Screening and Evaluation

The compositions are particularly suitable for screening and identifying potential candidate agents for the treatment of respiratory infections and diseases. An exemplary method of screening and evaluating candidate agents for treating respiratory infections and/or diseases include one or more of the following steps:
(i) contacting the in vitro generated organoid with a pre-determined concentration of an infectious or disease agent and incubating the in vitro generated organoid for a pre-determined period of time;
(ii) measuring one or more parameters indicative of a pathological cellular phenotype associated with the infection or disease of interest;
(iii) administering a pre-determined concentration of a candidate agent to the in vitro generated organoid incubating the in vitro generated organoid for a pre-determined period of time;
(iv) measuring one or more parameters indicative of cellular response to the candidate agent; and
(v) assessing the efficacy of the candidate agent by comparing the measured parameters of step (iv) to measured parameters of an untreated in vitro generated organoid, wherein a significant difference between the measured parameters of step (iv) and the measured parameters of the untreated control indicates that the candidate agent can be considered for treating a respiratory infection or disease.

In some forms, parameters indicative of a cellular response to the test compound includes but are not limited to the effect of the agent on survival, proliferation, differentiation, morphologic parameters, genetic parameters, or functional parameters of the in vitro generated organoid.

In some forms, the candidate agent may be nucleic acids or analogs thereof, polypeptides or analogs thereof, antibodies, chemicals, small molecules, and/or any combination thereof. In some forms, the treated and untreated cells from the 2D and 3D nasal organoids are evaluated using PCR techniques, immunoassays, sequencing, biochemical assays, functional assays, cell viability assays, microscopy, or combinations thereof. Methods for screening agents are known in the art and include but are not limited to scintillation proximity assays, Direct fluorescence measurement, Fluorescence polarization, Fluorescence resonance energy transfer (FRET), Time-resolved fluorescence (TRF, HTRF, and TiRF), AlphaScreen, High-content screening (HCS), Protein fragment complementation assays (PCA), microfluidics, flow cytometry, and label-free technologies (reviewed in Janzen (2014) Chemistry and Biology Vol 21 (9), pages 1162-1170).

In some forms, the predetermined concentration and predetermined period of time in steps (i) and (iii), respectively, are optimized based on the type of candidate agent. In some forms, the candidate agent is selected from a library of compounds comprising natural products, synthetic compounds, or a combination thereof.

In some forms, the candidate agent is an antiviral agent, antibacterial agent, antifungal agent, or anti-allergenic agent, wherein the effect of the agent is indicative of the agent being safe for the treatment of respiratory infections and/or diseases.

### Evaluation/Screening of Candidate Agents

Methods of using the nasal organoids for evaluating and screening candidate agents are disclosed. The method steps typically include contacting the in vitro generated organoid with the candidate agent, and determining the effect of the agent on survival, proliferation, differentiation, morphologic parameters, genetic parameters, or functional parameters of the in vitro generated organoid.

In some forms, the nasal organoids are cultured under conditions suitable to induce differentiation of the desired cell population as disclosed herein and one or more candidate agents can be applied to the cultured differentiated 2D and 3D nasal organoids, and evaluated for the ability to treat one or more symptoms of the diseased, dysfunctional, or defective cells. The symptom or symptoms can be specific to the disease state being studied or can be of a general nature. Physiological, phenotypic, morphological, or molecular symptoms and other markers of the cells can be monitored over time.

### 1. Exemplary Antimicrobial Agents

In some forms, the nasal organoids can be used for high-throughput drug screening to identify potential antimicrobial compounds. In some forms, the nasal organoids can be used to evaluate the efficacy of antiviral drugs, assess their impact on viral replication, and examine their effects on host immune responses. In this way, the organoids can aid in the discovery and development of new therapeutic strategies to combat microbial infections. For example, in some forms, the disclosed organoids can be used to screen drug safety, toxicity, efficacy and to evaluate test compounds for drug therapies.

In some forms, the disclosed nasal organoids can be used as a platform for determining the effect of candidate compounds on the inhibition of the microbe prior to evaluating of the efficacy of the candidate agent in clinical trials or the approval of the candidate agent for further drug testing to determine drug safety.

In some forms, the disclosed cells can be used to determine the drug efficacy for existing drugs e.g., FDA approved drugs, or the compound is newly discovered, whether it is effective in clinical usage might be evaluated using nasal organoids according to clinical symptom or other testing for therapies.

In an exemplary form, compounds are tested for toxicity and/or the ability to further improve one or more wildtype functions. For example, in some forms, the nasal organoids can be generated from the established differentiation protocol and adopted as a screening platform to identify one or more candidate agents that are compatible with the development of inactivated vaccines.

In an exemplary form, the disclosed nasal organoids, and compositions thereof, are useful for investigating the activity or applicability of one or more candidate agents to treat or alleviate one or more symptoms of a viral infection and/or a disease or disorder associated with a viral infection. In these forms, the candidate agent can be added at different stages of viral infection e.g., pre-infection (before introducing the virus) or post-infection (before introducing the virus). In some forms, test compounds might also be pre-incubated with the virus (neutralization). In these forms, various tests can help reveal how an antiviral works (e.g., does it neutralize the virus or does it stop viral entry). For example, given the lack of effective treatments available for the treatment of coronaviruses, the disclosed compositions and methods are particularly suitable for investigating the activity or efficacy of candidate agents to treat or alleviate or prevent one or more symptoms of any of the coronaviruses described below.

In exemplary forms, the disclosed nasal organoids may be used to screen an agent for an effect on coronavirus infected cells, the method including contacting the coronavirus-infected nasal organoid with the candidate agent and determining the effect of the agent on survival, proliferation, differentiation, or morphologic, genetic, or functional parameters of the various cell types of the nasal organoids. Therefore, in some forms, the methods include one or more steps for assessing the replication of a coronavirus in the presence of one or more active agents.

In some forms, the agent could be an existing FDA-approved drug, with known indications. In some forms, the agent is an unknown compound that has not been reported for its antiviral effect. In some forms, the agent could be one or more chemically synthesized compounds. In some forms, the agent could be one or more components extracted from natural plants or herbs.

In some forms, the candidate agent is an antibacterial therapeutic agent. An exemplary antibacterial agent is Mupirocin (Bactroban Nasal™). In some forms, the candidate agent is an antifungal therapeutic agent e.g., Amphotericin B (Amphocin™) and Nystatin (Mycostatin™).

In some forms, the candidate agent is an antiviral therapeutic agent. In some forms, the antiviral therapeutic agent is an antiviral drug, monoclonal antibody, corticosteroid, immunomodulatory drug, cell therapy, or anticoagulant. Non-limiting examples of antiviral drugs include but are not limited to Remdesivir (GS-5734), Molnupiravir, Favipiravir, and Ivermectin. Remdesivir (GS-5734), an inhibitor of the viral RNA-dependent, RNA polymerase with in vitro inhibitory activity against SARS-CoV-1 and the Middle East respiratory syndrome (MERS-CoV), was identified early as a promising therapeutic candidate for COVID-19 because of its ability to inhibit SARS-CoV-2 in vitro. Molnupiravir (LAGEVRIO™) is an oral antiviral drug that interferes with viral replication. Favipiravir (T-705) is an antiviral drug that inhibits viral RNA polymerase. Non-limiting examples of monoclonal antibodies include REGEN-COV® (casirivimab and imdevimab, administered together), bamlanivimab and etesevimab, administered together, and Sotrovimab (VIR-7831); the last of which targets the spike protein of SARS-CoV-2. An exemplary corticosteroid is dexamethasone, a corticosteroid with anti-inflammatory effects, primarily used for severe COVID-19 cases or those requiring respiratory support. Non-limiting examples of immunomodulatory drugs include tocilizumab and baricitinib. Tocilizumab (Actemra™) is an immunosuppressive drug that blocks the interleukin-6 (IL-6) receptor, used to manage severe inflammatory responses in COVID-19 patients. Baricitinib (Olumian™) is an immunomodulatory drug that inhibits the Janus kinase (JAK) pathway, used in combination with remdesivir for hospitalized patients. An exemplary antiviral cell therapy is convalescent plasma collected from individuals who have recovered from COVID-19 and contains antibodies against the virus. Non-limiting examples of anticoagulants include heparin and enoxaprin, used to prevent or treat blood clots, which may occur in severe COVID-19 cases due to the hypercoagulable state associated with the disease.

In some forms, the candidate agent is a vaccine such as an antiviral vaccine. In some forms, the vaccine is a viral vector-based vaccine, an mRNA vaccine, a protein subunit vaccine, or an inactivated viral vaccine. Exemplary mRNA vaccines include Pfizer-BioNTech (Comirnaty™), an mRNA vaccine requiring two doses administered 3 to 4 weeks apart; and Moderna, an mRNA vaccine requiring two doses administered 4 weeks apart. Exemplary viral vector-based vaccines include Oxford-AstraZeneca (Vaxzevria™, Covishield™), a viral vector-based vaccine requiring two doses administered 4 to 12 weeks apart; Johnson & Johnson (Janssen™), a single dose viral vector-based vaccine; and CanSinoBIO™, a single dose viral vector-based vaccine. Exemplary inactivated viral vaccines include Sinovac, an inactivated virus vaccine which requires two doses administered 2 to 4 weeks apart; Sinopharm, an inactivated virus vaccine which requires two doses administered 2 to 4 weeks apart; and Bharat Biotech (Covaxin™); this inactivated virus vaccine requires two doses administered 4 to 6 weeks apart. Exemplary protein subunit vaccines include Covovax (Novavax™), a protein subunit vaccine requiring two doses administered 3 weeks apart; EpiVacCorona, a protein subunit vaccine requiring two doses administered 3 to 4 weeks apart; Abdala, a protein subunit vaccine requiring three doses administered 2 weeks apart; Soberana 02, a protein subunit vaccine requiring two doses administered 28 days apart; Covovax (Biovac™), a protein subunit vaccine under development; and Covovax, a protein subunit vaccine that is under development.

In some forms, the antiviral vaccine is an influenza vaccine e.g., Trivalent Inactivated Influenza Vaccine (TIV); Quadrivalent Inactivated Influenza Vaccine (QIV); Live Attenuated Influenza Vaccine (LAIV); and Recombinant Influenza Vaccine. In some forms, the antiviral vaccine is a Respiratory Syncytial Virus (RSV) vaccine e.g., Palivizumab (Synagis™), a monoclonal antibody given to prevent severe RSV infection in high-risk infants.

In some forms, the candidate agent is a bacterial vaccine such as a pertussis vaccine e.g., Diphtheria, Tetanus, and Acellular Pertussis (DTaP) Vaccine, and Tetanus, Diphtheria, and Acellular Pertussis (Tdap) Vaccine. In some forms, the bacterial vaccine is a tuberculosis vaccine e.g., Bacillus Calmette-Guérin (BCG) vaccine.

Accordingly, in some forms, the methods involve the step of contacting the nasal organoids with one or more of antiviral drugs such as remdesivir and PAXLOVID™, and monoclonal antibodies such as casirivimab, imdevimab, bamlanivimab, etesevimab, baricitinib, and sotrovimab, and determining the effect of the agent on survival, proliferation, differentiation, or morphologic, genetic, or functional parameters of the nasal organoids based on the disclosed methods. In some forms, the treated and untreated coronavirus-infected nasal organoids are evaluated using PCR techniques, immunoassays, sequencing, biochemical assays, functional assays, cell viability assays, microscopy, or combinations thereof.

### 2. Exemplary Non-Microbial Respiratory Agents

In some forms, the nasal organoids can be used for high-throughput drug screening to identify potential compounds for the treatment of non-pathogenic respiratory conditions. In some forms, the candidate agent is a nasal steroid such as Fluticasone propionate (Flonase™), Mometasone furoate (Nasonex™), Budesonide (Rhinocort™), Triamcinolone acetonide (Nasacort™), and Beclomethasone dipropionate (Qnasl™).

In some forms, the candidate agent is an antihistamine e.g., Loratadine (Claritin™), Cetirizine (Zyrtec™), and Fexofenadine (Allegra™). In some forms, the candidate agent is a decongestant e.g., Pseudoephedrine (Sudafed™), Phenylephrine (Sudafed PE™), and Oxymetazoline (Afrin™). In some forms, the candidate agent is an anticholinergic e.g., Ipratropium bromide (Atrovent™) and tiotropium (Spiriva™). In some forms, the candidate agent is a mast cell stabilizer e.g., Cromolyn sodium (NasalCrom™).

### C. Biomarker Identification

Understanding of the molecular mechanisms driving the pathology of conditions and diseases of the respiratory epithelium is scarce due to a combination of factors including lack of a model that more accurately represents the nasal tissue microenvironment. The disclosed nasal organoids and/or cells/tissues therefrom are ideal for recapitulating respiratory disease phenotypes; thus, providing a more accurate model for biomarker discovery, and identification of molecular targets for the treatment of respiratory diseases.

Methods of using animal models for the identification of biomarkers and potential molecular targets are known in the art. An exemplary method for identifying biomarkers of pathogenic and non-pathogenic respiratory diseases and conditions, includes one or more of the following steps:
(i) contacting the in vitro generated organoid with a pre-determined concentration of an infectious or disease agent and incubating the in vitro generated organoid for a pre-determined period of time;
(ii) isolating and purifying nucleic acids, proteins, and/or metabolites from the cells of the in-vitro generated organoid;
(iii) measuring expression level of the nucleic acids, proteins, and/or metabolites;
(iv) comparing the expression levels of the nucleic acids, proteins, and/or metabolites to corresponding expression levels in the nucleic acids, proteins, and/or metabolites of control in vitro generated organoid-derived cells; and
(v) measuring one or more parameters indicative of cellular response to the candidate agent; and
(vi) identifying one or more biomarkers of the respiratory disease or condition based on statistically significant differences in expression levels of the nucleic acids, proteins, and/or metabolites between the diseased cells and the control cells.

Methods of extracting and analyzing nucleic acids, proteins, or metabolites from tumor tissue are known in the art. Suitable methods include but are not limited to PCR, qPCR, RNA-seq, mass spectrometry, ELISA, Western blot, immunohistochemistry, and metabolic profiling., and any combination thereof. Unless otherwise specified, the practice of the methods described herein can take advantage of the techniques of cell biology, cell culture, molecular biology, transgenic biology, microbiology, recombinant DNA, and immunology. These techniques are explained in detail in the following literature, for example: Molecular Cloning A Laboratory Manual, 2nd Ed., ed. By Sambrook, Fritsch and Maniatis (Cold Spring Harbor Laboratory Press: 1989); DNA Cloning, Volumes I and II (D. N. Glovered, 1985); Oligonucleotide Synthesis (M. J. Gaited., 1984); Mullis et al U.S. Pat. No. 4,683,195; Nucleic Acid Hybridization (B. D. Hames& S. J. Higginseds. 1984); Transcription and Translation (B. D. Hames& S. J. Higginseds. 1984); Culture Of Animal Cell (R. I. Freshney, Alan R. Liss, Inc., 1987); Immobilized Cells And Enzymes (IRL Press, 1986); B. Perbal, A Practical Guide To Molecular Cloning (1984), the series, Methods In Enzymology (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Caloseds., 1987, Cold Spring Harbor Laboratory); Immunochemical Methods In Cell And Molecular Biology (Mayer and Walker, eds., Academic Press, London, 1987); Hand book Of Experimental Immunology, Volumes V (D. M. Weir and C. C. Blackwell, eds., 1986); and Manipulating the Mouse Embryo, (Cold Spring Harbor Laboratory Press, Cold Spring Harbor, N.Y., 1986), each of which is incorporated herein in its entirety by reference.

In some forms, the methods of biomarker identification include profiling the genome of the infected or diseased nasal organoid cells. An exemplary genome analysis includes performing segmentation analysis of the genome profile, identifying loci, and prioritizing the identified loci; and relating to respiratory disease or respiratory infection querying genes at the identified loci. In some forms, gene queries at identified loci are based on gene expression data. In some forms, gene queries at the identified locus are based on an in vitro screening assay.

The identified biomarker is generally a biomarker associated with a respiratory infection or disease. The identified biomarker can be a diagnostic biomarker, a prognostic biomarker, or a pharmacogenomic biomarker. In some forms, identified biomarker is a nucleic acid biomarker or a protein biomarker.

### EXAMPLES

### Example 1

### Materials and Methods

### Establishment, Expansion and Differentiation of Nasal Organoids.

The study was approved by the Institutional Review Board of the University of Hong Kong/Hospital Authority Hong Kong West Cluster (UW21-695). Ten lines of human nasal organoids (NsO) were established using the freshly isolated nasal epithelial cells from nine healthy donors. Briefly, nasal swabs were collected from the nasal turbinate regions using the specimen collection flocked swab (iClean, Biomed Diagnostics). After washing and digestion with 10× TrypLE Select (Gibco) for 5 minutes at 37° C., the resultant single cells were embedded in 70% Matrigel and seeded into a 24-well suspension culture plate. After solidification, droplets were incubated in the expansion medium (Table 1) at 37° C. in a humidified incubator with 5% CO₂. The expansion medium was replenished every other day; the 3D undifferentiated organoids were passaged every 1 to 2 weeks with a ratio between 1:3 to 1:10, depending on whether mechanical shearing or enzymatic digestion was used to split the organoids (Li C, et al. 2022. JoVE, doi: 10.3791/63684: e63684).

To induce the maturation of nasal organoids into 3D differentiated nasal organoids (3D dNsO), the 3D nasal organoids was first incubated in a basal medium (Advanced DMEM/F-12 (Gibco) supplemented with 1% HEPES, 1% GlutaMAX and 1% Penicillin/Streptomycin) for 4 days, followed by incubation with the PD medium (PneumaCult-ALI medium (STEMCELL Technologies) supplemented with 10 μM DAPT) for 6-10 days. The medium was replenished every other day. To induce differentiation into differentiated nasal organoid monolayers (dNsO-mono), 3D nasal organoids were treated with 10× TrypLE Select (Invitrogen) for 5 minutes at 37° C. and seeded the single cells onto Transwell inserts at a density of approximately 4×10⁵ cells per cm². The cells were cultured in the expansion medium for 1 to 2 days, then changed to the PD medium and incubated for another 8-12 days. The PD medium was supplied to both the top and bottom chambers and was replenished every other day. To optimize organoid monolayer differentiation, after the cells reached complete confluency in the PD medium in 1 to 2 days, the media in the top chambers was changed to the PD medium buffered with PIPES (Sigma Aldrich) to create a pH around 6.6, while those in the bottom chambers remained buffered with HEPES (Gibco) to maintain a pH around 7.4. Organoids were incubated in these buffered PD media for 6-10 days to achieve maturation. A Millicell ERS-2 Volt-Ohm Meter (EMD Millipore) was used to measure the trans-epithelial electrical resistance (TEER) of organoid monolayers every other day to assess the epithelial barrier integrity.

The 3D undifferentiated nasal organoids, 3D differentiated nasal organoids, and nasal organoid monolayers were harvested and applied to RNA extraction using MiniBEST Universal RNA Extraction kit (Takara), followed by reverse transcription using Transcriptor First Strand cDNA Synthesis Kit (Roche) and oligo (dT) primer. The resultant cDNAs were used to measure mRNA expression levels of cellular genes (Table 2) using the LightCycler 480 SYBR Green I Master Mix (Roche) (Zhou J, et al. 2019. EMBO Mol Med, Vol. 11, doi:10.15252/emmm.201809528). Photomicrographs and videos of the organoids were acquired using Nikon Eclipse TS100 Inverted Routine Microscope.

TABLE 2
List of qPCR Primers

| | Sequence | |
|---|---|---|
| Gene | (F = Forward; R = Reverse) | |
| SARS-CoV-2 | F | CGCATACAGTCTTRCAGGCT (SEQ ID NO: 1) |
| RdRp | R | GTGTGATGTTGAWATGACATGGTC (SEQ ID NO: 2) |
| | Probe | FAM-TTAAGATGTGGTGCTTGCATACGTAGAC- |
| | | IABkFQ (SEQ ID NO: 3) |
| | R | GATGGCCCCAATCACAGGAA (SEQ ID NO: 4) |
| P63 | F | CAGACTCAATTTAGTGAGCC (SEQ ID NO: 5) |
| | R | CTGCTGGTCCATGCTGTT (SEQ ID NO: 6) |
| CK5 | F | GAGGAATGCAGACTCAGTGGA (SEQ ID NO: 7) |
| | R | TAGCTTCCACTGCTACCTCCG (SEQ ID NO: 8) |
| FOXJ1 | F | TCGTATGCCACGCTCATCTG (SEQ ID NO: 9) |
| | R | CGGATTGAATTCTGCCAGGT (SEQ ID NO: 10) |
| SNTN | F | GCTGCAAACCCAATTTAGGA (SEQ ID NO: 11) |
| | R | TGCTCATCAAGTTCAGAAAGGA (SEQ ID NO: 12) |
| MUC5AC | F | CCTACAAAGCTGAGGCCTGT (SEQ ID NO: 13) |
| | R | GACCCTCCTCTCAATGGTGC (SEQ ID NO: 14) |
| CC10 | F | AGCATCATTAAGCTCATGGAAAAA (SEQ ID NO: 15) |
| | R | GTGGACTCAAAGCATGGCAG (SEQ ID NO: 16) |
| ACE2 | F | CATTGGAGCAAGTGTTGGATCTT (SEQ ID NO: 17) |
| | R | GAGCTAATGCATGCCATTCTCA (SEQ ID NO: 18) |
| FOLR1 | F | CTGGAACTGGACTTCAGGGTT (SEQ ID NO: 19) |
| | R | CACTCCCTCGGCTGTAGTTG (SEQ ID NO: 20) |
| LMO3 | F | GGCTCTTTGGTGTAACGGGA (SEQ ID NO: 21) |
| | R | TGTCTCCAACACAAAATCTCTGA (SEQ ID NO: 22) |
| IRX2 | F | CTACGAGCCCAAGAAAGATGCC (SEQ ID NO: 23) |
| | R | GCCCACTTACTTGCATTGCTG (SEQ ID NO: 24) |
| FOXG1 | F | GAGGTGCAATGTGGGGAGAA (SEQ ID NO: 25) |
| | R | TTCTCAAGGTCTGCGTCCAC (SEQ ID NO: 26) |
| SIX3 | F | CACTACCAGGAGGCCGAGAA (SEQ ID NO: 27) |
| | R | TTGGGGTAGGGGTCCTGTAG (SEQ ID NO: 28) |
| PAX7 | F | GTGAGTTCGATTAGCCGCGT (SEQ ID NO: 29) |
| | R | GTCCAGCCGGTTCCCTTTG (SEQ ID NO: 30) |
| GAPDH | F | GGAGCGAGATCCCTCCAAAAT (SEQ ID NO: 31) |
| | R | GGCTGTTGTCATACTTCTCATGG (SEQ ID NO: 32) |

### Virus Infection and Detection.

SARS-CoV-2 isolate HKU-001a (WT, GenBank accession number MT230904), Delta variant (B.1.617.2; GenBank OM212471) and Omicron variant (B.1.1.529; GenBank OM212473) were propagated in Vero E6/TMPRSS2 cells (JCRB1819) purchased from JCRB cell bank and titrated with plaque assay (Zhao X, et al. 2021, Stem Cell Reports Vol. 16, pages 493-504, doi:10.1016/j.stemcr.2021.02.009; Zhou J, et al. 2020. Nat Med Vol. 26, pages 1077-1083, doi:10.1038/s41591-020-0912-6). Differentiated organoids were always used for infection experiments. 3D nasal organoids were sheared mechanically using a glass Pasteur pipette and incubated with the virus for 2 hours at 37° C. The inoculated organoids were re-embedded into Matrigel and then incubated in the basal medium.

After washing twice with the basal medium, nasal organoid monolayers were apically incubated with the virus for 2 hours at 37° C., followed by incubation in the basal medium. To assess replication kinetics, after inoculation in organoid monolayers with an MOI of 0.1 or inoculation in 3D organoids with an MOI of 1, cell-free culture media were harvested at the indicated hours post-infection, followed by RNA extraction using the MiniBEST Viral RNA/DNA Extraction Kit (Takara) and detection of viral loads (viral gene copy number of RdRp gene) by one-step RT-qPCR assay, and viral titration by TCID₅₀ assay (Zhao X, et al. 2021, Stem Cell Reports Vol. 16, pages 493-504, doi:10.1016/j.stemcr.2021.02.009; Zhou J, et al. 2020. Nat Med Vol. 26, pages 1077-1083, doi:10.1038/s41591-020-0912-6). All experiments with live viruses were conducted in biosafety level 3 laboratories.

### Virus Competition Assay

To compare the replication fitness of different SARS-CoV-2 strains, the nasal organoid monolayers were infected with a 1:1 mixture of WT and the Omicron variant, or a 1:1 mixture of Delta and Omicron variants at a total MOI of 0.1, or the plaque-purified each strain of the virus at an MOI of 0.1. At the indicated hours post-infection, cell-free culture media was harvested, followed by RNA extraction using the QIAamp Viral RNA Mini Kit (Qiagen) and viral gene detection using a one-step probe-based RT-qPCR assay with a primer pair TGGACCTTGAAGGAAAACAGGG (SEQ ID NO:33) and TGGTTCTAAAGCCGAAAAACCC (SEQ ID NO:34) (Peacock T P, et al. 2022, bioRxiv, doi:10.1101/2021.12.31.474653:2021.12.31.474653; Lamers M M, et al. 2022, bioRxiv, doi:10.1101/2022.01.19.476898:2022.01.19.476898). The specific probe for WT and Delta variant was CTATTAATTTAGTGCGTGATCT-HEX (SEQ ID NO:35); the specific probe for the Omicron variant is TTATAGTGCGTGAGCCAGAAGA-FAM (SEQ ID NO:36). A LightCycler 96 system was used to simultaneously detect the FAM and HEX dye in each sample using the endpoint genotyping protocol template. The endpoint fluorescence (EPF) value of each dye in each sample was analyzed using the endpoint genotyping method with the LC96 software. EPF values of the samples from organoids infected with the purified virus (WT-only, Delta-only, or Omicron-only) were used for normalization. The percentage of each variant is calculated as the FAM/HEX value divided by the sum of FAM and HEX for each sample.

### Immunofluorescence Staining and Flow Cytometry.

The cellular composition of the nasal organoids was characterized using specific antibodies (Table 3) to recognize ciliated (ACCTUB), goblet (MUC5AC), club (CC10) and basal cells (P63, CK5), followed by secondary antibodies. Cellular proteins including ACE2, TMPRSS2, OCLN, and ZO-1 in the organoids were labeled with corresponding antibodies. To identify virus-infected cells, the virus-inoculated or mock-infected organoids were stained using an in-house-made antibody against SARS-CoV-2 nucleoprotein (NP) raised in a guinea pig (Zhao X, et al. 2021, Stem Cell Reports Vol. 16, pages 493-504, doi:10.1016/j.stemcr.2021.02.009; Zhou J, et al. 2020. Nat Med Vol. 26, pages 1077-1083, doi:10.1038/s41591-020-0912-), after fixation with 4% PFA for 1 hour at room temperature, permeabilization with 0.5% Triton X-100 for 10 minutes and blocking with protein block (Dako) for 1 hour. To define cellular tropism and to assess cilia abundance, the infected organoids were co-stained with the α-NP and α-ACCTUB. Z-stack was performed to scan nasal organoid monolayers, followed by orthogonal projection to generate the cross-sectional images. To examine cellular junctions, the infected organoids were co-stained with the α-NP and α-OCLN or α-ZO-1. Nuclei and actin filaments were counterstained with DAPI (Thermo Fisher Scientific) and Phalloidin-647 (Sigma-Aldrich), respectively. The organoids were whole mounted on a glass slide with ProLong™ Glass Antifade Mountant (Invitrogen) after staining. Confocal images were acquired using a Carl Zeiss LSM 800 confocal microscope. For image quantification of cilia abundance, the area covered with cilia was manually defined and measured using the ImageJ software. Five randomly selected frames were analyzed for each group. For image quantification of cellular junction proteins, the geometric mean fluorescent intensity of each channel was measured using the Histo function of the ZEN blue software. Logarithmic binning was used; the lower and upper threshold were set to 1 and 65534, respectively.

For flow cytometry analysis, organoids were treated with 10 mM EDTA (Invitrogen) for 30-60 minutes at 37° C. and dissociated into single cells, followed by 5 fixation with 4% PFA for 15 minutes at room temperature, permeabilization with 0.1% Triton X-100 for 5 minutes at 4° C., and then immunostaining using the specific antibodies (Table 3) and the corresponding isotype controls for gating. To determine infection rate, after an MOI of 1 inoculation, organoids were dissociated into single cells, fixed with 4% PFA, followed by immunostaining using an α-double-stranded RNA antibody (dsRNA, 10010500, Scicons); mock-infected organoids were used for gating. A BD FACSCantoII Analyzer or LSR Fortessa was used for analysis. FlowJo software was used for data processing.

TABLE 3
List of Antibodies

| | | Catalog | Lot | Dilution | Dilution |
|---|---|---|---|---|---|
| Name | Supplier | Number | Number | (IF) | (Flow) |
| Mouse IgG1 | ABCAM | ab91353 | GR3327311-3 | — | 1:100 |
| Rabbit IgG | ABCAM | ab172730 | GR3284310-8 | — | 1:100 |
| Goat IgG | R&D systems | AB-108-C | ES4120071 | — | 1:100 |
| Rat IgG | Invitrogen | 10700 | RA226537 | — | 1:100 |
| ACE2 | R&D systems | AF933 | HOK0620051 | 1:100 | 1:100 |
| TMPRSS2 | Invitrogen | PA5-14264 | SI2433683G | — | 1:100 |
| P63 | ABCAM | ab124762 | GR303296-1 | 1:100 | — |
| CK5 | ABCAM | ab128190 | GR239416-18 | — | 1:100 |
| TUBULIN | ABCAM | ab179509 | GR252919-6 | | 1:1000 |
| TUBULIN | Sigma-Aldrich | T7941 | 088M4793 | 1:100 | 1:1000 |
| CC10 | R&D systems | MAB4218 | YWN0218091 | | 1:100 |
| MUC5AC | ABCAM | ab3649 | GR126783 | | 1:1000 |
| SARS-CoV-2 NP | in-house | — | — | 1:1000 | — |
| J2 (dsRNA) | Scicons | 10010500 | 17741 | — | 1:100 |
| OCLN | Santa Cruz | sc-133256 | F0517 | 1:50 | |
| | Biotech | | | | |
| OCLN | ABCAM | ab216327 | GR3243495-20 | | 1:60 |
| ZO-1 | Santa Cruz | sc-33725 | D1317 | 1:50 | |
| | Biotech | | | | |
| ZO-1 | ABCAM | ab221547 | GR3354270-15 | 1:100 | |
| Goat anti-Mouse | Invitrogen | A-11001 | 2090562 | 1:500 | 1:500 |
| 488 | | | | | |
| Goat anti-Mouse | Invitrogen | A-11005 | 1750828 | 1:500 | — |
| 594 | | | | | |
| Goat anti-Rabbit | Invitrogen | A-11034 | 1885241 | 1:500 | 1:500 |
| 488 | | | | | |
| Goat anti-Rabbit | Invitrogen | A-11037 | 1608397 | 1:500 | — |
| 594 | | | | | |
| Donkey anti- | Invitrogen | A-11055 | 1771339 | 1:500 | 1:500 |
| Goat 488 | | | | | |
| Donkey anti-Rat | Invitrogen | A-21208 | 1900239 | — | 1:500 |
| 488 | | | | | |
| Goat anti- | Invitrogen | A-11073 | 1990462 | 1:500 | — |
| Guinea Pig 488 | | | | | |

### Scanning Electron Microscopy

After infection of the indicated viruses with an MOI of 5 or mock infection, nasal organoid monolayers were mounted and coated after sequential fixation with 2.5% glutaraldehyde and 1% osmium. Sample processing was performed by Electron Microscope Unit of the University of Hong Kong. A LEO 1530 FEG Scanning Electron Microscope was used for image acquisition.

### Statistical Analysis.

Statistical analysis was conducted using GraphPad Prism 9.0. Student's t-test or ANOVA test were used to determine statistical significance as specified in the Brief Description of the Drawings. Data in the figures represent technical replicates, i.e., the mean and s.d. of a representative experiment. The number of technical replicates is indicated in figure legends. The experiments were independently performed two to four times in multiple organoid lines, as indicated in the figure legends. * p≤0.05, ** p≤0.01, *** p≤0.001.

### Results

### Derivation, Differentiation, and Characterization of Nasal Organoids

Nasal epithelial cells were collected from the inferior turbinate of healthy donors using a flocked swab. The nasal epithelial cells were embedded in Matrigel and then overlaid with the organoid expansion medium supplemented with niche factors including R-spondin, Noggin, FGF7, and FGF10, the same medium for deriving human lung organoids (FIG. 1A). Cystic organoids emerged on day 2 or day 3 and became enlarged gradually. The nasal organoids were passaged every 10-12 days over 6 months. More than 10 lines of nasal organoids from different donors were established with a 100% success rate, indicating the robustness and reproducibility of the protocol. Distinct from the lung organoids that always have a central lumen throughout expansion culture, nasal organoids normally develop a small central lumen in late days during each passage; most nasal organoids are solid cellular spheres devoid of a discernible central lumen.

The derived nasal organoids were propagated in the expansion medium in a 3-dimensional (3D) format by maintaining the clonogenic potential of adult stem cells or progenitor cells and directing the nasal organoids towards an immature state. To simulate the mature nasal epithelium, an array of conditions and defined differentiation protocols were tested to generate differentiated 3D organoids and organoid monolayers (FIG. 1A). Briefly, after 3D undifferentiated nasal organoids grew in the expansion medium for 10 days, differentiation was initiated with a 4-day incubation with a basal medium to enlarge the central lumen. This is an indispensable step since solid organoids without a central lumen may not develop adequate mucociliary differentiation; motile cilia were barely discernible in these solid organoids. Subsequently, the basal medium was replaced with a proximal differentiation (PD) medium, the same differentiation medium for airway organoid maturation (18), and further incubated for 6 days to generate fully differentiated nasal organoids. Hitherto, dense, and motile cilia were readily discernible in every differentiated nasal organoid (data not shown). The organoids growing three-dimensionally in Matrigel normally develop an apical-in polarity since the extracellular matrix, herein provided by Matrigel, controls the epithelial polarity (24, 31).

Compared to the undifferentiated nasal organoids, the 3D differentiated nasal organoids exhibited a significant upregulation of cell type markers for basal cell (P63, CK5) and ciliated cell (FOXJ1, SNTN), as well as ACE2, the SARS-CoV-2 cellular receptor (FIG. 1B). The differentiated nasal organoids accommodated all airway epithelial cell types, i.e., ciliated, basal, goblet and club cells (data not shown). An apical-out nasal organoid was deliberately displayed to highlight the dense ACCTUB+ cilia. The expression of compartment-specific genes were also examined in 3D differentiated nasal organoids compared to that in 3D differentiated airway organoids derived from three different donors. Nose-enriched and trachea/bronchi-enriched genes were significantly higher in differentiated nasal and airway organoids, respectively (FIGS. 1C-1H), indicating that nasal organoids derived from nasal cells indeed retained the compartment-specific gene expression profile, consistent with the observations in native tracheobronchial and nasal epithelial cells as revealed by single-cell sequencing studies (6, 7).

The airway epithelium lining the human respiratory tract forms a physical barrier to protect against invading microbes, which is particularly important for the nasopharyngeal epithelium since it represents the very frontline to the external environment. To better model nasal epithelium exposure to invading pathogens, differentiated nasal organoids of 2D format were generated (also referred to as organoid monolayers). Briefly, single cells dissociated from 3D undifferentiated nasal organoids were seeded onto transwell inserts and incubated in the expansion medium for 2 days, which allowed cell attachment and expansion. They were then switched to the PD medium and incubated for 8 days to generate confluent monolayers of differentiated nasal organoids. The 3D differentiated nasal organoids, basal cell (P63, CK5), ciliated cell (FOXJ1, SNTN) markers, and the ACE2 receptor were significantly upregulated in differentiated nasal organoid monolayers (FIG. 1I). Four airway epithelial cell types are present in the organoid monolayers (data not shown). Ciliated cells, the major cell population in the human nasal epithelium, are notably enriched in the organoid monolayers. The beating cilia were readily discernible under a microscope (data not shown). Trans-epithelial electrical resistance (TEER) is a reliable indication of epithelial integrity, the essential characteristic of a physical barrier. TEER was measured during the 10-day differentiation culture. TEER increased and reached a plateau on day 6 (data not shown). Thus, at the end of differentiation culture on day 10, the mature nasal organoid monolayers form an intact epithelial barrier on transwell inserts (FIG. 1J). Collectively, expandable nasal organoids were established with a high success rate from readily accessible nasal epithelial cells. The expansion medium sustained the long-term expansion of nasal organoids by directing the organoids toward an immature state. The differentiation protocol enables the generation of differentiated nasal organoids of 3D and 2D format that faithfully simulate the nasal epithelium. These 3D differentiated nasal organoids and organoid monolayers of 2D format remain stable for around two weeks and one month respectively, applicable to various experimental manipulations.

### Optimization of Differentiated Nasal Organoid Monolayers

The surface liquid in the human airways, including nasal mucosa, is slightly acidic with an average pH of 6.6 (32). Moreover, the low pH of airway surface liquid is one of the determinants for SARS-CoV-2 active infection (33, 34). However, in the differentiation culture of organoid monolayers as described above, the proximal differentiation (PD) media in both top and bottom chambers were buffered with HEPES at a pH of 7.4, the physiological pH of interstitial fluid. It was hypothesized that a slightly acidic medium in the top chamber and a physiological alkaline medium in the bottom chamber might better simulate the in vivo milieu and generate more physiological-relevant nasal organoids. To this end, after the cells reached confluency on transwell inserts, the HEPES buffer was replaced in the top medium (i.e., the PD medium) with a PIPES buffer to create a pH of 6.6, while the original HEPES remained in the bottom medium with a pH of 7.4. Namely, organoid monolayers were incubated in the modified differentiation medium with a pH of 6.6/7.4 (top/bottom) or the original differentiation medium with a pH of 7.4/7.4 (FIG. 2A). The nasal organoids incubated in the differentiation medium at pH 6.6/7.4 displayed a higher TEER than those at pH 7.4/7.4, indicating the formation of a strengthened epithelial barrier (FIG. 2B).

We then examined the cellular composition and ACE2 and TMPRSS2 in all the differentiated nasal organoids, including 3D differentiated organoids and organoid monolayers undergoing differentiation at pH 6.6/7.4 or pH 7.4/7.4, in comparison with those in the 3D undifferentiated nasal organoids. It was found that ciliated cells represented the major cell population in nasal organoid monolayers (FIGS. 2G-2J). Goblet cells were significantly enriched in nasal organoid monolayers at pH 6.6/7.4 than those at pH 7.4/7.4 and 3D differentiated nasal organoids (FIGS. 2K-2N). Interestingly, the proportion of basal cells was the highest in 3D differentiated nasal organoids (FIGS. 2C-2F). In contrast to ciliated cells, club cells significantly decreased after differentiation culture. Of note, the differentiation procedure increased ACE2+ cells in all variants of organoids; the percentage of ACE2+ cells was the highest in nasal organoid monolayers at pH 6.6/7.4. In addition, TMPRSS2+ cells were significantly more abundant in organoid monolayers than both differentiated and undifferentiated 3D organoids (FIGS. 3A-3J). Collectively, among all the differentiated organoids, nasal organoid monolayers differentiated in pH 6.6/7.4 most adequately phenocopy the native nasal epithelium for modeling SARS-CoV-2 infection.

### SARS-CoV-2 Replication and Higher Replicative Fitness of Emerging Variants in Nasal Organoids

SARS-CoV-2 replication kinetics in differentiated nasal organoids derived from four different donors was examined. Both differentiated 3D nasal organoids and organoid monolayers sustained SARS-CoV-2 replication, as shown by increased viral RdRp gene copy number and infectious titer (FIGS. 3A-3D), although organoids derived from different donors showed variable replication capacity. The replicative fitness of SARS-CoV-2 was higher in organoid monolayers than in the 3D counterparts, which might be ascribed to the higher ACE2 and TMPRSS2 expression in the former than the latter, as shown in FIGS. 3A-3J. Immunostaining showed many SARS-CoV-2-infected cells in nasal organoid monolayers; viral NP co-localizing or not co-localizing with ACCTUB+ ciliated cells in both en face and cross-sectional images (data not shown).

The Delta and Omicron variants overtook the prior circulating strains after the emergence. To elucidate virus-host interaction underlying their enhanced transmissibility over the ancestral viruses, an ancestral wildtype (WT) strain, a Delta strain and an Omicron strain were inoculated onto the nasal organoid monolayers induced differentiation at pH 6.6/7.4 since these differentiated nasal organoids best model SARS-CoV-2 infection. Quantification of viral load in the culture medium and viral titration revealed the higher replicative fitness of the Omicron variant than the Delta variant, followed by the WT strain (FIGS. 4E and 4F). The Omicron variant showed a significantly higher viral titer of around 2 log units than the WT strain. The infection rate was examined, i.e., the percentage of virus-infected cells 24 hours after a high MOI inoculation. Flow cytometry analysis demonstrated significantly higher infectivity of the Omicron variant than the Delta variant; the lowest infection rate was found in the WT strain (FIG. 4G).

A pairwise competition assay in the nasal organoid monolayers. The Omicron variant quickly outgrew the Delta variant (FIG. 4H). The replicative advantage of the Omicron variant was even more dramatic when it was co-inoculated with the WT strain (FIG. 41). The replicative fitness of three virus strains in differentiated airway organoid monolayers were compared. The airway organoids reproduced a similar order of replicative capacity of these three virus strains, yet the Omicron's replicative advantage was less remarkable than that in the nasal organoid monolayers (FIGS. 4J and 4K). The higher replication capacity of the Omicron variant than the WT virus was verified in the airway organoids by the competition assay. The outgrowth of the Omicron variant was not as prominent as that in nasal organoids (FIG. 4L). In contrast, replication kinetics and competition assays in Vero E6/TMPRSS2 cells revealed a lower replicative fitness of Delta and Omicron variants than the WT strain (FIGS. 4M-4P), which is contradictory to the higher infectivity and transmissibility of these variants observed in humans.

Overall, the three virus strains exhibited differential replicative fitness and infectivity in differentiated nasal organoid monolayers, which accurately reproduced the Omicron's highest transmissibility as observed in real-life epidemiological observations. In addition, nasal organoids recapitulated the variable transmissibility more adequately than airway organoids.

### Preferential Infection of Ciliated Cells by SARS-CoV-2

The infected nasal organoid monolayers were examined by confocal imaging to elucidate the cellular pathology of SARS-CoV-2 infection. Ciliated cells are distributed evenly in the mock-infected nasal organoid monolayers, with dense ACCTUB+ cilia on the apex of ciliated cells (data not shown). At 24 hours post-inoculation (1 MOI), WT-, or Delta-infected or mock-infected dNsO-mono were co-stained with α-NP (red) and α-ACCTUB (green stain). Nuclei and actin filaments were counterstained with DAPI (blue) and Phalloidin-647 (white). The remaining cilia on an infected ciliated cell were examined. Ciliary damage and loss of ciliated cells in SARS-CoV-2 infected nasal organoids were observed, consistent with the observations reported in cultured human airway epithelial cells (11). The dense hairy cilia on the apex of normal ciliated cells were disrupted and/or deformed into a fibrous clump (data not shown). In the organoids infected by the WT virus, two small clusters of cilia remained on a ciliated cell, whereas most cilia were destroyed. Ciliary damage was more prominent in organoids infected by the Delta variant than those infected by WT strain. Image analysis in 5 randomly selected frames revealed a significantly decreased proportion of ACCTUB+ cells in the virus-infected organoids, especially those infected with the Delta variant (FIG. 5A). Flow cytometry analysis verified the results of image analysis. While WT virus infection modestly depleted ACCTUB+ ciliated cells in nasal organoid monolayers, the infection of the Delta variant or the Omicron resulted in a further and significant depletion (FIG. 5B). Consistently, flow cytometry analysis of the airway organoid monolayers infected by the WT and Delta variant showed similar results (FIG. 5C). Scanning electron microscopy was conducted to examine the cytopathological changes in more detail. In the uninfected nasal organoids, we visualized dense cilia with a smooth surface on the apex of ciliated cells (data not shown). At 24 hours post-inoculation (1 MOI), mock-, WT-, or Delta-infected dNsO-mono were applied to scanning electron microscopy. However, cilia became shorter, tangled, and deformed in the organoids infected by the WT strain and the Delta variant (data not shown). Thus, SARS-CoV-2 preferentially infected ciliated cells in nasal organoids and airway organoids, causing ciliary damage and loss.

### SARS-CoV-2 Infection Disrupting Cell Adhesion to Enhance Virus Transmission

Apart from the mucociliary clearance afforded by ciliated and goblet cells, the formation of tight junctions between adjacent epithelial cells represents a subcellular level of host defense to protect against invading pathogens and maintain the homeostasis of the human respiratory tract. Meanwhile, viruses have developed multiple mechanisms to disassemble tight junctions to facilitate infection or exploit specific cell junction targets for cellular entry (35). At 24 hours post-inoculation (1 MOI), mock-, WT-, or Delta-infected dNsO-mono were co-stained with (A) α-NP (green) and α-OCLN (red), or (B) α-NP (green) and α-ZO-1 (red). Nuclei and actin filaments were counterstained with DAPI (blue) and Phalloidin-647 (white). Zonula Occludens (ZO) family link tight junction proteins such as Occludin and Claudins to the actin cytoskeleton. Occludin (OCLN) and ZO-1 proteins delineate the outline of each cell in mock-infected nasal organoid monolayers, indicating the formation of intact tight junctions by these adhesion molecules. Altered cell junctions in the organoids infected with the WT and Delta viruses were observed. In the organoids infected by WT strain, ZO-1 and OCLN diffused or diminished in the area within or near the virus-infected cells, whereas Delta variant infection caused a widespread disassemble of cellular junction throughout the monolayers, not limited to the infected cells, suggesting a possible paracrine effect. The signal intensity in 3 randomly selected frames were quantified and found a significantly eliminated OCLN and ZO-1 in WT virus-infected nasal organoids compared to mock-infected organoids. The organoids inoculated with the Delta variant exhibited a further diminished ZO-1 (FIG. 6B), OCLN (FIG. 6A) and cytoskeleton F-actin (FIG. 6C) than those infected by the WT virus. Flow cytometry analysis verified SARS-CoV-2 induced disruption of OCLN, and a more disruptive effect of the Delta and Omicron variants in nasal organoid monolayers (FIG. 6D). The destructive effect of SARS-CoV-2 infection on cellular junction was further verified in airway organoid monolayers by immunostaining (FIGS. 6E-6G) and flow cytometry analysis (FIG. 6H). Thus, SARS-CoV-2 disrupted tight junctions in nasal organoids and airway organoids, breaching the epithelial barrier.

### Discussion

The mucosal epithelium lining the nasopharyngeal compartment is the entry portal and primary target of respiratory pathogens, including SARS-CoV-2; meanwhile nasal epithelial cells express the highest level of SARS-CoV-2 entry factors among multiple interrogated human tissues (36). High susceptibility of nasal epithelial cells to SARS-CoV-2, robust viral replication in these cells and subsequent viral shedding constitute the biological basis underlying viral pathogenesis and its high transmissibility. As such, it is imperative to establish an in vitro model of the nasal epithelium to elucidate SARS-CoV-2 infection and virus-host interaction. In this study, a robust organoid culture system of the human nasal epithelium was established and characterized to model SARS-CoV-2 upper respiratory infection. It is a two-phase culture system, i.e., expansion and differentiation culture (FIG. 1A). The expansion medium sustained long-term expansion of nasal organoids for more than 6 months by directing the 3D undifferentiated organoids toward an immature state. The differentiation protocols were defined to generate differentiated 3D nasal organoids and organoid monolayers (FIGS. 1B and 1I). The differentiated nasal organoids adequately simulate the native nasal epithelium. Namely, the nasal organoid culture system enables a highly efficient reconstruction and stable expansion of the human nasal epithelium in culture plates for various experimental applications.

The differentiation condition was optimized, and highly differentiated nasal organoid monolayers were generated by mimicking the slightly acidic surface fluid on the nasal mucosa (FIG. 2A). Among all the differentiated forms of nasal organoids, organoid monolayers induced differentiation with the slightly acidic medium (pH 6.6) in the top chamber displayed a more intensified epithelial barrier (FIG. 2B) and a higher expression of ACE2, the cellular receptor for SARS-CoV-2 infection (FIGS. 3A-3J). Notably, the optimized nasal organoid monolayers accurately reproduce the higher infectivity and elevated replicative fitness of the Omicron variant than prior circulating variants by flow cytometry analysis of infection rate, viral titration detection of viral propagation and the competition assay (FIGS. 4E-41).

A significant advance is the cell source for deriving nasal organoids. Nasal epithelial cells were procured from the inferior turbinate of healthy donors using a flocked swab, which is a non-invasive procedure, unlike deriving lung organoids using cells from bronchoalveolar lavage or resected tissues that require the invasive manipulations of physicians and surgeons. More importantly, prior studies of human intestinal organoids, the first adult stem cell-derived organoid model, revealed that the epithelial organoids generated from different intestinal segments retain the structural and functional characteristics from which they are derived (29), since tissue identity is imprinted in the adult stem cells and maintained in these organoids during long-term expansion culture (37). Thus, nasal epithelial cells would be an optimal source for deriving physiologically relevant nasal organoids. Indeed, nasal organoids exhibited a compartment-specific gene expression profile (FIGS. 3A-3J). More importantly, nasal organoids retain the genotypic and phenotypic characteristics of original cells, which allows the generation of personalized nasal organoids and provides a facile and robust in vitro model of upper respiratory epithelium for various applications of personalized medicine.

Interestingly, nasal organoids have been derived with a great success rate, which was beyond expectations. This superior efficiency was justified in a prior study in which nasal cells from healthy individuals and COVID-19 patients were harvested via nasal brushing. Among all the harvested cells, about 60% are ciliated and goblet cells, whereas suprabasal and club cells accounted for approximately 30% of the cells (5). Suprabasal cells are an intermediate cell population between basal and club cells (6). Basal/suprabasal and club cells were reported to be progenitor cells for respiratory epithelial regeneration (38-40). These progenitor cells have clonal expansion capacity in vitro and proliferate to form organoids (40-42). In this study, the expansion medium supplemented with R-spondin, Noggin and FGF sustains the clonogenicity of these progenitor cells and enables a consecutive expansion of undifferentiated nasal organoids for approximately 6 months. The facile cell procurement procedure circumvented the relatively shorter expandability of nasal organoids, highlighting nasal organoids as a robust and personalized in vitro model of the nasal epithelium. Notably, in the nasal organoids, the Omicron variant exhibited a significantly higher replicative fitness and infection rate than the earlier circulating variants and outgrew the latter in a competition assay, indicating nasal organoids able to recapitulate the infectivity of emerging SARS-CoV-2 variants (FIGS. 4E-41). In contrast, the Omicron and Delta variants exhibited lower replicative fitness than the WT strain in Vero E6/TMPRSS2 cells (FIGS. 4M-4P), highlighting the strength of biologically relevant nasal organoids (43).

It appears that SARS-CoV-2 targeted both ciliated cells and non-ciliated cells. The subsequent interrogations imply that those infected “non-ciliated” cells, at least part of them, result from ciliary damage and/or depletion mediated by the virus infection (FIGS. 5A-5C). SARS-CoV-2 cellular tropism in ciliated cells, the resultant ciliary damage and depletion of ciliated cells were documented in SARS-CoV-2 infection in cultured primary nasal and airway epithelial cells and single-cell RNA sequencing studies of COVID-19 patients (4, 5, 10, 11, 13, 14). Ciliated cells are the primary target of SARS-CoV-2. Accordingly, the higher replicative fitness of the Omicron and Delta variants in nasal organoid monolayers led to a more prominent depletion of the ciliated cell (FIG. 5B). Motile cilium-driven mucociliary clearance represents an important host defense against pathogens. Ciliary damage caused by SARS-CoV-2 would inevitably compromise the mucociliary clearance, leading to exaggerated tissue pathology. Cellular adhesion, including tight junction, represents an additional layer of host defense to protect against invading pathogens. It was demonstrated that the more infective Omicron and Delta variants disrupted tight junctions more severely than the earlier variant through imaging analysis and quantitative flow cytometry analysis (FIGS. 6A-6H). Collectively, SARS-CoV-2 viruses, especially the more transmissible variants, destroy ciliated cells leading to a compromised mucociliary clearance, and disassemble tight junctions to breach the epithelial barrier. These pathological changes render the uninfected cells more vulnerable, thereby facilitating virus spread and transmission.

Altogether, a robust nasal organoid culture system and fully characterized the organoids were established and characterized. The nasal organoids are stably passaged for more than 6 months, providing a renewable source of nasal epithelial cells. Although not intended to be limiting, the following are exemplary features of the nasal organoids generated in the current study: (1) the nasal organoids were derived from a nasal epithelial cells obtained via a nasal swab, (2) the organoids have long-term expandability i.e., they can be passaged continuously up to six months or more, (3) the organoids produce multiple cell types including ciliated cells, basal cells, goblet cells, and club cells, (4) the organoids were extensively characterized via light microscopy for brightfield images and video of beating cilia, RT-qPCR for cell-type specific markers and compartment-specific markers, IF staining for cell-type identification and formation of tight junction, scanning electron microscopy for ultrastructural images of cilia, TEER for barrier integrity, flow cytometry for cell-type composition and expression of host dependency factor, (5) the organoid types that were produced include 3D dNSO, 2D dNSO (at pH 7.4), and 2D optimized dNsO (pH 6.6/7.4); and (6) replication kinetics of the organoids were extensively characterized via RT-qPCR and TCID₅₀-~3-4 log 10 unit for ancestral viruses and ~5-6 log 10 unit for Delta/Omicron variants. The 2D and 3D nasal organoid monolayers were optimized by mimicking the native microenvironment of the nasal epithelium. It was demonstrated that the optimized nasal organoid monolayers adequately recapitulate SARS-CoV-2 high infectivity, particularly the highly transmissible Delta and Omicron variants, in the upper respiratory tract. These optimized nasal organoid monolayers are superior to most, if not all, existing respiratory organoid models. A robust organoid system was generated enabling stable expansion and reconstruction of the human nasal epithelium in culture plates. The nasal organoid culture system provides an unlimited source of physiologically active nasal epithelial cells for studying respiratory pathogens, circumventing the restriction of cultured primary nasal epithelial cells. More importantly, the facile procedure of procuring nasal cells and a zero-failure establishment efficiency allow researchers to establish personalized nasal organoids readily, which will pave a new avenue for many exciting organoid-based investigations for combating the COVID-19 pandemic.

### REFERENCES

1. Chan J F, Yuan S, Kok K H, To K K, Chu H, Yang J, Xing F, Liu J, Yip C C, Poon R W, Tsoi H W, Lo S K, Chan K H, Poon V K, Chan W M, Ip J D, Cai J P, Cheng V C, Chen H, Hui C K, Yuen K Y. 2020. A familial cluster of pneumonia associated with the 2019 novel coronavirus indicating person-to-person transmission: a study of a family cluster. Lancet 395:514-523.
2. Zhu N, Zhang D, Wang W, Li X, Yang B, Song J, Zhao X, Huang B, Shi W, Lu R, Niu P, Zhan F, Ma X, Wang D, Xu W, Wu G, Gao G F, Tan W, China Novel Coronavirus I, Research T. 2020. A Novel Coronavirus from Patients with Pneumonia in China, 2019. N Engl J Med 382:727-733.
3. Gandhi R T, Lynch J B, Del Rio C. 2020. Mild or Moderate Covid-19. N Engl J Med 383:1757-1766.
4. Ziegler C G K, Miao V N, Owings A H, Navia A W, Tang Y, Bromley J D, Lotfy P, Sloan M, Laird H, Williams H B, George M, Drake R S, Christian T, Parker A, Sindel C B, Burger M W, Pride Y, Hasan M, Abraham G E, 3rd, Senitko M, Robinson T O, Shalek A K,

Glover S C, Horwitz B H, Ordovas-Montanes J. 2021. Impaired local intrinsic immunity to SARS-CoV-2 infection in severe COVID-19. Cell 184:4713-4733 e22.
5. Ahn J H, Kim J, Hong S P, Choi S Y, Yang M J, Ju Y S, Kim Y T, Kim H M, Rahman MDT, Chung M K, Hong S D, Bae H, Lee C S, Koh G Y. 2021. Nasal ciliated cells are primary targets for SARS-CoV-2 replication in the early stage of COVID-19. J Clin Invest 131.
6. Deprez M, Zaragosi L E, Truchi M, Becavin C, Ruiz Garcia S, Arguel M J, Plaisant M, Magnone V, Lebrigand K, Abelanet S, Brau F, Paquet A, Pe'er D, Marquette C H, Leroy S, Barbry P. 2020. A Single-Cell Atlas of the Human Healthy Airways. Am J Respir Crit Care Med 202:1636-1645.
7. Vieira Braga F A, Kar G, Berg M, Carpaij O A, Polanski K, Simon L M, Brouwer S, Gomes T, Hesse L, Jiang J, Fasouli E S, Efremova M, Vento-Tormo R, Talavera-Lopez C, Jonker M R, Affleck K, Palit S, Strzelecka P M, Firth H V, Mahbubani K T, Cvejic A, Meyer K B, Saeb-Parsy K, Luinge M, Brandsma C A, Timens W, Angelidis I, Strunz M, Koppelman G H, van Oosterhout A J, Schiller H B, Theis F J, van den Berge M, Nawijn M C, Teichmann S A. 2019. A cellular census of human lungs identifies novel cell states in health and in asthma. Nat Med 25:1153-1163.
8. Campbell F, Archer B, Laurenson-Schafer H, Jinnai Y, Konings F, Batra N, Pavlin B, Vandemaele K, Van Kerkhove M D, Jombart T, Morgan O, le Polain de Waroux O. 2021. Increased transmissibility and global spread of SARS-CoV-2 variants of concern as at June 2021. Euro Surveill 26.
9. Brandal L T, MacDonald E, Veneti L, Ravlo T, Lange H, Naseer U, Feruglio S, Bragstad K, Hungnes O, Odeskaug L E, Hagen F, Hanch-Hansen K E, Lind A, Watle S V, Taxt A M, Johansen M, Vold L, Aavitsland P, Nygard K, Madslien E H. 2021. Outbreak caused by the SARS-CoV-2 Omicron variant in Norway, November to December 2021. Euro Surveill 26.
10. Ravindra N G, Alfajaro M M, Gasque V, Huston N C, Wan H, Szigeti-Buck K, Yasumoto Y, Greaney A M, Habet V, Chow R D, Chen J S, Wei J, Filler R B, Wang B, Wang G, Niklason L E, Montgomery R R, Eisenbarth S C, Chen S, Williams A, Iwasaki A, Horvath T L, Foxman E F, Pierce R W, Pyle A M, van Dijk D, Wilen C B. 2021. Single-cell longitudinal analysis of SARS-CoV-2 infection in human airway epithelium identifies target cells, alterations in gene expression, and cell state changes. PLOS Biol 19: e3001143.
11. Robinot R, Hubert M, de Melo G D, Lazarini F, Bruel T, Smith N, Levallois S, Larrous F, Fernandes J, Gellenoncourt S, Rigaud S, Gorgette O, Thouvenot C, Trebeau C, Mallet A, Dumenil G, Gobaa S, Etournay R, Lledo P M, Lecuit M, Bourhy H, Duffy D, Michel V, Schwartz O, Chakrabarti L A. 2021. SARS-CoV-2 infection induces the dedifferentiation of multiciliated cells and impairs mucociliary clearance. Nat Commun 12:4354.
12. Gamage A M, Tan K S, Chan WOY, Liu J, Tan C W, Ong Y K, Thong M, Andiappan A K, Anderson D E, Wang Y, Wang L F. 2020. Infection of human Nasal Epithelial Cells with SARS-CoV-2 and a 382-nt deletion isolate lacking ORF8 reveals similar viral kinetics and host transcriptional profiles. PLoS Pathog 16: e1009130.
13. Pizzorno A, Padey B, Julien T, Trouillet-Assant S, Traversier A, Errazuriz-Cerda E, Fouret J, Dubois J, Gaymard A, Lescure F X, Duliere V, Brun P, Constant S, Poissy J, Lina B, Yazdanpanah Y, Terrier O, Rosa-Calatrava M. 2020. Characterization and Treatment of SARS-CoV-2 in Nasal and Bronchial Human Airway Epithelia. Cell Rep Med 1:100059.
14. Zhu N, Wang W, Liu Z, Liang C, Wang W, Ye F, Huang B, Zhao L, Wang H, Zhou W, Deng Y, Mao L, Su C, Qiang G, Jiang T, Zhao J, Wu G, Song J, Tan W. 2020. Morphogenesis and cytopathic effect of SARS-CoV-2 infection in human airway epithelial cells. Nat Commun 11:3910.
15. Hou Y J, Okuda K, Edwards C E, Martinez D R, Asakura T, Dinnon K H, 3rd, Kato T, Lee R E, Yount B L, Mascenik™, Chen G, Olivier K N, Ghio A, Tse L V, Leist S R, Gralinski L E, Schafer A, Dang H, Gilmore R, Nakano S, Sun L, Fulcher M L, Livraghi-Butrico A, Nicely N I, Cameron M, Cameron C, Kelvin D J, de Silva A, Margolis D M, Markmann A, Bartelt L, Zumwalt R, Martinez F J, Salvatore S P, Borczuk A, Tata P R, Sontake V, Kimple A, Jaspers I, O'Neal W K, Randell S H, Boucher R C, Baric R S. 2020. SARS-CoV-2 Reverse Genetics Reveals a Variable Infection Gradient in the Respiratory Tract. Cell 182:429-446 e14.
16. Hatton C F, Botting R A, Duenas M E, Haq I J, Verdon B, Thompson B J, Spegarova J S, Gothe F, Stephenson E, Gardner A I, Murphy S, Scott J, Garnett J P, Carrie S, Powell J, Khan C M A, Huang L, Hussain R, Coxhead J, Davey T, Simpson A J, Haniffa M, Hambleton S, Brodlie M, Ward C, Trost M, Reynolds G, Duncan C J A. 2021. Delayed induction of type I and III interferons mediates nasal epithelial cell permissiveness to SARS-CoV-2. Nat Commun 12:7092.
17. Hayflick L, Moorhead P S. 1961. The serial cultivation of human diploid cell strains. Exp Cell Res 25:585-621.
18. Zhou J, Li C, Sachs N, Chiu M C, Wong B H, Chu H, Poon V K, Wang D, Zhao X, Wen L, Song W, Yuan S, Wong K K, Chan J F, To K K, Chen H, Clevers H, Yuen K Y. 2018. Differentiated human airway organoids to assess infectivity of emerging influenza virus. Proc Natl Acad Sci USA 115:6822-6827.
19. Sachs N, Papaspyropoulos A, Zomer-van Ommen D D, Heo I, Bottinger L, Klay D, Weeber F, Huelsz-Prince G, Iakobachvili N, Amatngalim G D, de Ligt J, van Hoeck A, Proost N, Viveen M C, Lyubimova A, Teeven L, Derakhshan S, Korving J, Begthel H, Dekkers J F, Kumawat K, Ramos E, van Oosterhout M F, Offerhaus G J, Wiener D J, Olimpio E P, Dijkstra K K, Smit E F, van der Linden M, Jaksani S, van de Ven M, Jonkers J, Rios A C, Voest E E, van Moorsel C H, van der Ent C K, Cuppen E, van Oudenaarden A, Coenjaerts F E, Meyaard L, Bont L J, Peters P J, Tans S J, van Zon J S, Boj S F, Vries R G, Beekman J M, Clevers H. 2019. Long-term expanding human airway organoids for disease modeling. EMBO J 38.
20. Mallapaty S. 2021. The mini lungs and other organoids helping to beat COVID. Nature 593:492-494.
21. Li C, Chiu M C, Yu Y, Liu X, Xiao D, Huang J, Wan Z, Zhou J. 2022. Establishing Human Lung Organoids and Proximal Differentiation to Generate Mature Airway Organoids. JoVE doi:doi:10.3791/63684: e63684.
22. Chiu M C, Li C, Liu X, Yu Y, Huang J, Wan Z, Xiao D, Chu H, Cai J P, Zhou B, Sit K Y, Au W K, Wong K K, Li G, Chan J F, To K K, Chen Z, Jiang S, Clevers H, Yuen K Y, Zhou J. 2022. A bipotential organoid model of respiratory epithelium recapitulates high infectivity of SARS-CoV-2 Omicron variant. Cell Discov 8:57.
23. Lamers M M, Beumer J, van der Vaart J, Knoops K, Puschhof J, Breugem T I, Ravelli R B G, Paul van Schayck J, Mykytyn A Z, Duimel H Q, van Donselaar E, Riesebosch S, Kuijpers H J H, Schipper D, van de Wetering W J, de Graaf M, Koopmans M, Cuppen E, Peters P J, Haagmans B L, Clevers H. 2020. SARS-CoV-2 productively infects human gut enterocytes. Science 369:50-54.
24. Mykytyn A Z, Breugem T I, Riesebosch S, Schipper D, van den Doel P B, Rottier R J, Lamers M M, Haagmans B L. 2021. SARS-CoV-2 entry into human airway organoids is serine protease-mediated and facilitated by the multibasic cleavage site. Elife 10.
25. Chaturvedi S, Vasen G, Pablo M, Chen X, Beutler N, Kumar A, Tanner E, Illouz S, Rahgoshay D, Burnett J, Holguin L, Chen P Y, Ndjamen B, Ott M, Rodick R, Rogers T, Smith D M, Weinberger L S. 2021. Identification of a therapeutic interfering particle-A single-dose SARS-CoV-2 antiviral intervention with a high barrier to resistance. Cell 184:6022-6036 e18.
26. Wang D, Li C, Chiu M C, Yu Y, Liu X, Zhao X, Huang J, Cheng Z, Yuan S, Poon V, Cai J P, Chu H, Chan J F, To K K, Yuen K Y, Zhou J. 2022. SPINK6 inhibits human airway serine proteases and restricts influenza virus activation. EMBO Mol Med 14: e14485.
27. Sato T, Stange D E, Ferrante M, Vries R G, Van Es J H, Van den Brink S, Van Houdt W J, Pronk A, Van Gorp J, Siersema P D, Clevers H. 2011. Long-term expansion of epithelial organoids from human colon, adenoma, adenocarcinoma, and Barrett's epithelium. Gastroenterology 141:1762-72.
28. Sato T, Vries R G, Snippert H J, van de Wetering M, Barker N, Stange D E, van Es J H, Abo A, Kujala P, Peters P J, Clevers H. 2009. Single Lgr5 stem cells build crypt-villus structures in vitro without a mesenchymal niche. Nature 459:262-5.
29. Kayisoglu O, Weiss F, Niklas C, Pierotti I, Pompaiah M, Wallaschek N, Germer C T, Wiegering A, Bartfeld S. 2021. Location-specific cell identity rather than exposure to G I microbiota defines many innate immune signalling cascades in the gut epithelium. Gut 70:687-697.
30. Pan Y, Zhang D, Yang P, Poon LLM, Wang Q. 2020. Viral load of SARS-CoV-2 in clinical samples. Lancet Infect Dis 20:411-412.
31. Co J Y, Margalef-Catala M, Li X, Mah A T, Kuo C J, Monack D M, Amieva M R. 2019. Controlling Epithelial Polarity: A Human Enteroid Model for Host-Pathogen Interactions. Cell Rep 26:2509-2520 e4.
32. Fischer H, Widdicombe J H. 2006. Mechanisms of acid and base secretion by the airway epithelium. J Membr Biol 211:139-50.
33. Sharma N K, Sarode S C. 2021. Low pH and temperature of airway surface liquid are key determinants that potentiate SARS-CoV-2 infectivity. Curr Mol Med doi:10.2174/1566524021666210816095557.
34. Jimenez L, Campos Codo A, Sampaio V S, Oliveira A E R, Ferreira L K K, Davanzo G G, de Brito Monteiro L, Victor Virgilio-da-Silva J, Borba M G S, Fabiano de Souza G, Zini N, de Andrade Gandolfi F, Muraro S P, Luiz Proenca-Modena J, Val F A, Cardoso Melo G, Monteiro W M, Nogueira M L, Lacerda M V G, Moraes-Vieira P M, Nakaya H I. 2021. Acid pH Increases SARS-CoV-2 Infection and the Risk of Death by COVID-19. Front Med (Lausanne) 8:637885.
35. Linfield D T, Raduka A, Aghapour M, Rezaee F. 2021. Airway tight junctions as targets of viral infections. Tissue Barriers 9:1883965.
36. Sungnak W, Huang N, Beavin C, Berg M, Queen R, Litvinukova M, Talavera-López C, Maatz H, Reichart D, Sampaziotis F, Worlock K B, Yoshida M, Barnes J L, Banovich N E, Barbry P, Brazma A, Collin J, Desai T J, Duong T E, Eickelberg O, Falk C, Farzan M, Glass I, Gupta R K, Haniffa M, Horvath P, Hubner N, Hung D, Kaminski N, Krasnow M, Kropski J A, Kuhnemund M, Lako M, Lee H, Leroy S, Linnarson S, Lundeberg J, Meyer K B, Miao Z, Misharin A V, Nawijn M C, Nikolic M Z, Noseda M, Ordovas-Montanes J, Oudit G Y, Pe'er D, Powell J, Quake S, Rajagopal J, Tata P R, et al. 2020. SARS-CoV-2 entry factors are highly expressed in nasal epithelial cells together with innate immune genes. Nature Medicine 26:681-687.
37. Zachos N C, Kovbasnjuk O, Foulke-Abel J, In J, Blutt S E, de Jonge H R, Estes M K, Donowitz M. 2016. Human Enteroids/Colonoids and Intestinal Organoids Functionally Recapitulate Normal Intestinal Physiology and Pathophysiology. J Biol Chem 291:3759-66.
38. Hogan B L, Barkauskas C E, Chapman H A, Epstein J A, Jain R, Hsia C C, Niklason L, Calle E, Le A, Randell S H, Rock J, Snitow M, Krummel M, Stripp B R, Vu T, White E S, Whitsett J A, Morrisey E E. 2014. Repair and regeneration of the respiratory system: complexity, plasticity, and mechanisms of lung stem cell function. Cell Stem Cell 15:123-38.
39. Pardo-Saganta A, Law B M, Tata P R, Villoria J, Saez B, Mou H, Zhao R, Rajagopal J. 2015. Injury induces direct lineage segregation of functionally distinct airway basal stem/progenitor cell subpopulations. Cell Stem Cell 16:184-97.
40. Salahudeen A A, Choi S S, Rustagi A, Zhu J, van Unen V, de la O S, Flynn R A, Margalef-Catala M, Santos A J M, Ju J, Batish A, Usui T, Zheng G X Y, Edwards C E, Wagar L E, Luca V, Anchang B, Nagendran M, Nguyen K, Hart D J, Terry J M, Belgrader P, Ziraldo S B, Mikkelsen T S, Harbury P B, Glenn J S, Garcia K C, Davis M M, Baric R S, Sabatti C, Amieva M R, Blish C A, Desai T J, Kuo C J. 2020. Progenitor identification and SARS-CoV-2 infection in human distal lung organoids. Nature 588:670-675.
41. Lee J H, Tammela T, Hofree M, Choi J, Marjanovic N D, Han S, Canner D, Wu K, Paschini M, Bhang D H, Jacks T, Regev A, Kim C F. 2017. Anatomically and Functionally Distinct Lung Mesenchymal Populations Marked by Lgr5 and Lgr6. Cell 170:1149-1163 e12.
42. Tadokoro T, Gao X, Hong C C, Hotten D, Hogan B L. 2016. BMP signaling and cellular dynamics during regeneration of airway epithelium from basal progenitors. Development 143:764-73.
43. Zhao H, Lu L, Peng Z, Chen L L, Meng X, Zhang C, Ip J D, Chan W M, Chu A W, Chan K H, Jin D Y, Chen H, Yuen K Y, To K K. 2022. SARS-CoV-2 Omicron variant shows less efficient replication and fusion activity when compared with Delta variant in TMPRSS2-expressed cells. Emerg Microbes Infect 11:277-283.
44. Rajan A, Weaver A M, Aloisio G M, Jelinski J, Johnson H L, Venable S F, McBride T, Aideyan L, Piedra F A, Ye X, Melicoff-Portillo E, Yerramilli MRK, Zeng X L, Mancini M A, Stossi F, Maresso A W, Kotkar S A, Estes M K, Blutt S, Avadhanula V, Piedra P A. 2022. The Human Nose Organoid Respiratory Virus Model: an Ex Vivo Human Challenge Model To Study Respiratory Syncytial Virus (RSV) and Severe Acute Respiratory Syndrome Coronavirus 2 (SARS-CoV-2) Pathogenesis and Evaluate Therapeutics. mBio doi:10.1128/mbio.03511-21: e0351121.
45. Zhou J, Wang D, Wong B H, Li C, Poon V K, Wen L, Zhao X, Chiu M C, Liu X, Ye 45. Z, Yuan S, Sze K H, Chan J F, Chu H, To K K, Yuen K Y. 2019. Identification and characterization of GLDC as host susceptibility gene to severe influenza. EMBO Mol Med 11.
46. Zhao X, Li C, Liu X, Chiu M C, Wang D, Wei Y, Chu H, Cai J P, Hau-Yee Chan I, Kak-Yuen Wong K, Fuk-Woo Chan J, Kai-Wang To K, Yuen K Y, Zhou J. 2021. Human Intestinal Organoids Recapitulate Enteric Infections of Enterovirus and Coronavirus. Stem Cell Reports 16:493-504.
47. Zhou J, Li C, Liu X, Chiu M C, Zhao X, Wang D, Wei Y, Lee A, Zhang A J, Chu H, Cai J P, Yip C C, Chan I H, Wong K K, Tsang O T, Chan K H, Chan J F, To K K, Chen H, Yuen K Y. 2020. Infection of bat and human intestinal organoids by SARS-CoV-2. Nat Med 26:1077-1083.
48. Peacock T P, Brown J C, Zhou J, Thakur N, Newman J, Kugathasan R, Sukhova K, Kaforou M, Bailey D, Barclay W S. 2022. The SARS-CoV-2 variant, Omicron, shows rapid replication in human primary nasal epithelial cultures and efficiently uses the endosomal route of entry. bioRxiv doi:10.1101/2021.12.31.474653:2021.12.31.474653.
49. Lamers M M, Mykytyn A Z, Breugem T I, Groen N, Knoops K, Schipper D, van Acker R, van den Doel P B, Bestebroer T, Koopman C D, Reusken C, Muraro M J, GeurtsvanKessel C H, van Royen M E, Peters P J, Zhang J, Haagmans B L. 2022. SARS-CoV-2 Omicron efficiently infects human airway, but not alveolar epithelium. bioRxiv doi:10.1101/2022.01.19.476898:2022.01.19.476898.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Those skilled in the art will recognize, or be able to ascertain using no more than routine experimentation, many equivalents to the specific embodiments of the method and compositions described herein. Such equivalents are intended to be encompassed by the following claims.

1. An in vitro generated organoid comprising a population of differentiated nasal epithelial cells having cellular heterogeneity and molecular markers similar to mature nasal epithelial tissue.

2. (canceled)

3. The organoid of claim 1, wherein the population of differentiated nasal epithelial cells expresses one or more molecular markers selected from the group comprising Angiotensin-converting enzyme-2 (ACE2), Transmembrane serine protease 2 (TMPRSS2), forkhead-box G1 (FOXG1), SIX3, paired box-7 (PAX7), P63, Keratin 5 (CK5), Sentan (SNTN), Mucin 5AC (MUC5AC), and CC10; and
wherein the population of differentiated nasal epithelial cells express lower levels of trachea/bronchi-associated genes selected from the group comprising FOLR1, LMO3, and IRX2.

4. (canceled)

5. The organoid of claim 1, wherein the cellular heterogeneity comprises having a mixture of basal cells, ciliated cells, goblet cells, and club cells.

6. The organoid of claim 1, wherein the population of differentiated nasal epithelial cells is organized in a three-dimensional structure, and is encapsulated within a hydrogel or embedded within a biocompatible scaffold that provides structural support and promotes tissue organization.

7. The organoid of claim 6, wherein the population of differentiated nasal epithelial cells comprises basal cells at a percentage of about 12% to about 35%, ciliated cells at a percentage of about 35% to about 45%, goblet cells at a percentage of about 5% to about 10%, and club cells at a percent of about 0.1% to about 5%; and
ACE2 is expressed by about 20% to about 45% of the population of differentiated nasal epithelial cells, and TMPRSS2 is expressed by about 40% to about 55% of the population of differentiated nasal epithelial cells.

8. (canceled)

9. The organoid culture system of claim 1, wherein the population of differentiated nasal epithelial cells are organized in a two-dimensional (2D) monolayer.

10. The organoid culture system of claim 6, wherein the population of differentiated nasal epithelial cells are incubated for a period of time during differentiation in a medium having a slightly acidic pH of about 6.0 to about 6.9, preferably about 6.6.

11. The organoid culture system of claim 9, wherein the population of differentiated nasal epithelial cells comprise basal cells at a percentage of about 1% to about 10%, ciliated cells at a percentage of about 75% to about 92%, goblet cells at a percentage of about 4% to about 22%, and club cells at a percent of about 0.05% to about 2%; and
wherein ACE2 is expressed by about 30% to about 50% of the population of differentiated nasal epithelial cells, and TMPRSS2 is expressed by about 85% to about 100% of the population of differentiated nasal epithelial cells.

12. (canceled)

13. The organoid of claim 1, wherein the population of differentiated nasal epithelial cells are derived from undifferentiated nasal cells obtained non-invasively from a subject, wherein the subject is preferably a human.

14. The organoid of claim 1, wherein the population of differentiated nasal epithelial cells are genetically modified to increase or reduce expression of specific genes involved in nasal tissue development, function, or disease, and
wherein the population of differentiated nasal epithelial cells exhibit functional properties, including mucus production, ciliary beating, and responses to olfactory stimuli.

15. (canceled)

16. A method for generating a three-dimensional (3D) organoid of the nasal epithelium comprising the steps of:
(i) adhering cells onto a support structure that promotes the formation of (3D) organoids, wherein the cells are nasal turbinate cells capable of differentiating into nasal epithelial cells;
(ii) culturing the nasal turbinate cells on the support structure in an expansion culture medium for a period of time to produce 3D human nasal undifferentiated cells, wherein the period of time is about 8 days to about 12 days, preferably about 10 days;
(iii) culturing the 3D nasal undifferentiated cells on the support structure in a basal medium for period of time to enlarge the central lumen, wherein the period of time is about 3 days to about 5 days, preferably about 4 days;
(iv) culturing the 3D nasal undifferentiated cells on the support structure produced in step (iii) in a proximal differentiation medium for a period of time to generate the 3D differentiated organoid of the human nasal epithelium, wherein the period of time is about 5 days to about 7 days, preferably about 6 days.

17. The method of claim 16, wherein the expansion culture medium comprises basal media comprising one or more buffering agents, one or more growth factors, a ROCK inhibitor, a TGF β inhibitor, a MAPK inhibitor, a WNT agonist, a BMP-4 inhibitor, one or more supplements, and one or more antibiotics;
wherein the basal media is Dulbecco's Modified Eagle's Medium (DMEM), Advanced DMEM/F12, RPMI-1640, or Minimum Essential Medium (MEM);
wherein the buffering agent is HEPES buffer present in an amount ranging from 0.5% to about 1.5%, preferably about 1.0%;
wherein the one or more growth factors are selected from the group comprising Epidermal growth factor (EGF) and fibroblast growth factor (FGF), wherein the growth factors are preferably FGF-7 in a concentration from about 3 ng/ml to about 7 ng/ml, preferably about 5 ng/ml, and FGF-10 in a concentration from about 15 ng/ml to about 25 ng/ml, preferably about 20 ng/ml;
wherein the ROCK inhibitor is Y-27632 in a concentration from about 3 μM to about 7 μM, preferably about 5 μM;
wherein the TGF-β inhibitor is A8301 in a concentration from about 400 nM to about 600 nM, preferably about 500 nM;
wherein the MAPK inhibitor is SB202190 in a concentration from about 0.5 μM to about 1.5 μM, preferably 1.0 μM;
wherein the WNT agonist is R Spondin 1 present in an amount from about 8% to about 12%, preferably about 10%;
wherein the BMP-4 inhibitor is Noggin present in a concentration of about 8% to about 12%, preferably about 10%;
wherein the one or more supplements are selected from the group comprising GlutaMAX® in an amount from about 0.5% to about 1.5%, preferably about 1%, B27 supplement, N-acetylcysteine in concentration from about 1.0 mM to about 1.5 mM, preferably about 1.25 mM, and nicotinamide in concentration from about 7 mM to about 13 mM, preferably about 10 mM;
wherein the one or more antibiotics are selected from the group comprising Penicillin-Streptomycin in an amount from about 0.5% to about 1.5%, preferably about 1.0%, and Primocin in a concentration from about 75 μg/ml to about 125 μg/ml, preferably about 100 μg/ml;
wherein the basal medium is Advanced DMEM/F-12 supplemented with about 1% HEPES buffer, about 1% GlutaMAX®, and about 1% Penicillin-Streptomycin;
wherein the proximal differentiation medium comprises basal medium supplemented with a γ-secretase inhibitor in a concentration from about 7 UM to about 13 M, preferably about 10 μM, and
wherein the proximal differentiation medium has a pH of about 7.2 to about 7.6, preferably about 7.4.

18. The method of claim 16, wherein the support structure comprises materials selected from the group comprising Matrigel®, collagen, hyaluronic acid extracellular matrix, synthetic hydrogels, decellularized extracellular matrix, alginate, Poly (lactic-co-glycolic acid) (PLGA), or electrospun fibers, preferably Matrigel®.

19. (canceled)

20. (canceled)

21. The method of claim 16, further comprising: (v) replenishing the expansion medium once a day, three times a week, or 4 times a week, and (vi) re-plating and passaging 3D human nasal undifferentiated cells one or more times.

22. (canceled)

23. A method for deriving a two-dimensional (2D) organoid monolayer of the nasal epithelium from the 3D nasal undifferentiated cells generated by step (ii) of claim 16, the method comprising:
(a) dissociating the 3D nasal undifferentiated cells into a plurality of single nasal undifferentiated cells;
(b) transferring the plurality of single nasal undifferentiated cells to a two-chamber support structure and culturing the plurality of single nasal undifferentiated cells in expansion medium for a period of time to allow cell attachment and expansion, wherein the period of time is about 1 day to about 3 days; and
(c) culturing the expanded nasal undifferentiated cells in a first and second proximal differentiation medium for a total of about 8 days to generate confluent two-dimensional (2D) organoid monolayers.

24. The method of claim 23, wherein the two-chamber support structure comprises a top-chamber and bottom-chamber, and wherein the single nasal undifferentiated cells are adhered to the top chamber of the support structure.

25. The method of claim 23, wherein the culturing of step (c) comprises:
adding the first proximal differentiation medium to the top chamber and the bottom chamber, wherein the first proximal differentiation medium has a pH of about 7.2 to about 7.6, preferably about 7.4;
incubating the nasal undifferentiated cells for about 2 days;
replacing the first proximal differentiation medium in the top chamber with the second proximal differentiation medium, wherein the second proximal differentiation medium has a pH of about 6.4 to about 6.8, preferably about 6.6; and
incubating the nasal undifferentiated cells for about 6 days.

26. A method for screening an agent for an effect on the nasal epithelium, the method comprising contacting the in vitro generated organoid of claim 1 with the agent, and determining the effect of the agent on survival, proliferation, differentiation, morphologic parameters, genetic parameters, or functional parameters of the in vitro generated organoid.

27. The method of claim 26, wherein the agent is an antiviral agent, an antibacterial agent, an antifungal agent, or an anti-allergenic agent, wherein the effect of the agent is indicative of the agent being safe for the treatment of respiratory infections; or
wherein the agent is nucleic acids or analogs thereof, polypeptides or analogs thereof, antibodies, chemicals, small molecules, and/or any combination thereof.

28. (canceled)

29. A kit for generating a two-dimensional monolayer or three-dimensional organoid of the human nasal epithelium, the kit comprising a combination of two or more of:
(i) Expansion culture medium for deriving the 3D undifferentiated nasal cells comprising basal media comprising one or more of a buffering agent, a growth factor, a ROCK inhibitor, a TGF β inhibitor, a MAPK inhibitor, a WNT agonist, a BMP-4 inhibitor, one or more supplements, and one or more antibiotics;
(ii) Basal media comprising Advanced DMEM/F-12 supplemented with a HEPES buffer, GlutaMAX®, and an antibiotic;
(iii) one or more proximal differentiation media comprising basal medium supplemented with a γ-secretase inhibitor; and
(iv) one or more scaffold or support structures.
